(19) United States
(12) Patent Application Publication
(10) Pub. No.: US 2011/0045007 A1
(43) Pub. Date: Feb. 24, 2011

(54) FUSION OR LINKED PROTEINS WITH EXTENDED HALF LIFE

(75) Inventors: Janine Schuurman, Diemen (NL); Tom Vink, Alphen aan den Rijn (NL); Jan Van De Winkel, Zeist (NL); Aran Frank Labrljn, Nigtevecht (NL); Paul Parren, odijk (NL); Willem Karel Bleeker, Amsterdam (NL); FRank Beurskens, Culemborg (NL); Patrick Van Berkel, Utrecht (NL)

Correspondence Address: NELSON MULLINS RILEY & SCARBOROUGH LLP;FLOOR 30, SUITE 3000, ONE POST OFFICE SQUARE, BOSTON, MA, 02109, US

(73) Assignee: GENMAB A/S, Copenhagen (DK)

(21) Appl. No.: 12/602,416

(22) PCT Filed: May 30, 2008
(86) PCT No.: PCT/DK08/50126

§ 371 Date: Jul. 6, 2010

(30) Foreign Application Priority Data
May 31, 2007 (DK) PA 2007 00792

(51) Int. Cl. A61K 39/395; C12P 21/02; C12N 9/96; C12N 5/071; C12N 5/10; C12N 1/21; C12N 1/15; C12N 15/63; C07K 16/00; C07K 19/00; C07K 16/46; A61P 29/00; A61P 35/00; A61P 3/10; A61P 25/16; A61P 25/08; A61P 25/18; A61P 25/00; A61P 31/04; A61P 31/12; A61P 31/10; A61P 7/00; A61P 11/06; A61P 25/24; A61P 7/02; A61P 33/00

(52) U.S. Cl. 424/179.1; 424/178.1; 435/69.6; 435/188; 435/325; 435/419; 435/252.33; 435/254.11; 435/320.1; 530/303; 530/351; 530/387.1; 530/391.1; 530/391.7; 530/391.9

(57) ABSTRACT

The present invention provides fusion proteins comprising a first molecule, and a second molecule which is a monovalent immunoglobulin or a fragment of a monovalent immunoglobulin with a long half-life when administered in vivo, methods of making such fusion proteins, pharmaceutical compositions comprising such fusion proteins, and uses thereof.

### FIELD OF THE INVENTION

The present invention relates to fusion or linked proteins wherein a monovalent immunoglobulin or a fragment thereof, comprising at least the CH2 and CH3 regions are fused or linked to an other protein or pharmaceutical entity, to provide a molecule with an extended in vivo half-life.

### BACKGROUND OF THE INVENTION

Therapeutic proteins (e.g. cytokines, soluble cytokine receptors, etc) have revolutionized the treatment of many diseases, but low activity or rapid clearance limits their utility. New approaches have been taken to design drugs with enhanced in vivo activity and/or half-life to reduce injection frequency, increase convenience and improve patient compliance. Strategies to prolong the serum half-life of therapeutic proteins include PEGylation, glycoengineering and fusing to protein domains with long-serum half-lives. A frequently used protein domain for this purpose is the Fc-domain of the immunoglobulin molecule. The mechanism by which the Fc-domain increases half-life is two-fold. First, by addition of an Fc-domain the molecular size of the protein increases (+50 kD), thus making it too big for renal exclusion, and secondly by transferring the protective properties of the Fc-domain on immunoglobulin catabolism to the protein, mediated through neonatal Fc receptor (FcRn) binding. A potential draw-back of the Fc-domain, however, is that it naturally forms homodimers, making the therapeutic protein functionally bivalent. Furthermore, the Fc-domain of an IgG1 can mediate effector functions (CDC, ADCC), which could lead to unwanted inflammation. In the present invention, we use our discovery that monovalent Fc-domains lack effector function, and therefore may be used as a fusion partner for various peptides for therapeutic use, in cases where bivalency and effector functions are unwanted. Furthermore, we have discovered that the half-life of the monovalent immunoglobulin is independent of glycosylation status of the molecule, indicating that the molecules could be produced in expression systems that do not confer glycosylation onto the expressed protein, such as in bacteria, thereby allowing cheap production of the proteins.

Another approach to effectively prolong serum half-life of therapeutic proteins is by transferring the protective FcRn interaction using a therapeutic protein-specific antibody. This approach would not only be applicable to administered (exogenous) proteins, but also to endogenous proteins. Experimental evidence supporting this approach comes from several anti-cytokine antibodies that were tested in animal models. Administration of a neutralizing IL-6 antibody alone resulted in sustained elevation of circulating (endogenous) IL-6 in mice and baboons (May, L T, et al; J Immunol 1993). Furthermore, injection of cytokine-anti-cytokine (neutralizing) antibody complexes (IL-3, IL-4 and IL-7) in mice resulted in prolongation of in vivo effects of exogenous cytokines (Finkelman, F D, et al; J Immunol 1993). These data indeed suggest that antibodies can act as chaperones and increase serum half-lives of their cytokine target. Studies with an anti-MCP-1 antibody (Haringman, J J, et al; Arthritis Rheum 2006) and anti-Botulinum Neurotoxin antibodies (Marks, J D; Abstract 027; Antibodies as drugs, Keystone Symposium 2007; Lake Louise), reporting similar effects, suggest that this is not restricted to cytokines, but is a more general phenomenon. Our discoveries that monovalent immunoglobulins of the invention have a long halflife, and do not exhibit any effector functions, indicate that such protein specific monovalent immunoglobulins advantageously may be used to target endogenous and exogenous cytokines and other peptides for the purpose of extending their in vivo half-life, in cases where effector function of the immunoglobulin is unwanted.

### SUMMARY OF THE INVENTION

The present invention relates to a novel class of fused or linked proteins with a long in vivo half life, comprising a first molecule which is fused to a monovalent immunoglobulin or a fragment of a monovalent immunoglobulin. The presence of the monovalent immunoglobulin provide an extended half life to the other part of the fusion molecule, which may be a therapeutic molecule. Furthermore, the monovalent immunoglobulin or fragment thereof is unable to induce effector functions such as ADCC, which in some applications is an advantage over in example dimeric immunoglobulin fragments comprising the CH2 and CH3 regions. The fusion proteins of the present invention are useful for therapeutic applications, wherein an extended in vivo half life of the therapeutic molecule is favorable, and wherein ADCC is undesirable.

The present invention provides:
1) A fusion protein comprising at least first molecule and a monovalent immunoglobulin or a fragment of a monovalent immunoglobulin, wherein
   i) said first molecule and said monovalent immunoglobulin or fragment of a monovalent immunoglobulin are fused or linked either by peptide bonds or by other types of covalent bonding, and
   ii) the monovalent immunoglobulin or the fragment of a monovalent immunoglobulin comprises at least the CH2 and CH3 regions of the CH, and
   iii) wherein the monovalent immunoglobulin or fragment thereof as required by the Ig subtype, has been modified such that the CH3 region or other regions do not comprise any amino acid residues which are capable of participating in the formation of disulphide bonds or covalent or stable non-covalent inter-heavy chain bonds with other peptides comprising an identical amino acid sequence of the CH region of the immunoglobulin in the presence of polyclonal human Ig;
   iv) if a C_{L} region or a fragment of the C_{L} region is not present, said monovalent immunoglobulin or fragment of a monovalent immunoglobulin does not comprise the CH1 region or has a CH1 region, where one or more amino acids in the hydrophobic patch have been changed to amino acids that are less hydrophobic, unless the CH1 region is of the camelidae type, and
   v) the hinge region of said monovalent immunoglobulin or fragment of the monovalent immunoglobulin is either deleted or mutated to remove any cysteine residues.
2) A fusion protein according to 1, wherein the first molecule is one of the following: a cytokine, a polypeptide, a peptide mimetic, a small organic molecule.
3) A fusion protein according to 1 and 2, wherein the monovalent immunoglobulin or fragment thereof does not comprise any variable regions derived from an immunoglobulin.
4) A fusion protein according to any one of 1-3, wherein the hinge region of the monovalent immunoglobulin or fragment thereof has been deleted.
5) A fusion protein according to any one of 1 to 4, wherein the cytokine or other peptide is fused to the N-terminal of the monovalent immunoglobulin or fragment thereof.
6) A fusion protein according to any one of 1-2 and 4-5, wherein a C_{L} region or a fragment of a C_{L} region is present.
7) A fusion protein according to any one of the 1 to 4, wherein the monovalent immunoglobulin or fragment of a monovalent immunoglobulin consist of
   i) The CH1 and CH2 domains of the CH, or
   ii) The CH domain, or
   iii) The VH and CH domains, or
   iv) The VH, CH, and CL domains, or
   v) The VH, CH, VL and CL domains
8) A fusion protein according to 6, which comprises two polypeptides fused to the monovalent immunoglobulin, wherein a first polypeptide is fused or linked to the heavy chain of the monovalent immunoglobulin and one is fused or linked to the light chain.
9) A fusion protein according to 6, wherein the two polypeptides fused to the monovalent immunoglobulin are different.
10) A fusion protein according to any one of 1-3, wherein
   i) the monovalent antibody or fragment thereof comprises an antigen binding region, and
   ii) a linker molecule comprising one part that is capable of being bound by said antigen binding region, and one part that is capable of binding a cytokine or other peptide, and
   iii) a cytokine or other peptide which is capable of being bound by the linker of ii)
11) A fusion protein according to any one of 1 to 9, wherein the cytokine or other peptide is fused to the monovalent immunoglobulin or fragment thereof by peptide bonding, and wherein one or more amino acids have been inserted as spacers between the two peptides.
12) A fusion protein according to any one of 1 to 9, wherein the cytokine or other peptide is covalently linked to the monovalent immunoglobulin or fragment thereof by other covalent bonds than peptide bonding, and wherein a linker molecule is inserted between the two peptides.
13) A fusion protein according to any one of 1-12, wherein the Ig is IgG1, IgG2, IgG3, IgG4, IgA1, IgA2 or IgD.
14) A fusion protein according to any one of 1-13, wherein the Ig is human.
15) A fusion protein according to 14, wherein the Ig is a fragment of human IgG1 having the amino acid C_{H}3 region as set forth in SEQ ID NO: 19, wherein the C_{H}3 region has been modified so that one or more of the following amino acid substitutions have been made: Arg (R) in position 238 has been replaced by Gln (Q); Asp (D) in position 239 has been replaced by Glu (E); Thr (T) in position 249 has been replaced by Ala (A); Leu (L) in position 251 has been replaced by Ala (A); Leu (L) in position 251 has been replaced by Val (V); Phe (F) in position 288 has been replaced by Ala (A); Phe (F) in position 288 has been replaced by Leu (L); Tyr (Y) in position 290 has been replaced by Ala (A); Lys (K) in position 292 has been replaced by Arg (R); Lys (K) in position 292 has been replaced by Ala (A); Gln (Q) in position 302 has been replaced by Glu (E); and Pro (P) in position 328 has been replaced by Leu (L).
16) A fusion protein according to 15, wherein Arg (R) in position 238 has been replaced by Gln (Q).
17) A fusion protein according to 15, wherein Arg (R) in position 238 has been replaced by Gln (Q), and Pro (P) in position 328 has been replaced by Leu (L).
18) A fusion protein according to 15, wherein all nine amino acids have been substituted.
19) A fusion protein according to 14, wherein the human Ig is a fragment of IgG2 having the amino acid C_{H}3 region as set forth in SEQ ID NO: 20, wherein the C_{H}3 region has been modified so that one or more of the of the following amino acid substitutions have been made: Arg (R) in position 234 has been replaced by Gln (Q); Thr (T) in position 245 has been replaced by Ala (A); Leu (L) in position 247 has been replaced by Ala (A); Leu (L) in position 247 has been replaced by Val (V); Met (M) in position 276 has been replaced by Val (V); Phe (F) in position 284 has been replaced by Ala (A); Phe (F) in position 284 has been replaced by Leu (L); Tyr (Y) in position 286 has been replaced by Ala (A); Lys (K) in position 288 has been replaced by Arg (R); Lys (K) in position 288 has been replaced by Ala (A); Gln (Q) in position 298 has been replaced by Glu (E); and Pro (P) in position 324 has been replaced by Leu (L).
20) A fusion protein according to 19, wherein Arg (R) in position 234 has been replaced by Gln (Q).
21) A fusion protein according to 19, wherein Arg (R) in position 234 has been replaced by Gln (Q); and Pro (P) in position 324 has been replaced by Leu (L).
22) A fusion protein according to 19, wherein all nine amino acids have been substituted.
23) A fusion protein according to 14, wherein the Ig is a fragment of an human IgG3 having the amino acid C_{H} region as set forth in SEQ ID NO: 21, wherein the C_{H}3 region has been modified so that one or more of the following amino acid substitutions have been made: Arg (R) in position 285 has been replaced by Gln (Q); Thr (T) in position 296 has been replaced by Ala (A); Leu (L) in position 298 has been replaced by Ala (A); Leu (L) in position 298 has been replaced by Val (V); Ser (S) in position 314 has been replaced by Asn (N); Asn (N) in position 322 has been replaced by Lys (K); Met (M) in position 327 has been replaced by Val (V); Phe (F) in position 335 has been replaced by Ala (A); Phe (F) in position 335 has been replaced by Leu (L); Tyr (Y) in position 337 has been replaced by Ala (A); Lys (K) in position 339 has been replaced by Arg (R); Lys (K) in position 339 has been replaced by Ala (A); Gln (Q) in position 349 has been replaced by Glu (E); Ile (I) in position 352 has been replaced by Val (V); Arg (R) in position 365 has been replaced by His (H); Phe (F) in position 366 has been replaced by Tyr (Y); and Pro (P) in position 375 has been replaced by Leu (L).
24) A fusion protein according to 23, wherein Arg (R) in position 285 has been replaced by Gln (Q).
25) A fusion protein according to 23, wherein Arg (R) in position 285 has been replaced by Gln (Q); and Pro (P) in position 375 has been replaced by Leu (L).
26) A fusion protein according to 23, wherein all 14 amino acids have been substituted.
27) A fusion protein according to any one of 1-14, wherein the monovalent immunoglobulin or fragment thereof comprises the human IgG4 CH2 and CH3 sequence of SEQ ID NO: 16.
28) A fusion protein according to any one of 1-14, wherein the monovalent immunoglobulin or fragment thereof consists of the human IgG4 CH2 and CH3 sequence of SEQ ID NO: 16.
29) A fusion protein according to any one of 1-14, wherein the Ig is a fragment of the human IgG4 CH2 and CH3 sequence as set forth in SEQ ID NO: 16, and wherein the C_{H}3 region has been modified so that one or more of the following amino acid substitutions have been made: Thr (T) in position 234 has been replaced by Ala (A); Leu (L) in position 236 has been replaced by Ala (A); Leu (L) in position 236 has been replaced by Val (V); Phe (F) in position 273 has been replaced by Ala (A); Phe (F) in position 273 has been replaced by Leu (L); Tyr (Y) in position 275 has been replaced by Ala (A); Arg (R) in position 277 has been replaced by Ala (A);
30) A fusion protein according to 29, wherein all 4 amino acids have been substituted.
31) A fusion protein according to any of 1-30, wherein the amino acid sequence corresponding to the hinge region of the heavy chain fragment of said immunoglobulin has been deleted.
32) A fusion protein according to any one of 1-14 and 27-30, wherein the amino acid sequence of a heavy chain of a human IgG4, has been modified such that said heavy chain fragment comprises a C_{H} region, wherein the amino acid residues corresponding to amino acid residues 106 and 109 of the sequence of SEQ ID No: 14 have been substituted with amino acid residues different from cysteine.
33) A fusion protein according to any one of the preceding 1-30, wherein the C_{H} region of the monovalent antibody or fragment thereof, has been modified such that the region corresponding to the hinge region of the C_{H} region does not comprise any cysteine residues.
34) A fusion protein according to any one of the preceding 1-30, wherein the C_{H} region has been modified such that at least all cysteine residues have been deleted and/or substituted with other amino acid residues.
35) A fusion protein according to 34, wherein the C_{H} region has been modified such that the cysteine residues of the hinge region have been substituted with amino acid residues that have an uncharged polar side chain, or a nonpolar side chain.
36) A fusion protein according to 35, wherein the amino acids with uncharged polar side chains are independently selected from asparagine, glutamine, serine, threonine, tyrosine, and tryptophan, and the amino acid with the nonpolar side chain are independently selected from alanine, valine, leucine, isoleucine, proline, phenylalanine, and methionine.
37) A fusion protein according to any one of the preceding 1-36, wherein the monovalent immunoglobulin or fragment thereof is a human IgG4, wherein one of the amino acid residues corresponding to amino acid residues 106 and 109 of the sequence of SEQ ID No: 14 has been substituted with an amino acid residue different from cysteine, and the other of the amino acid residues corresponding to amino acid residues 106 and 109 of the sequence of SEQ ID No: 14 has been deleted.
38) A fusion protein according to any one of the preceding 1-36, wherein the monovalent immunoglobulin or fragment thereof is a human IgG4, wherein the amino acid residue corresponding to amino acid residue 106 has been substituted with an amino acid residue different from cysteine, and the amino acid residue corresponding to amino acid residue 109 has been deleted.
39) A fusion protein according to any one of the preceding 1-36, wherein the monovalent immunoglobulin or fragment thereof is a human IgG4, wherein the amino acid residue corresponding to amino acid residue 106 has been deleted, and the amino acid residue corresponding to amino acid residue 109 has been substituted with an amino acid residue different from cysteine.
40) A fusion protein according to any one of the preceding 1-36, wherein the monovalent immunoglobulin or fragment thereof is a human IgG4, which is a human IgG4, wherein at least the amino acid residues corresponding to amino acid residues 106 to 109 of the C_{H} sequence of SEQ ID No: 14 has been deleted.
41) A fusion protein according to any one of the preceding 1-36, wherein the monovalent immunoglobulin or fragment thereof is a human IgG4, wherein at least the amino acid residues corresponding to amino acid residues 99 to 110 of the sequence of SEQ ID No: 14 has been deleted.
42) A fusion protein according to any one of the preceding 1-36, wherein the monovalent immunoglobulin or fragment thereof is a human IgG4, wherein the C_{H} region comprises the amino acid sequence of SEQ ID No: 16.
43) A fusion protein according to any one of the preceding 1-36, wherein the monovalent immunoglobulin or fragment thereof is a human IgG4, wherein the C_{H} region has been modified such that the entire hinge region has been deleted.
44) A fusion protein according to 1-43, wherein said fusion or linked protein has a plasma concentration above 10 mg/ml for more than 7 days when administered in vivo to a human being or to a SCID mouse at a dose of 4 mg/kg.
45) A fusion protein according to any of 1 to 43, wherein said fusion or linked protein has a half-life of at least 5 days when administered in vivo.
46) A fusion protein according to any one of 1 to 43, wherein said fusion or linked protein has a half-life of at least 5 and up to 21 days when administered in vivo to a human being or a SCID mouse.
47) A fusion protein according to any one of 1 to 46, wherein said fusion or linked protein is capable of binding to FcRn.
48) A fusion protein according to any one of 1-47, wherein the molecule linked to the monovalent immunoglobulin or fragment thereof is a small organic molecule.
49) A fusion protein according to any one of 1 to 47, wherein the cytokine or other protein in the fusion or linked protein is selected from the list of erythropoietin, thrombopoietin, interferon-alpha (2a and 2b), -beta (1b), -gamma, TNFR I (CD120a), TNFR II (CD120b), IL-1R type 1 (CD121a), IL-1R type 2 (CD121b), IL-2, IL2R (CD25), IL-2R-beta (CD123), IL-3, IL-4, IL-3R (CD123), IL-4R (CD124), IL-5R (CD125), IL-6R-alpha (CD126), -beta (CD130), IL-10, IL-11, IL-15BP, IL-15R, IL-20, IL-21, TCR variable chain, RANK, RANK-L, CTLA4, CXCR4R, CCR5R, TGF-beta1, -beta2, -beta3, G-CSF, GM-CSF, MIF-R (CD74), M-CSF-R (CD115), GM-CSFR (CD116), soluble FcgammaRI, sFcgammaRIIa, sFcgammaRIIb, sFcgammaRIIIa, sFcgammaRIIIb, sFcRn, sFcepsilonRI, sFcepsilonRIIa, sFcepsilonRIIb, sFcalphaI, Factor VII, Factor VIII, Factor IX, VEGF, VEGFxxxb, soluble Siglec-1, sSiglec-2, sSiglec-3, sSiglec-4, sSiglec-5, sSiglec-6, sSiglec-7, sSiglec-8, sSiglec-9, sSiglec-10, sSiglec-11, sSiglec-12, sSiglec-14, sSiglec-15.
50) A fusion protein according to any one of 1 to 49, wherein the cytokine or other protein in the fusion or linked protein is selected from the list of anti-psychotic drugs, anti-depressant drugs, anti-Parkinson drugs, anti-seizure agents, neuromuscular blocking drugs, anti-epileptic drugs, adrenocorticosteroids, insulin, proteins or enzymes involved in regulation of insulin, incretins (GIP and GLP-1) or drugs mimicking incretin action such as Exenatide and sitagliptin, thyroid hormones, growth hormone, ACTH, oestrogen, testosterone, anti-diuretic hormone, diuretics, all kinds of blood products such as heparin and EPO, beta-blocking agents, cytotoxic agents, anti-viral drugs, anti-bacterial agents, anti-fungal agents, anti-parasitic drugs, anti-coagulation drugs, anti-inflammatory drugs, anti-asthma drugs, and anti-COPD drugs.
51) A fusion protein according to 48, wherein the first molecule in the fusion protein is selected from the list of sildenafil citrate (Viagra), opiates, morphine, vitamins (such as vitamin C for conservation), hormones involved in pregnancy such as LH and FSH, hormones involved in sex changes, anti-conceptives, and antibodies
52) A fusion protein according to any one of the preceding 1-51, wherein the monovalent immunoglobulin or fragment thereof is selected from the list: single-chain Fv, dAb or domain antibody, nanobody, VHH, diabody, V-NAR, ScFab, CTL-4, tendamistat, 10^{th} fibronectin type 3 domain, neocarzinostatin, CBM4-2, Lipocalins, T-cell receptor, Protein A domain (protein Z), Im9, Designed AR proteins, Zinc finger, pVIII, Avian pancreatic polypeptide, GCN4, WW domain, Src homology domain 3 (SH3), Src homology domain 2, PDZ domains, TEM-1 □-lactamase, GFP, Thioredoxin, Staphylococcal nuclease, PHD-finger, CL-2, BPTI, APPI, HPSTI, Ecotin, LACI-D1, LDTI, MTI-II, Scorpion toxins, Insect defensin A peptide, EETI-II, Min-23, CBD, PBP, cytochrome b₅₆₂, Ldl receptor domain A, □-chrystallin, ubiquitin, transferrin, C-type lectin-like domain (the scaffolds are described in Table 1 of Binz et al. (2005) Nature Biotechnology, vol. 23, no. 10, page 1257-1268).

In further embodiments, the monovalent antibody according to the invention has been further modified e.g. in the CH2 and/or CH3 region, for example, to reduce the ability of the monovalent antibody to dimerize or to improve the pharmacokinetic profile, e.g. via improving the binding to FcRn.

Examples of such modifications include the following substitutions (reference is here made to IgG4 residues given in SEQ ID NO:16, but the same substitutions may be made in corresponding residues in other isotypes, such as IgG1. These corresponding residues may be found by simply alignment of the sequence): in the CH3 region: T234A, L236A, L236V, F273A, F273L, Y275A, E225A, K238A, K238T, D267A, L236E, L236G, F273D, F273T, Y275E, and in the CH2 region: T118Q, M296L, M120Y, S122T, T124E, N302A, T175A, E248A, N302A. Two or more of the above mentioned substitutions made combined to obtain the combined effects.

Thus, in one embodiment, the monovalent antibody comprises the CH3 region as set forth in SEQ ID NO: 16.

However, in another embodiment, the monovalent antibody comprises the CH3 region as set forth in SEQ ID NO: 16, but:
Glu (E) in position 225 has been replaced by Ala (A), and/or
Thr (T) in position 234 has been replaced by Ala (A), and/or
Leu (L) in position 236 has been replaced by Ala (A), Val (V), Glu (E) or Gly (G), and/or
Lys (K) in position 238 has been replaced by Ala (A), and/or
Asp (D) in position 267 has been replaced by Ala (A), and/or
Phe (F) in position 273 has been replaced by Ala (A) or Leu (L).
Tyr (Y) in position 275 has been replaced by Ala (A).

In another embodiment, the monovalent antibody comprises the CH3 region as set forth in SEQ ID NO: 16, but:
Glu (E) in position 225 has been replaced by Ala (A), and/or
Thr (T) in position 234 has been replaced by Ala (A), and/or
Leu (L) in position 236 has been replaced by Ala (A), Val (V), Glu (E) or Gly (G), and/or
Lys (K) in position 238 has been replaced by Ala (A), and/or
Asp (D) in position 267 has been replaced by Ala (A), and/or
Phe (F) in position 273 has been replaced by Asp (D) and Tyr (Y) in position 275 has been replaced by Glu (E).

In another embodiment, the monovalent antibody comprises the CH3 region as set forth in SEQ ID NO: 16, but:
Glu (E) in position 225 has been replaced by Ala (A), and/or
Thr (T) in position 234 has been replaced by Ala (A), and/or
Leu (L) in position 236 has been replaced by Ala (A), Val (V), Glu (E) or Gly (G), and/or
Lys (K) in position 238 has been replaced by Ala (A), and/or
Asp (D) in position 267 has been replaced by Ala (A), and/or
Phe (F) in position 273 has been replaced by Thr (T) and Tyr (Y) in position 275 has been replaced by Glu (E).

In one embodiment, the monovalent antibody comprises the CH2 region as set forth in SEQ ID NO: 16, but wherein Thr (T) in position 118 has been replaced by Gln (Q) and/or Met (M) in position 296 has been replaced by Leu (L).

In another embodiment, the monovalent antibody comprises the CH2 region as set forth in SEQ ID NO: 16, but wherein one, two or all three of the following substitutions have been made: Met (M) in position 120 has been replaced by Tyr (Y); Ser (S) in position 122 has been replaced by Thr (T); and Thr (T) in position 124 has been replaced by Glu (E).

In another embodiment, the monovalent antibody comprises the CH2 region as set forth in SEQ ID NO: 16, but wherein Asn (N) in position 302 has been replaced by Ala (A).

In a yet other embodiment, the monovalent antibody comprises the CH2 region as set forth in SEQ ID NO: 16, but wherein Asn (N) in position 302 has been replaced by Ala (A) and Thr (T) in position 175 has been replaced by Ala (A) and Glu (E) in position 248 has been replaced by Ala (A)

Preferred substitutions include: replacement of Leu (L) in position 236 by Val (V), replacement of Phe (F) in position 273 by Ala (A) and replacement of Tyr (Y) in position 275 by Ala (A).

The present invention also provides pharmaceutical compositions comprising the fusion proteins according to the invention.

The present invention also provides pharmaceutical compositions further comprising one or more pharmaceutically acceptable excipients, diluents or carriers.

The present invention also provides pharmaceutical compositions comprising fusion proteins and, wherein the composition further comprises one or more further therapeutic agents.

The present invention also provides fusion proteins, for use as a medicament.

The present invention also provides fusion proteins, for use in the treatment of cancer, psychosis, depression, Parkinsons disease, seizure, neuromuscular diseases, epilepsia, diabetes, bacterial or viral infections, fungus infections, coagulation disorders, asthma, COPD.

The present invention also provides fusion proteins, for use in the treatment of an inflammatory condition.

The present invention provides fusion proteins, for use in the treatment of an auto(immune) disorder.

The present invention also provides fusion proteins, for use in the treatment of a disorder involving undesired angiogenesis.

The present invention also provides the use of a fusion protein as a medicament.

The present invention also provides the use of a fusion protein in the preparation of a medicament for the treatment of a disease as defined above, wherein the treatment comprises administering one or more further therapeutic agents.

The present invention also provides the use of a fusion protein in a method of treating a disease or disorder as defined above, wherein said method comprises administering to a subject in need of such treatment a therapeutically effective amount of a fusion protein or a pharmaceutical composition comprising a fusion protein.

The present invention also provides the use of a fusion protein in a method of treatment, wherein the treatment comprises administering one or more further therapeutic agents.

The present invention also provides the use of a fusion protein as a diagnostic agent.

The present invention also provides a nucleic acid construct, encoding the fusion protein of the invention, wherein the fusion protein comprise two polypeptides fused by peptide bonds, optionally separated by a peptide linker.

The present invention also provides a nucleic acid construct, encoding the fusion protein, wherein said nucleic acid construct is an expression vector.

The present invention also provides a nucleic acid construct encoding the fusion protein of the invention, for use in gene therapy.

The present invention also provides a pharmaceutical composition which comprises the nucleic acid construct for gene therapy.

The present invention also provides a method for preparing a fusion protein according to the invention, wherein the first molecule is a cytokine or other polypeptide, said method comprising the following steps:
i) providing an expression system comprising a nucleotide encoding the polypeptide,
ii) providing an expression system comprising a nucleotide encoding the monovalent immunoglobulin or a fragment thereof,
iii) expressing said cytokine or other peptide, and said monovalent immunoglobulin
iv) recovering and purifying said expressed proteins
v) combining the purified proteins by covalent binding

The present invention also provides a method of preparing a fusion protein according to the invention, said method comprising:
a. providing a nucleic acid construct encoding a fusion protein of said cytokine or other peptide and a monovalent immunoglobulin or a fragment of a monovalent immunoglobulin according to the invention,
b. wherein said nucleotide sequence encoding said monovalent immunoglobulin or fragment thereof are operably linked together.
c. providing a cell expression system for producing said fusion protein;
d. producing said fusion protein by expressing said nucleic acid construct in cells of the cell expression system of ii)
e. recovering said expressed fusion protein from the media or cells

The present invention also provides a host cell comprising a nucleic acid according to the invention, as described above.

The present invention also provides a host cell, which host cell is a prokaryotic cell, such as an E. coli cell.

The present invention also provides a host cell, which host cell is a eukaryotic cell, such as a mammalian cell, insect, plant or a fungal cell.

The present invention also provides a non human transgenic animal comprising a nucleic acid construct according to the invention, as described above.

### DESCRIPTION OF FIGURES

FIG. 1: The CD20-specific antibodies 7D8-IgG1, 7D8-IgG4 and 7D8-HG were evaluated on non-reducing SDS-PAGE.

Lane 1: Marker SeaBlue plus2 prestained (Invitrogen BV, The Netherlands), Lane 2: internal control, Lane 3: 7D8-IgG1, Lane 4: 7D8-IgG4, and Lane 5: 7D8-HG.

FIG. 2: Extracted ion chromatogram for [M+3H]3+ and [M+2H]2+ ions (m/z 676.4 and 1014.1 respectively) eluting at 39.3 mins TIC time in the reduced CNBr/tryptic digest of 7D8-HG.

FIG. 3: The raw data obtained from nanospray-MS/MS analysis of the m/z signals consistent with a peptide covering amino acid residues 220 to 238 (²²⁰ VAPEFLGGPSVFLFPPKPK²³⁸) (SEQ ID NO: 54) from a reduced CNBr/tryptic digest of 7D8-HG.

FIGS. 4A and B: Interpretation of the raw data obtained from nanospray-MS/MS analysis of the m/z signals consistent with a peptide covering amino acid residues 220 to 238 (²²⁰VAPEFLGGPSVFLFPPKPK²³⁸) (SEQ ID NO: 54) from a reduced CNBr/tryptic digest of 7D8-HG. The sequences shown in FIG. 4B are given in SEQ ID NO: 55 and SEQ ID NO: 56. The highlighted sequence corresponds to amino acids 99-110 of SEQ ID NO: 14 which are deleted in SEQ ID NO: 16.

FIG. 5: The CD20-specific antibodies 7D8-IgG1, 7D8-IgG4 and 7D8-HG were evaluated on their binding to CD20 transfected cells.

FIG. 6: The CD20-specific antibodies 7D8-IgG1, 7D8-IgG4 and 7D8-HG were coated on an ELISA plate (concentration range as indicated on x-axis). C1q binding (2 μg/ml) was evaluated.

FIG. 7: A) Daudi cells were pre-incubated with a concentration range of the CD20-specific antibodies for 10 minutes, before NHS was added. Forty-five minutes after induction of CDC, cells were resuspended in PI solution. Cell lysis (number of PI-positive cells) was measured by flow cytometry. Data show the Mean Fluorescence intensity of the PI-positive (dead) cells.

B) To evaluate the role of complement in the lysis measured, heat-inactivated serum (serum ΔT) was added to cells incubated with 10 μg antistof. Data show the mean fluorescence intensity of the PI-positive (dead) cells.

FIG. 8: The hingeless IgG4 antibody directed against Bet v 1 (Betv1-HG) was tested on non-reducing SDS-PAGE.

Lane 1: Marker SeaBlue plus2 prestained (Invitrogen BV, The Netherlands), lane 2: internal control, lane 3: BetV1-HG, lane 4: IgG1 control.

FIG. 9: Gelfiltration of Betv1-HG (hingeless IgG4 anti-Bet v 1). Conditioned medium from HEK cells containing hingeless rIgG4 Betv1-HG was fractionated on a Superdex200 column. A total 1 μg of Betv1-HG was applied to the column. In the fractions, Bet v 1 specific IgG () was measured by incubating 10 μl of each fraction in the Bet v 1 binding test. The results are expressed as percentage of radiolabeled Bet v 1 binding relative to the amount added. The dashed curve represents the elution of purified Betv1-IgG4 (10 μg), which was followed on the HPLC by measuring the absorption at 214 nm (A214 nm).

FIG. 10: The binding of Betv1-IgG1, Betv1-IgG4 and Betv1-HG was examined in an radio immuno assay. The binding of ¹²⁵I-labelled Bet v1 to serial dilutions of the antibodies bound to Protein G Sepharose was examined.

FIG. 11: The ability of Betv1-IgG1, Betv1-IgG4 and Betv1-HG to crosslink Sepharose bound Bet v 1 to radiolabelled Bet v 1 was examined in an radio immuno assay. The binding of ¹²⁵I-labelled Bet v1 to serial dilutions of the antibodies bound to Bet v 1 Sepharose was examined.

FIG. 12: Semilogarithmic plot of the mouse plasma concentrations of 7D8-HG in comparison with normal 7D8-IgG4, intact 7D8-IgG1, 7D8-IgG1, F(ab′)₂ and 7D8-IgG1 Fab fragments after intravenous administration of 100 μg per mouse.

FIG. 13: Logarithmic plot of the plasma clearance rates as dose/area under the curve calculated from the concentration-time curves (D/AUC). The data represent individual mice and are expressed in ml.day⁻¹.kg⁻¹.

FIG. 14: Dose-response curves showing the inhibition of EGF-induced EGFr phosphorylation in A431 cells by anti-EGFr mAb 2F8-HG, compared with 2F8-IgG4 and 2F8-Fab fragments. The upper panel shows the inhibition curves in serum-deprived medium, the middle and lower panels the inhibition when IVIG was added to the medium at a concentration of 100 μg/ml and 1000 μg/ml, respectively. The y-axis represents Phosphorylated EGFr as detected with an anti-phospho-tyrosine mAb and is expressed in time-resolved fluorescence units (TRF units). On the x-axis, the mAb concentration in μg/ml. Data points are mean and SEM of 4 replicates.

FIG. 15: A semilogarithmic plot of the concentrations in time. The initial plasma concentrations were all in the order of 100 μg/ml, which is consistent with an initial distribution into the plasma compartment of the mice. The clearance of the hingeless IgG4 variant was only slightly faster than that of normal IgG4. Importantly, the clearance of the hingeless variant was much slower than that of F(ab′)₂ fragments, which have a comparable molecular size.

This experiment indicates that the Fc-part has a favorable effect on the plasma residence time in mice having a normal immune system and provides an indication of a functional interaction with the neonatal Fc receptor (FcRn) also in the presence of endogenous IgG.

FIG. 16: The binding of 2F8-HG to a coat of EGFr protein was compared in an ELISA to that of 2F8-IgG4, 2F8-IgG1 and Fab fragments of 2F8-IgG1, in the presence of polyclonal human IgG (IVIG) at a concentration of 100 μg/ml.

FIG. 17: The induction of ADCC by 2F8-HG was compared to that by 2F8-IgG1 and 2F8-IgG4. A431 cells were used as target cells and human peripheral blood mononuclear cells as effector cells

FIG. 18: Sequence of primers used in the Examples.

FIG. 19: Sequences of primers used in the Examples.

FIG. 20: Clearance of 7D8 variants in IVIG supplemented SCID mice. The figure shows in the upper panel semi-logarithmic plots of the concentrations of the mAb 7D8 variants in time and in the lower panel the total human IgG concentrations.

FIG. 21: Clearance with 7D8 variants in FcRn −/− mice vs wild type mice. The figure shows a semi-logarithmic plot of the concentrations in time. The initial plasma concentrations were all in the order of 100 μg/ml, which is consistent with an initial distribution in the plasma compartment of the mice. The hingeless IgG4 variant (7D8-HG), normal human IgG4 (7D8-IgG4) and F(ab′)₂ fragments from 7D8 IgG1 (7D8-G1-F(ab′)₂) were compared in the model.

FIG. 22: DU-145 cells were cultured and incubated with a serial dilution of (A) cMet-Fab, cMet-Fab and IVIG, cMet-Fab and HGF, cMet-Fab and IVIG and HGF (B) cMet-HG, cMet-HG and IVIG, cMet-HG and HGF, cMet-HG and IVIG and HGF. Scattering was observed double-blinded (scored by 14 people) by microscope after 48 h and the averaged score±SEM is plotted.

FIG. 23: DU-145 cells were cultured and incubated with 10 μg/ml of (A) cMet-Fab, cMet-Fab and IVIG, cMet-Fab and HGF, cMet-Fab and IVIG and HGF (B) cMet-HG, cMet-HG and IVIG, cMet-HG and HGF, cMet-HG and IVIG and HGF. Scattering was observed double-blinded (scored by 14 people) by microscope after 48 h. cMet-Fab with or without IVIG and cMet-HG pre-incubated with IVIG significantly inhibited the HGF induced scattering. For statistical analysis a two-tailed Wilcoxon signed ranked test was done with a hypothetical median value of 3 (maximal scattering).

FIG. 24: Extracts prepared from A549 cells incubated with cMet-HG (lane 1), cMet-HG and IVIG (lane 2), cMet-HG and HGF (lane 3), cMet-HG, IVIG and HGF (lane 4), cMet-IgG1 (lane 5), cMet-IgG1 and IVIG (lane 6) were resolved by SDS-PAGE on a 4-20% Tris-HCl Criterion Precast gel and Western blotting on a nitrocellulose membrane. The membrane was incubated over night at 4° C. with anti-phospho-Met(pYpYpY 1230 1234 1235)-rabbit IgG, (Abcam, ab5662). After washing with TBST, the secondary antibodies, goat-anti-rabbit-HRP, Cell Signalling, 7074 in blocking reagent were incubated for 60 min. at room temperature on a roller bank. The membrane was washed 6 times with TBST. Finally the bands were developed with Luminol Echancer stop solution and analyzed on a Lumiimager. The Western blot shows a 169 Kd band indicating phospho-Met(pYpYpY 1230 1234 1235).

FIG. 25: Starting concentration of addition of HuMax-CD4 or Fab fragments of HuMax-CD4 to the in vitro HIV-1 neutralization assay. The IC50 values of inhibition by HuMax-CD4 and Fab fragments of HuMax-CD4 are calculated by a 4 parameter logistic curve fit and indicated for each of the virus constructs.

FIG. 26: The % human T cells, % murine cells, and % CD4 and % CD8 cells, and the ratio CD4/CD8 of the individual PBMC reconstituted mice treated intraperitoneally with HuMax-CD4, IgG control or non treated, and infected with HIV-1.

FIG. 27: The inhibition curves of HuMax-CD4 and the Fab fragments of HuMax-CD4 of the infection of several strains of HIV-1 of CD4-CCR5 or CD4-CXCR4 positive cells measured by luciferase activity (mean of triplicate measurements).

FIG. 28: The plasma HuMax-CD4 concentrations in time of the individual PBMC reconstituted mice treated intraperitoneally with HuMax-CD4, or non treated, and infected with HIV-1.

FIG. 29: The measured HIV-1 RNA copies in time of the individual PBMC reconstituted mice treated intraperitoneally with HuMax-CD4, of IgG control or non treated, and infected with HIV-1.

FIG. 30: Percentage of molecules present as monomers for each HG mutant tested using non-covalent nano-electrospray mass spectrometry. HG mutant samples were prepared in aqueous 50 mM ammonium acetate solutions at a concentration of 1 μM.

FIG. 31: NativePAGE™ Novex® Bis-Tris gel electrophoresis of CH3 mutants compared to 2F8-HG (WT) and R277K HG mutant control.

FIG. 32: The binding of 2F8-HG and CH3 mutants 2F8-HG-T234A and 2F8-HG-L236V was tested in EGFR ELISA in the presence and absence of polyclonal human IgG.

FIG. 33: The binding of 2F8-HG and CH3 mutants 2F8-HG-L236A and 2F8-HG-Y275A was tested in EGFR ELISA in the presence and absence of polyclonal human IgG.

FIG. 34: Dose-response curves showing the inhibition of EGF-induced EGFr phosphorylation in A431 cells by anti-EGFr 2F8-HG (WT) and CH3 mutants thereof.

FIG. 35: Percentage molecules present as monomers at different molar concentrations of CH3 mutants compared to 2F8-HG (WT) and R277K. The Table shows EC50 values of monomer to dimer conversion, calculated for each CH3 mutant and 2F8-HG (WT) based on the curves presented in the figure.

### DETAILED DESCRIPTION OF THE SEQUENCE LISTING

SEQ ID No: 1: The nucleic acid sequence of C_{L} kappa of human Ig

SEQ ID No: 2: The amino acid sequence of the kappa light chain of human Ig

SEQ ID No: 3: The nucleic acid sequence of C_{L} lambda of human Ig

SEQ ID No: 4: The amino acid sequence of the lambda light chain of human Ig

SEQ ID No: 5: The nucleic acid sequence of the V_{H} region of HuMab-7D8

SEQ ID No: 6: The amino acid sequence of the V_{H} region of HuMab-7D8

SEQ ID No: 7: The nucleic acid sequence of the V_{H} region of mouse anti-Betv-1

SEQ ID No: 8: The amino acid sequence for the V_{H} region of mouse anti-Betv-1

SEQ ID No: 9: The nucleic acid sequence of the V_{L} region of HuMab-7D8

SEQ ID No: 10: The amino acid sequence of the V_{L} region of HuMab-7D8

SEQ ID No: 11: The nucleic acid sequence of the V_{L} region of mouse anti-Betv1

SEQ ID No: 12: The amino acid sequence of the V_{L} region of mouse anti-Betv1

SEQ ID No: 13: The nucleic acid sequence of the wildtype C_{H} region of human IgG4

SEQ ID No: 14: The amino acid sequence of the wildtype CH region of human IgG4.

Sequences in italics represent the CH1 region, highlighted sequences represent the hinge region, regular sequences represent the CH2 region and underlined sequences represent the CH3 region.

SEQ ID No: 15: The nucleic acid sequence of the CH region of human IgG4 (SEQ ID No: 13) mutated in positions 714 and 722

SEQ ID No: 16: The amino acid sequence of the hingeless CH region of a human IgG4

SEQ ID NO: 17: The amino acid sequence of the lambda chain constant human (accession number S25751)

SEQ ID NO: 18: The amino acid sequence of the kappa chain constant human (accession number P01834)

SEQ ID NO: 19: The amino acid sequence of IgG1 constant region (accession number P01857). Sequences in italics represent the CH1 region, highlighted sequences represent the hinge region, regular sequences represent the CH2 region and underlined sequences represent the CH3 region

SEQ ID NO: 20: The amino acid sequence of the IgG2 constant region (accession number P01859). Sequences in italics represent the CH1 region, highlighted sequences represent the hinge region, regular sequences represent the CH2 region and underlined sequences represent the CH3 region

SEQ ID NO: 21: The amino acid sequence of the IgG3 constant region (accession number A23511). Sequences in italics represent the CH1 region, highlighted sequences represent the hinge region, regular sequences represent the CH2 region and underlined sequences represent the CH3 region

SEQ ID NOs: 22 to 53 show oligonucleotide primers used for preparation of DNA constructs SEQ ID NO: 54: A peptide of a hingeless IgG4

SEQ ID NO: 55: A portion of the constant region of IgG4

SEQ ID NO: 56: A portion of the constant region of a hingeless IgG4

### DETAILED DESCRIPTION OF THE INVENTION

In order that the present invention may be more readily understood, certain terms are first defined. Additional definitions are set forth throughout the detailed description.

The term “antibody” as referred to herein includes whole antibody molecules, antigen binding fragments, monovalent antibodies, and single chains thereof. Antibody molecules belong to a family of plasma proteins called immunoglobulins, whose basic building block, the immunoglobulin fold or domain, is used in various forms in many molecules of the immune system and other biological recognition systems. Native antibodies and immunoglobulins are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies between the heavy chains of different immunoglobulin isotypes. Each heavy and light chain may also have regularly spaced intrachain disulfide bridges. Each light chain is comprised of a light chain variable region (abbreviated herein as V_{L}) and a light chain constant region (abbreviated herein as C_{L}). Each heavy chain is comprised of a heavy chain variable region (V_{H}) and a heavy chain constant region (C_{H}) consisting of three domaina, C_{H}1, C_{H}2 and C_{H}3, and the hinge region). The three CH domains and the hinge region have been indicated for IgG1, IgG2, IgG3 and IgG4 in SEQ ID NO: 19, 20, 21 and 14, respectively (see below The constant domain of the light chain is aligned with the first constant domain (C_{H}1) of the heavy chain, and the light chain variable domain is aligned with the variable domain of the heavy chain forming what is known as the “Fab fragment”. C_{H}1 and C_{H}2 of the heavy chain are separated form each other by the socalled hinge region, which allows the Fab “arms” of the antibody molecule to swing to some degree. The hinge region normally comprises one or more cysteine residues, which are capable of forming disulphide bridges with the cysteine residues of the hinge region of the other heavy chain in the antibody molecule.

The term “monovalent immunoglobulin” as referred to herein means a monovalent antibody or a fragment of a monovalent antibody, which exists in monomeric form in vivo or in the presence of polyclonal human IgG.

The variable regions of the heavy and light chains contain a binding domain that interacts with an antigen. The constant regions of the antibodies may mediate the binding of the immunoglobulin to host tissues or factors, including various cells of the immune system (for instance effector cells) and the first component (C1q) of the classical complement system Depending on the amino acid sequences of the constant domain of their heavy chains, immunoglobulins can be assigned to different classes. There are at least five (5) major classes of immunoglobulins: IgA, IgD, IgE, IgG and IgM, and several of these may be further divided into subclasses (isotypes), for instance IgG1, IgG2, IgG3 and IgG4; IgA1 and IgA2. The genes for the heavy chains constant domains that correspond to the different classes of immunoglobulins are called alpha (α), delta (δ), epsilon (ε), gamma (γ) and mu (μ), respectively. Immunoglobulin subclasses are encoded by different genes such as γ1, γ2, γ3 and γ4. The genes for the light chains of antibodies are assigned to one of two clearly distinct types, called kappa (κ) and lambda (λ), based on the amino sequences of their constant domain. The subunit structures and three-dimensional configurations of different classes of immunoglobulins are well known. Distinct allotypes of immunoglobulins exist within the human population such as G1 m(a), G1 m(x), G1 m(f) and G1 m(z) for IgG1 heavy chain and Km1, Km1,2 and Km3 for the kappa light chain. These allotypes differ at distinct amino acids in their region encoding the constant regions.

The term antibody also encompasses “derivatives” of antibodies, wherein one or more of the amino acid residues have been derivatised, for instance by acylation or glycosylation, without significantly affecting or altering the binding characteristics of the antibody containing the amino acid sequences.

In the context of the present invention, a derivative of a monovalent antibody may for instance be a monovalent antibody, in which one or more of the amino acid residues of the monovalent antibody have been chemically modified (for instance by alkylation, acylation, ester formation, or amide formation) or associated with one or more non-amino acid organic and/or inorganic atomic or molecular substituents (for instance a polyethylene glycol (PEG) group, a lipophilic substituent (which optionally may be linked to the amino acid sequence of the peptide by a spacer residue or group such as β-alanine, γ-aminobutyric acid (GABA), L/D-glutamic acid, succinic acid, and the like), a fluorophore, biotin, a radionuclide, etc.) and may also or alternatively comprise non-essential, non-naturally occurring, and/or non-L amino acid residues, unless otherwise stated or contradicted by context (however, it should again be recognized that such derivatives may, in and of themselves, be considered independent features of the present invention and inclusion of such molecules within the meaning of peptide is done for the sake of convenience in describing the present invention rather than to imply any sort of equivalence between naked peptides and such derivatives). Non-limiting examples of such amino acid residues include for instance 2-aminoadipic acid, 3-aminoadipic acid, β-alanine, β-aminopropionic acid, 2-aminobutyric acid, 4-aminobutyric acid, 6-aminocaproic acid, 2-aminoheptanoic acid, 2-aminoisobutyric acid, 3-aminoisobutyric acid, 2-aminopimelic acid, 2,4-diaminobutyric acid, desmosine, 2,2′-diaminopimelic acid, 2,3-diaminopropionic acid, N-ethylglycine, N-ethylasparagine, hydroxylysine, allohydroxylysine, 3-hydroxyproline, 4-hydroxyproline, isodesmosine, alloisoleucine, N-methylglycine, N-methylisoleucine, 6-N-methyllysine, N-methylvaline, norvaline, norleucine, ornithine, and statine halogenated amino acids.

The in vivo half-life of the antibodies may for instance be improved by modifying the salvage receptor epitope of the Ig constant domain or an Ig-like constant domain such that the molecule does not comprise an intact C_{H}2 domain or an intact Ig Fc region, cf. U.S. Pat. No. 6,121,022 and U.S. Pat. No. 6,194,551. The in vivo half-life may be furthermore increased by making mutations in the Fc region, for instance by substituting threonine for leucine at the position corresponding to position 252 of an intact antibody molecule, threonine for serine at the position corresponding to position 254 of an intact antibody molecule, or threonine for phenylalanine at the position corresponding to position 256 of an intact antibody molecule, cf. U.S. Pat. No. 6,277,375. Furthermore, antibodies, and particularly Fab or other fragments, may be pegylated to increase the half-life. This can be carried out by pegylation reactions known in the art, as described, for example, in Focus on Growth Factors 3, 4-10 (1992), EP 154 316 and EP 401 384.

Mutations may also be introduced randomly along all or part of an antibody coding sequence, such as by saturation mutagenesis, and the resulting modified antibodies can be screened for binding activity and/or other characteristics.

The term “antibody derivatives” refers to any modified form of the antibody, for instance a conjugate of the antibody and another agent or antibody.

The term “antigen-binding portion” or “antigen-binding domain” of an antibody, such as a monovalent antibody, as used herein, refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding portion” of an antibody include
(i) a Fab or Fab′ fragment, a monovalent fragment consisting of the V_{L}, V_{H}, C_{L} and C_{H1} domains;
(ii) F(ab′)₂ fragment, a bivalent fragment comprising two Fab′ fragments linked by a disulfide bridge at the hinge region;
(iii) a Fd fragment consisting essentially of the V_{H} and C_{H1} domains;
(iv) a Fv fragment consisting essentially of the V_{L} and V_{H} domains of a single arm of an antibody,
(v) a dAb fragment (Ward et al., Nature 341, 544-546 (1989)), which consists essentially of a V_{H} domain;
(vi) an isolated complementarity determining region (CDR), and
(vii) a combination of two or more isolated CDRs which may optionally be joined by a synthetic linker.

Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain antibodies or single chain Fv (scFv), see for instance Bird et al., Science 242, 423-426 (1988) and Huston et al., PNAS USA 85, 5879-5883 (1988)). Such single chain antibodies are encompassed within the term antibody unless otherwise noted or clearly indicated by context.

A further example is antigen-binding-domain immunoglobulin fusion proteins comprising an antigen-binding domain polypeptide that is fused to
(i) an immunoglobulin hinge region polypeptide,
(ii) an immunoglobulin heavy chain C_{H}2 constant region fused to the hinge region, and
(iii) an immunoglobulin heavy chain C_{H}3 constant region fused to the C_{H}2 constant region.

The antigen-binding domain polypeptide may be a heavy chain variable region or a light chain variable region, a scFv or any other polypeptide capable of binding specifically to the antigen. Such binding-domain immunoglobulin fusion proteins are further disclosed in US 2003/0118592 and US 2003/0133939. These antibody fragments are obtained using conventional techniques known to those with skill in the art, and the fragments are screened for utility in the same manner as are intact antibodies.

The term “antibody half-molecule” is used herein to mean an antibody molecule as described above, but comprising no more than one light chain and no more than one heavy chain, and which exists in water solutions as a heterodimer of said single light and single heavy chain. Such antibody is by nature monovalent as only one antigen-binding portion is present.

The term “conservative sequence modifications” in the context of nucleotide or amino acid sequences are modifications of nucleotide(s) and amino acid(s), respectively), which do not significantly affect or alter the binding characteristics of the antibody encoded by the nucleotide sequence or containing the amino acid sequence. Such conservative sequence modifications include nucleotide and amino acid substitutions, additions and deletions. Modifications may be introduced into the sequences by standard techniques known in the art, such as site-directed mutagenesis and PCR-mediated mutagenesis. Conservative amino acid substitutions include ones in which the amino acid residue is replaced with an amino acid residue having a similar side chain. Families of amino acid residues having similar side chains have been defined in the art. These families include amino acids with basic side chains (for instance lysine, arginine, histidine), acidic side chains (for instance aspartic acid, glutamic acid), uncharged polar side chains (for instance glycine, asparagine, glutamine, serine, threonine, tyrosine, cysteine, tryptophan), nonpolar side chains (for instance alanine, valine, leucine, isoleucine, proline, phenylalanine, methionine), beta-branched side chains (for instance threonine, valine, isoleucine) and aromatic side chains (for instance tyrosine, phenylalanine, tryptophan, histidine). Thus, a predicted nonessential amino acid residue in a human antibody specific for a certain antigen may be replaced with another amino acid residue from the same side chain family.

As used herein, a human antibody is “derived from” a particular germline sequence if the antibody is obtained from a system using human immunoglobulin sequences, for instance by immunizing a transgenic mouse carrying human immunoglobulin genes or by screening a human immunoglobulin gene library, and wherein the variable gene encoded region (not including the heavy or light chain CDR3) of the selected human antibody is at least 90%, more preferably at least 95%, even more preferably at least 96%, 97%, 98%, or 99% identical in nucleic acid sequence to the germline immunoglobulin gene. Typically, a human antibody derived from a particular human germline sequence will display no more than 10 amino acid differences, more preferably, no more than 5, or even more preferably, no more than 4, 3, 2, or 1 amino acid difference from the amino acid sequence encoded by the germline immunoglobulin gene.

The term “epitope” means a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents.

The term “discontinuous epitope”, as used herein, means a conformational epitope on a protein antigen which is formed from at least two separate regions in the primary sequence of the protein.

The term “fragment of a monovalent immunoglobulin” is a fragment which at least comprises the CH2 and CH3 domains.

The term “fusion protein” as referred to herein, describe a molecule comprising a first molecule which may in non limiting example be a polypeptide, a peptide mimetic, a cytokine or a small organic molecule, and a second molecule which is a monovalent immunoglobulin, or a fragment of a monovalent immunoglobulin, wherein the first and second molecule may be fused together by peptide bonding, or fused together by other covalent bonding. Linker sequences or different types of chemical linkers may be used as spacers and/or mediators of the binding between the two fusion partners.

Chemical linker technology has been well known in the art for many years, as exemplified by the book Hermanson, G. T. (1996). Bioconjugate Techniques, Academic Press, and chemical linkers may be purchased from e.g. Pierce (Rockford P.O. Box 117, IL 61105, USA), therefore it is evident that the person skilled in the art, would readily be able to use this technology to crosslink two polypeptides of the invention, or to crosslink e.g. a small organic molecule with a polypeptide.

For nucleotide and amino acid sequences, the term “homology” indicates the degree of identity between two nucleic acid or amino acid sequences when optimally aligned and compared with appropriate insertions or deletions. Alternatively, substantial homology exists when the DNA segments will hybridize under selective hybridization conditions, to the complement of the strand.

The percent identity between two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, for instance as described in the following. The percent identity between two nucleotide sequences may be determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (Comput. Appl. Biosci., 4, 11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48, 444-453 (1970)) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

The term “host cell” (or “recombinant host cell”), as used herein, is intended to refer to a cell into which a recombinant expression vector has been introduced. It should be understood that such terms are intended to refer not only to the particular subject cell but also to the progeny of such a cell. Because certain modifications may occur in succeeding generations due to either mutation or environmental influences, such progeny may not, in fact, be identical to the parent cell, but are still included within the scope of the term “host cell” as used herein. Recombinant host cells include, for example, transfectomas, such as transfected CHO cells, NS/0 cells, and lymphocytic cells. The term “host cell” in singular form may also denote a culture of a specific kind of host cell.

The term “human antibody”, as used herein, is intended to include antibodies having variable and constant regions derived from human germline immunoglobulin sequences. The human antibodies of the invention may include amino acid residues not encoded by human germline immunoglobulin sequences (for instance mutations introduced by random or site-specific mutagenesis in vitro or by somatic mutation in vivo). However, the term “human antibody”, as used herein, is not intended to include antibodies in which CDR1 or CDR2 sequences derived from the germline of another mammalian species, such as a mouse, or the CDR3 region derived from an antibody from another species, such as mouse, have been grafted onto human framework sequences.

The term “K_{D}” (M), as used herein, refers to the dissociation equilibrium constant of a particular antibody-antigen interaction.

The terms “monoclonal antibody” or “monoclonal antibody composition” as used herein refer to a preparation of antibody molecules of single molecular composition. A monoclonal antibody composition displays a single binding specificity and affinity for a particular epitope. Accordingly, the term “human monoclonal antibody” refers to antibodies displaying a single binding specificity which have variable and constant regions derived from human germline immunoglobulin sequences.

The term “monovalent antibody” means in the present context that an antibody molecule is capable of binding a single molecule of the antigen, and thus is not able of antigen crosslinking.

The term “nucleic acid”, nucleic acid construct” or “nucleic acid molecule”, as used herein, is intended to include DNA molecules and RNA molecules. A nucleic acid molecule may be single-stranded or double-stranded.

The term “isolated nucleic acid”, “isolated nucleic acid construct” or “isolated nucleic acid molecule”, as used herein in reference to nucleic acids encoding antibodies, or fragments thereof is intended to refer to a nucleic acid molecule in which the nucleotide sequences encoding the intact antibody, or fragment thereof, are free of other nucleotide sequences. A nucleic acid may be isolated or rendered substantially pure, when purified away from other cellular components or other contaminants, for instance other cellular nucleic acids or proteins, by standard techniques, including alkaline/SDS treatment, CsCl banding, column chromatography, agarose gel electrophoresis and others well known in the art. See, F. Ausubel, et al., ed. Current Protocols in Molecular Biology, Greene Publishing and Wiley Interscience, New York (1987).

A nucleic acid is “operably linked” when it is placed into a functional relationship with another nucleic acid sequence. For instance, a promoter or enhancer is operably linked to a coding sequence if it affects the transcription of the sequence. For switch sequences, operably linked indicates that the sequences are capable of effecting switch recombination. When reference is made to “physiological condition” it is meant a condition that exists in vivo, within the organism, or an in vivo condition which is recreated by fully or partially mimicking said in vivo condition, for example a water solution with an equivalent osmotic value as the blood.

The term “recombinant human antibody”, as used herein, includes all human antibodies that are prepared, expressed, created or isolated by recombinant means, such as for instance (a) antibodies isolated from an animal (for instance a mouse) that is transgenic or transchromosomal for human immunoglobulin genes or a hybridoma prepared therefrom, (b) antibodies isolated from a host cell transformed to express the antibody, for instance from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable and constant regions derived from human germline immunoglobulin sequences. Such recombinant human antibodies may be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

As used herein, “specific binding” refers to the binding of an antibody, or antigen-binding fragment thereof, to a predetermined antigen. Typically, the antibody binds with an affinity corresponding to a K_{D} of about 10⁻⁷ M or less, such as about 10⁻⁸ M or less, such as about 10⁻⁹ M or less, about 10⁻¹⁰ M or less, or about 10⁻¹¹ M or even less, when measured for instance using sulfon plasmon resonance on BIAcore or as apparent affinities based on IC₅₀ values in FACS or ELISA, and binds to the predetermined antigen with an affinity corresponding to a K_{D} that is at least ten-fold lower, such as at least 100 fold lower, for instance at least 1000 fold lower, such as at least 10,000 fold lower, for instance at least 100,000 fold lower than its affinity for binding to a non-specific antigen (e.g., BSA, casein) other than the predetermined antigen or a closely-related antigen. The amount with which the affinity is lower is dependent on the K_{D} of the antigen binding peptide, so that when the K_{D} of the antigen binding peptide is very low (that is, the antigen binding peptide is highly specific), then the amount with which the affinity for the antigen is lower than the affinity for a non-specific antigen may be at least 10,000 fold.

As used herein, the term “subject” includes any human or non-human animal. The term “non-human animal” includes all vertebrates, for instance mammals and non-mammals, such as non-human primates, sheep, goat, dog, cow, mouse, rat, rabbit, chickens, amphibians, reptiles, etc.

When reference is made to a “therapeutically” effective dosage or a “therapeutically effective amount”, it should be taken to mean a dosage or amount effective to achieve a desired therapeutic result over a certain period of time. A therapeutically effective dosage of a monovalent antibody of the invention will of course vary with the target of the antibody and may also vary according to factors such as the disease state, age, sex, and weight of the individual, and the ability of the monovalent antibody to elicit a desired response in the individual. A therapeutically effective dosage or amount may also be one in which any toxic or detrimental effects of the monovalent antibody are outweighed by the therapeutically beneficial effects.

The terms “transgenic, non-human animal” refers to a non-human animal having a genome comprising one or more human heavy and/or light chain transgenes or transchromosomes (either integrated or non-integrated into the animal's natural genomic DNA) and which is capable of expressing human antibodies. For example, a transgenic mouse can have a human light chain transgene and either a human heavy chain transgene or human heavy chain transchromosome, such that the mouse produces human antibodies when immunized with an antigen and/or cells expressing an antigen. The human heavy chain transgene can be integrated into the chromosomal DNA of the mouse, as is the case for transgenic, for instance HuMAb mice, such as HCo7 or HCo12 mice, or the human heavy chain transgene can be maintained extrachromosomally, as is the case for transchromosomal KM mice as described in WO 02/43478. Such transgenic and transchromosomal mice are capable of producing multiple classes and isotypes of monovalent antibodies to a given antigen (for instance IgM, IgG, IgA and/or IgE) by undergoing V-D-J recombination and isotype switching.

The term “transfectoma”, as used herein, includes recombinant eukaryotic host cells expressing the antibody, such Chinese hamster ovary (CHO) cells, NS/0 cells, HEK293 cells, plant cells, or fungi, including yeast cells.

The term “treatment” or “treating” or “treat” means easing, ameliorating, or eradicating (curing) symptoms or disease states.

The term “valence of an antibody” means the maximum number of antigenic determinates with which the antibody can react. For example IgG antibodies contain two Fab regions and can bind two molecules of antigen or two identical sites on the same particle, and thus have a valence of two.

The term “vector”, as used herein, is intended to refer to a nucleic acid molecule capable of transporting and inducing replication of another nucleic acid to which it has been linked. One type of vector is a “plasmid”, which refers to a circular double stranded DNA loop into which additional DNA segments may be ligated. Another type of vector is a viral vector, wherein additional DNA or RNA segments may be ligated into the viral genome. Certain vectors are capable of autonomous replication in a host cell into which they are introduced (for instance bacterial vectors having a bacterial origin of replication and episomal mammalian vectors). Other vectors (for instance non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (for instance replication defective retroviruses, adenoviruses and adeno-associated viruses), which serve equivalent functions.

A number of references are made herein to a water solution or physiological conditions. When reference is made to “water solution” it is meant solution of any chemical matter in water, for example a salt solution, such as phosphate buffered saline (PBS). A water solution may be designed for the purpose and contain a number of different chemical matters, or it may be a natural body fluid, for example the blood.

Five different classes of immunoglobulins exist, i.e. IgM, IgD, IgG, IgA and IgE, and these classes can be distinguished by their C regions.

Within the IgG class of antibodies several subclasses exist, i.e. in human IgG1, IgG2, IgG3, and IgG4 (Jefferis, R. 1990. Molecular structure of human IgG subclasses. In The human IgG subclasses. F. Shakib, ed. Pergamon Press, Oxford, p. 15). Each IgG heavy chain is composed of structurally related peptide sequences (i.e. variable and constant region domains) that are encoded by distinct gene segments or exons. The hinge region linking the CH1 and CH2 domain is encoded by a separate exon. Each of the four IgG subclass heavy chains may be expressed in combination with either kappa or lambda light chains to give an essentially symmetrical molecule composed of two identical heavy chains and two identical kappa or lambda light chains. Comparison within the heavy chain defines the CH1, CH2 and CH3 homology regions. Comparisons between like homology regions of each of the four subclasses reveals >95% sequence identity (Jefferis, R. 1990. F. Shakib, ed. Pergamon Press, Oxford, p. 15). The sequence between the CH1 and CH2 domains is referred to as the hinge region because it allows molecular flexibility. The CH3 domains are paired and the non-covalent interactions are sufficient for the IgG molecule to maintain its structural integrity following reduction of the inter-heavy chain disulphide bridges under mild conditions. CH3 domain pairing is compact and similar to pairing in the Fab, with a nearly exact dyad between the two domains (Saphire, et al., 2002. J Mol Biol 319:9). This is in contrast to the CH2 domains, which do not associate closely and their contact is primarily mediated by the two carbohydrate chains attached to the Asn297 residues (Saphire, et al., 2002. J Mol Biol 319:9).

The characteristic IgG structure in which two heavy-light chain heterodimers are linked is thus maintained by the inter-heavy chain disulphide bridges of the hinge region and the non-covalent interactions of the CH3 domains.

The interaction in the CH3 region has shown to be important in IgG1. Ig half-molecules, which have a dimeric configuration consisting of only one light chain and only one heavy chain, have been described as the result of rare deletions in human and murine plasmacytomas. Several patients suffering from extramedullary soft-tissue plasmacytoma, Waldenström macroglobulinemia, plasma cell leukemia and multiple myeloma, excreted IgG half molecules into their urine. Half-molecules were also found to be present in their serum. Studies on the biochemical nature of these half-molecules showed that they consist of IgG1 molecules in which the heavy chain C_{H}1, hinge and C_{H}2 regions appeared normal, whereas deletions were found in the C_{H}3 region (already in patent application; page 3).

We have shown that removal of the hinge region in IgG4 results in the formation of monovalent antibodies in which the linkage between the two heavy-light chain heterodimers is lost or diminished. Consequently, changes in hinge region disulphide bridges of other IgG subclasses alone or in combination with mutations in the CH3 domain interactions may result in the formation of monovalent antibodies for these other subclasses as well. It is well within the capability of the skilled artisan to use the intimate knowledge of structure of Ig subclasses, and the knowledge provided in the present invention, to select and to modify selected amino acids to prevent light chain interactions.

We have shown that the monovalent immunoglobulins or fragments thereof maintain a long in vivo half life when they comprise at least the CH2 and CH3 domains. In the present invention we now provide fusion proteins comprising a first molecule which is fused to a monovalent antibody or a fragment of a monovalent immunoglobulin comprising at least the CH2 and CH3 domains of the CH.

The present invention provides the following specific embodiments:
1) A fusion protein comprising at least first molecule and a monovalent immunoglobulin or a fragment of a monovalent immunoglobulin, wherein
   i) said first molecule and said monovalent immunoglobulin or fragment of a monovalent immunoglobulin are fused or linked either by peptide bonds or by other types of covalent bonding,
   ii) the monovalent immunoglobulin or the fragment of a monovalent immunoglobulin comprises at least the CH2 and CH3 regions of the CH,
   iii) wherein the monovalent immunoglobulin or fragment thereof as required by the Ig subtype, has been modified such that the CH3 region or other regions do not comprise any amino acid residues which are capable of participating in the formation of disulphide bonds or covalent or stable non-covalent inter-heavy chain bonds with other peptides comprising an identical amino acid sequence of the CH region of the immunoglobulin in the presence of polyclonal human Ig
   iv) if a C_{L} region or a fragment of a C_{L} region is not present, said monovalent immunoglobulin or fragment of a monovalent immunoglobulin does not comprise the CH1 region or has a CH1 region, where one or more amino acids in the hydrophobic patch have been changed to amino acids that are less hydrophobic, unless the CH1 region is of the camelidae type, and
   v) the hinge region of said monovalent immunoglobulin or fragment of the monovalent immunoglobulin is either deleted or mutated to remove any cysteine residues.
2) A fusion protein according to embodiment 1, wherein the first molecule is one of the following: a polypeptide such as a cytokine, a peptide mimetic, a small organic molecule.
3) A fusion protein according to embodiments 1 or 2, wherein the monovalent immunoglobulin or fragment thereof does not comprise a V_{H} or a V_{L} region or a fragment thereof.
4) A fusion protein according to any one of embodiments 1-3, wherein the hinge region of the monovalent immunoglobulin or fragment thereof has been deleted.
5) A fusion protein according to any one of embodiments 1 to 4, wherein the first molecule is a polypeptide, which is fused to the N-terminal of the monovalent immunoglobulin or fragment thereof.
6) A fusion protein according to any one of embodiments 1-2 or 4-5, wherein a C_{L} region or a fragment of a C_{L} region is present.
7) A fusion protein according to any one of the embodiments 1-2 or 4-5, wherein the monovalent immunoglobulin or fragment of the monovalent immunoglobulin consists of
   i) a CH1, CH2 and CH3 domains of the CH, wherein the CH1 and/or CH3 domains contain the modifications defined in herein above, or
   ii) a CH region, wherein the hinge region, CH1 and/or CH3 domains contain the modifications defined in (i) or (ii), or
   iii) a VH region and a CH region as defined in (i) or (ii), or
   iv) a VH region, a CH region as defined in (i) or (ii), and a CL region, or
   v) a VH, a CH region as defined in (i) or (ii), a VL region and a CL region
8) A fusion protein according to any one of the preceding embodiments, wherein the first molecule comprises two polypeptides fused to the monovalent immunoglobulin or fragment of the monovalent immunoglobulin, wherein the first polypeptide is fused or linked to the heavy chain of the monovalent immunoglobulin and the second polypeptide is fused or linked to the light chain.
9) A fusion protein according to embodiment 8, wherein the two polypeptides fused to the monovalent immunoglobulin are different.
10) A fusion protein according to any one of the preceding embodiments, wherein
   i) the monovalent immunoglobulin or fragment thereof comprises a variable region, and wherein the fusion protein comprises
   ii) a linker molecule comprising one part that is capable of being bound by a variable region, and second part that is capable of binding to the first molecule, and
11) A fusion protein according to any one of embodiments 1 to 9, wherein the first molecule is a polypeptide, such as a cytokine, which polypeptide is fused to the monovalent immunoglobulin or fragment thereof by peptide bonding, and wherein one or more amino acids have been inserted as spacers between the polypeptide and the monovalent immunoglobulin or fragment thereof.
12) A fusion protein according to any one of embodiments 1 to 9, wherein the first molecule is covalently linked to the monovalent immunoglobulin or fragment thereof by other covalent bonds than peptide bonding, and wherein a linker molecule is inserted between the two peptides.
13) A fusion protein according to any one of embodiments 1-12, wherein the Ig is IgG1, IgG2, IgG3, IgG4, IgA1, IgA2 or IgD.
14) A fusion protein according to any one of embodiments 1-13, wherein the Ig is human.
15) A fusion protein according to embodiment 14, wherein the human Ig comprises the amino acid C_{H}3 region as set forth in SEQ ID NO: 19, wherein the C_{H}3 region has been modified so that one or more of the following amino acid substitutions have been made: Arg (R) in position 238 has been replaced by Gln (Q); Asp (D) in position 239 has been replaced by Glu (E); Thr (T) in position 249 has been replaced by Ala (A); Leu (L) in position 251 has been replaced by Ala (A); Leu (L) in position 251 has been replaced by Val (V); Phe (F) in position 288 has been replaced by Ala (A); Phe (F) in position 288 has been replaced by Leu (L); Tyr (Y) in position 290 has been replaced by Ala (A); Lys (K) in position 292 has been replaced by Arg (R); Lys (K) in position 292 has been replaced by Ala (A); Gln (Q) in position 302 has been replaced by Glu (E); and Pro (P) in position 328 has been replaced by Leu (L).
16) A fusion protein according to embodiment 15, wherein Arg (R) in position 238 has been replaced by Gln (Q).
17) A fusion protein according to embodiment 15, wherein Arg (R) in position 238 has been replaced by Gln (Q), and Pro (P) in position 328 has been replaced by Leu (L).
18) A fusion protein according to embodiment 15, wherein all nine amino acids have been substituted.
19) A fusion protein according to embodiment 14, wherein the human Ig comprises the amino acid C_{H}3 region as set forth in SEQ ID NO: 20, wherein the C_{H}3 region has been modified so that one or more of the of the following amino acid substitutions have been made: Arg (R) in position 234 has been replaced by Gln (Q); Thr (T) in position 245 has been replaced by Ala (A); Leu (L) in position 247 has been replaced by Ala (A); Leu (L) in position 247 has been replaced by Val (V); Met (M) in position 276 has been replaced by Val (V); Phe (F) in position 284 has been replaced by Ala (A); Phe (F) in position 284 has been replaced by Leu (L); Tyr (Y) in position 286 has been replaced by Ala (A); Lys (K) in position 288 has been replaced by Arg (R); Lys (K) in position 288 has been replaced by Ala (A); Gln (Q) in position 298 has been replaced by Glu (E); and Pro (P) in position 324 has been replaced by Leu (L).
20) A fusion protein according to embodiment 19, wherein Arg (R) in position 234 has been replaced by Gln (Q).
21) A fusion protein according to embodiment 19, wherein Arg (R) in position 234 has been replaced by Gln (Q); and Pro (P) in position 324 has been replaced by Leu (L).
22) A fusion protein according to embodiment 19, wherein all nine amino acids have been substituted.
23) A fusion protein according to embodiment 14, wherein the Ig comprises the amino acid C_{H} region as set forth in SEQ ID NO: 21, wherein the C_{H}3 region has been modified so that one or more of the following amino acid substitutions have been made: Arg (R) in position 285 has been replaced by Gln (Q); Thr (T) in position 296 has been replaced by Ala (A); Leu (L) in position 298 has been replaced by Ala (A); Leu (L) in position 298 has been replaced by Val (V); Ser (S) in position 314 has been replaced by Asn (N); Asn (N) in position 322 has been replaced by Lys (K); Met (M) in position 327 has been replaced by Val (V); Phe (F) in position 335 has been replaced by Ala (A); Phe (F) in position 335 has been replaced by Leu (L); Tyr (Y) in position 337 has been replaced by Ala (A); Lys (K) in position 339 has been replaced by Arg (R); Lys (K) in position 339 has been replaced by Ala (A); Gln (Q) in position 349 has been replaced by Glu (E); Ile (I) in position 352 has been replaced by Val (V); Arg (R) in position 365 has been replaced by His (H); Phe (F) in position 366 has been replaced by Tyr (Y); and Pro (P) in position 375 has been replaced by Leu (L).
24) A fusion protein according to embodiment 23, wherein Arg (R) in position 285 has been replaced by Gln (Q).
25) A fusion protein according to embodiment 23, wherein Arg (R) in position 285 has been replaced by Gln (Q); and Pro (P) in position 375 has been replaced by Leu (L).
26) A fusion protein according to embodiment 23, wherein all 14 amino acids have been substituted.
27) A fusion protein according to any one of embodiments 1-14, wherein the monovalent immunoglobulin or fragment thereof comprises the human IgG4 CH2 and CH3 sequences as set forth in SEQ ID NO: 16.
28) A fusion protein according to any one of embodiments 1-14, wherein the monovalent immunoglobulin or fragment thereof consists of the human IgG4 CH2 and CH3 sequences as set forth in SEQ ID NO: 16.
29) A fusion protein according to any one of embodiments 1-14, wherein the monovalent immunoglobulin comprises the CH2 and CH3 sequence as set forth in SEQ ID NO: 16, but wherein the C_{H}3 region has been modified so that one or more of the following amino acid substitutions have been made: Thr (T) in position 234 has been replaced by Ala (A); Leu (L) in position 236 has been replaced by Ala (A); Leu (L) in position 236 has been replaced by Val (V); Phe (F) in position 273 has been replaced by Ala (A); Phe (F) in position 273 has been replaced by Leu (L); Tyr (Y) in position 275 has been replaced by Ala (A); Arg (R) in position 277 has been replaced by Ala (A);
30) A fusion protein according to embodiment 29, wherein all 5 amino acids have been substituted.
31) A fusion protein according to any of embodiments 1-30, wherein the amino acid sequence corresponding to the hinge region of the CH region of said immunoglobulin has been deleted.
32) A fusion protein according to any one of embodiments 1-14 and 27-30, wherein the amino acid sequence of a heavy chain of a human IgG4 has been modified such that said heavy chain comprises a C_{H} region, wherein the amino acid residues corresponding to amino acid residues 106 and 109 of the sequence of SEQ ID No: 14 have been substituted with amino acid residues different from cysteine.
33) A fusion protein according to any one of the preceding embodiments 1-30, wherein the C_{H} region of the monovalent antibody or fragment thereof, has been modified such that the region corresponding to the hinge region of the C_{H} region does not comprise any cysteine residues.
34) A fusion protein according to any one of the preceding embodiments 1-30, wherein the C_{H} region has been modified such that at least all cysteine residues of the hinge region have been deleted and/or substituted with other amino acid residues.
35) A fusion protein according to embodiment 34, wherein the C_{H} region has been modified such that the cysteine residues of the hinge region have been substituted with amino acid residues that have an uncharged polar side chain, or a nonpolar side chain.
36) A fusion protein according to embodiment 35, wherein the amino acids with uncharged polar side chains are independently selected from asparagine, glutamine, serine, threonine, tyrosine, and tryptophan, and the amino acid with the nonpolar side chain are independently selected from alanine, valine, leucine, isoleucine, proline, phenylalanine, and methionine.
37) A fusion protein according to any one of the preceding embodiments 1-14 or 27-30, wherein the monovalent immunoglobulin or fragment thereof is a human IgG4, wherein one of the amino acid residues corresponding to amino acid residues 106 and 109 of the sequence of SEQ ID No: 14 has been substituted with an amino acid residue different from cysteine, and the other of the amino acid residues corresponding to amino acid residues 106 and 109 of the sequence of SEQ ID No: 14 has been deleted.
38) A fusion protein according to any one of the preceding embodiments 1-14 or 27-30, wherein the monovalent immunoglobulin or fragment thereof is a human IgG4, wherein the amino acid residue corresponding to amino acid residue 106 has been substituted with an amino acid residue different from cysteine, and the amino acid residue corresponding to amino acid residue 109 has been deleted.
39) A fusion protein according to any one of the preceding embodiments 1-14 or 27-30, wherein the monovalent immunoglobulin or fragment thereof is a human IgG4, wherein the amino acid residue corresponding to amino acid residue 106 has been deleted, and the amino acid residue corresponding to amino acid residue 109 has been substituted with an amino acid residue different from cysteine.
40) A fusion protein according to any one of the preceding embodiments 1-14 or 27-30, wherein the monovalent immunoglobulin or fragment thereof is a human IgG4, which is a human IgG4, wherein at least the amino acid residues corresponding to amino acid residues 106 to 109 of the C_{H} sequence of SEQ ID No: 14 has been deleted.
41) A fusion protein according to any one of the preceding embodiments 1-14 or 27-30, wherein the monovalent immunoglobulin or fragment thereof is a human IgG4, wherein at least the amino acid residues corresponding to amino acid residues 99 to 110 of the sequence of SEQ ID No: 14 has been deleted.
42) A fusion protein according to any one of the preceding embodiments 1-14 or 27-30, wherein the monovalent immunoglobulin or fragment thereof is a human IgG4, wherein the C_{H} region comprises the amino acid sequence of SEQ ID No: 16.
43) A fusion protein according to any one of the preceding embodiments 1-14 or 27-30, wherein the monovalent immunoglobulin or fragment thereof is a human IgG4, wherein the C_{H} region has been modified such that the entire hinge region has been deleted.
44) A fusion protein according to any one of embodiments 1-43, wherein said fusion protein has a plasma concentration above 10 mg/ml for more than 7 days when administered in vivo to a human being or to a SCID mouse at a dose of 4 mg/kg.
45) A fusion protein according to any of embodiments 1 to 44, wherein said fusion protein has a half-life of at least 5 days when administered in vivo.
46) A fusion protein according to any one of embodiments 1 to 45, wherein said fusion protein has a half-life of at least 5 and up to 21 days when administered in vivo to a human being or a SCID mouse.
47) A fusion protein according to any one of embodiments 1 to 46, wherein said fusion or linked protein is capable of binding to FcRn.
48) A fusion protein according to any one of embodiments 1-47, wherein the molecule linked to the monovalent immunoglobulin or fragment thereof is a small organic molecule.
49) A fusion protein according to any one of embodiments 1 to 47, wherein the first molecule is selected from erythropoietin, thrombopoietin, interferon-alpha (2a and 2b), -beta (1b), -gamma, TNFR I (CD120a), TNFR II (CD120b), IL-1R type 1 (CD121a), IL-1R type 2 (CD121b), IL-2, IL2R (CD25), IL-2R-beta (CD123), IL-3, IL-4, IL-3R (CD123), IL-4R (CD124), IL-5R (CD125), IL-6R-alpha (CD126), -beta (CD130), IL-10, IL-11, IL-15BP, IL-15R, IL-20, IL-21, TCR variable chain, RANK, RANK-L, CTLA4, CXCR4R, CCR5R, TGF-beta1, -beta2, -beta3, G-CSF, GM-CSF, MIF-R (CD74), M-CSF-R (CD115), GM-CSFR (CD116), soluble FcgammaRI, sFcgammaRIIa, sFcgammaRIIb, sFcgammaRIIIa, sFcgammaRIIIb, sFcRn, sFcepsilonRI, sFcepsilonRIIa, sFcepsilonRIIb, sFcalphaI, Factor VII, Factor VIII, Factor IX, VEGF, VEGFxxxb, soluble Siglec-1, sSiglec-2, sSiglec-3, sSiglec-4, sSiglec-5, sSiglec-6, sSiglec-7, sSiglec-8, sSiglec-9, sSiglec-10, sSiglec-11, sSiglec-12, sSiglec-14, sSiglec-15.
50) A fusion protein according to any one of embodiments 1 to 49, wherein the first molecule is selected from anti-psychotic drugs, anti-depressant drugs, anti-Parkinson drugs, anti-seizure agents, neuromuscular blocking drugs, anti-epileptic drugs, adrenocorticosteroids, insulin, proteins or enzymes involved in regulation of insulin, incretins (GIP and GLP-1) or drugs mimicking incretin action such as Exenatide and sitagliptin, thyroid hormones, growth hormone, ACTH, oestrogen, testosterone, anti-diuretic hormone, diuretics, all kinds of blood products such as heparin and EPO, beta-blocking agents, cytotoxic agents, anti-viral drugs, anti-bacterial agents, anti-fungal agents, anti-parasitic drugs, anti-coagulation drugs, anti-inflammatory drugs, anti-asthma drugs, and anti-COPD drugs.
51) A fusion protein according to embodiment 48, wherein the first molecule is selected from sildenafil citrate (Viagra), opiates, morphine, vitamins (such as vitamin C for conservation), hormones involved in pregnancy such as LH and FSH, hormones involved in sex changes, anti-conceptives, and antibodies
52) A fusion protein according to any one of the preceding embodiments 1-51, wherein the first molecule is selected from single-chain Fv, dAb or domain antibody, nanobody, VHH, diabody, V-NAR, ScFab, CTL-4, tendamistat, 10^{th} fibronectin type 3 domain, neocarzinostatin, CBM4-2, Lipocalins, T-cell receptor, Protein A domain (protein Z), Im9, Designed AR proteins, Zinc finger, pVIII, Avian pancreatic polypeptide, GCN4, WW domain, Src homology domain 3 (SH3), Src homology domain 2, PDZ domains, TEM-1 □-lactamase, GFP, Thioredoxin, Staphylococcal nuclease, PHD-finger, CL-2, BPTI, APPI, HPSTI, Ecotin, LACI-D1, LDTI, MTI-II, Scorpion toxins, Insect defensin A peptide, EETI-II, Min-23, CBD, PBP, cytochrome b₅₆₂, Ldl receptor domain A, □-chrystallin, ubiquitin, transferring, C-type lectin-like domain (the scaffolds are described in Table 1 of Binz et al. (2005) Nature Biotechnology, vol. 23, no. 10, page 1257-1268).

In one specific embodiment a fusion protein according to any one of embodiments 1 to 49, is provided, wherein the first molecule is IL-7 (interleukin 7).

The present invention also provides pharmaceutical compositions comprising the fusion proteins according to the invention.

The present invention also provides pharmaceutical compositions further comprising one or more pharmaceutically acceptable excipients, diluents or carriers.

The present invention also provides pharmaceutical compositions comprising fusion proteins and, wherein the composition further comprises one or more further therapeutic agents.

The present invention also provides fusion proteins, for use as a medicament.

The present invention also provides fusion proteins, for use in the treatment of cancer, psychosis, depression, Parkinsons disease, seizure, neuromuscular diseases, epilepsia, diabetes, bacterial or viral infections, fungus infections, coagulation disorders, asthma, COPD.

The present invention also provides fusion proteins, for use in the treatment of an inflammatory condition.

The present invention provides fusion proteins, for use in the treatment of an auto(immune) disorder.

The present invention also provides fusion proteins, for use in the treatment of a disorder involving undesired angiogenesis.

The present invention also provides the use of a fusion protein as a medicament.

The present invention also provides the use of a fusion protein in the preparation of a medicament for the treatment of a disease as defined above, wherein the treatment comprises administering one or more further therapeutic agents.

The present invention also provides the use of a fusion protein in a method of treating a disease or disorder as defined above, wherein said method comprises administering to a subject in need of such treatment a therapeutically effective amount of a fusion protein or a pharmaceutical composition comprising a fusion protein.

The present invention also provides the use of a fusion protein in a method of treatment, wherein the treatment comprises administering one or more further therapeutic agents.

The present invention also provides the use of a fusion protein as a diagnostic agent.

The present invention also provides a nucleic acid construct, encoding the fusion protein of the invention, wherein the fusion protein comprise two polypeptides fused by peptide bonds, optionally separated by a peptide linker.

The present invention also provides a nucleic acid construct, encoding the fusion protein, wherein said nucleic acid construct is an expression vector.

The present invention also provides a nucleic acid construct encoding the fusion protein of the invention, for use in gene therapy.

The present invention also provides a pharmaceutical composition which comprises the nucleic acid construct for gene therapy.

The present invention also provides a method for preparing a fusion protein according to the invention, wherein the first molecule is a cytokine or other polypeptide, said method comprising the following steps:
i) providing an expression system comprising a nucleotide encoding the polypeptide,
ii) providing an expression system comprising a nucleotide encoding the monovalent immunoglobulin or a fragment thereof,
iii) expressing said cytokine or other peptide, and said monovalent immunoglobulin
iv) recovering and purifying said expressed proteins
v) combining the purified proteins by covalent binding

The present invention also provides a method of preparing a fusion protein according to the invention, said method comprising:
i) providing a nucleic acid construct encoding a fusion protein of said cytokine or other peptide and a monovalent immunoglobulin or a fragment of a monovalent immunoglobulin according to the invention,
ii) wherein said nucleotide sequence encoding said monovalent immunoglobulin or fragment thereof are operably linked together.
iii) providing a cell expression system for producing said fusion protein;
iv) producing said fusion protein by expressing said nucleic acid construct in cells of the cell expression system of iii)
v) recovering said expressed fusion protein from the media or cells

The present invention also provides a host cell comprising a nucleic acid according to the invention, as described above.

The present invention also provides a host cell, which host cell is a prokaryotic cell, such as an E. coli cell.

The present invention also provides a host cell, which host cell is a eukaryotic cell, such as a mammalian cell, insect, plant or a fungal cell.

The present invention also provides a non human transgenic animal comprising a nucleic acid construct according to the invention, as described above.

According to the invention, the amino acid sequence of the V_{L} region of the monovalent antibody does not contribute to the molecular properties of said antibody molecule which are of interest of the invention, in particular the inability of the monovalent antibody to form heterotetramers (“normal” antibodies), and therefore the invention is not limited to any particular amino acid sequences of the V_{L} region, if a V_{L} region is present. The amino acid sequence of the V_{L} region may be derived from the amino acid sequence of any antigen specific antibody generated in any of the many ways known to a person skilled in the art. According to the invention, the amino acid sequence of the V_{H} region of the monovalent antibody does not contribute to the molecular properties of said antibody molecule which are of interest of the invention, in particular the inability of the monovalent antibody to form heterotetramers (“normal” antibodies), and therefore the invention is not limited to any particular amino acid sequences of the V_{H} region, if a V_{H} region is present. The amino acid sequence of the V_{H} region may be derived from the amino acid sequence of any antigen specific antibody generated in any of the many ways known to a person skilled in the art.

In one embodiment, the monovalent antibody of the invention does not bind to the synthetic antigen (Tyr, Glu), Ala, Lys (Pincus et al. 1985, Molecular Immunolog, vol 22, 4; pp. 455-461)

In another embodiment, the antibody of the invention is a human antibody.

In another embodiment, the antibody of the invention is based on a human antibody.

The invention provides an example of 1) a monovalent antibody comprising a V_{H} region comprising the amino acid sequence of the V_{H} region of HuMab-7D8 identified as SEQ ID No: 6 and the amino acid sequence encoding the hingeless C_{H} of IgG4 identified as SEQ ID No: 16, wherein said sequences are operably linked together, and 2) a monovalent antibody comprising a V_{H} region comprising the amino acid sequence of the V_{H} region of mouse anti-Betv-1 identified as SEQ ID No: 8 and the amino acid sequence encoding the hingeless C_{H} of IgG4 identified as SEQ ID No: 16, wherein said sequences are operably linked together. In one embodiment, the V_{H} and V_{L} region of an antibody molecule of the invention are derived from the same antigen specific antibody.

According to the invention, the sequence of the C_{L} region of the light chain of the antibody molecule may be derived from the sequence of C_{L} region of an immunoglobulin. In one embodiment, the C_{L} region is the constant region of the kappa light chain of human IgG. In one embodiment, the C_{L} region comprises the amino acid sequence of SEQ ID No: 2. In one embodiment, the C_{L} region is the constant region of the lambda light chain of human IgG. In one embodiment, the C_{L} region comprises the amino acid sequence of SEQ ID No: 4.

In one embodiment, the light chain and the heavy chain of the monovalent antibody of the invention are connected to each other via one or more disulphide bond. It is evident that for such disulphide bonds, neither of the binding partners in the disulphide bond is present in the region corresponding to the hinge region.

In one embodiment, the light chain and the heavy chain are connected to each other via an amide bond, for instance as it is seen for single chain Fv's.

The hinge region is a region of an antibody situated between the C_{H}1 and C_{H}2 regions of the constant domain of the heavy chain. The extent of the hinge region is determined by the separate exon, which encodes the hinge region. The hinge region is normally involved in participating in ensuring the correct assembly of the four peptide chains of an antibody into the traditional tetrameric form via the formation of disulphide bonds, or bridges, between one or more cysteine residues in the hinge region of one of the heavy chains and one or more cysteine residues in the hinge region of the other heavy chain. A modification of the hinge region so that none of the amino acid residues in the hinge region are capable of participating in the formation of disulphide bonds may thus for instance comprise the deletion and/or substitution of the cysteine residues present in the unmodified hinge region. A region corresponding to the hinge region should for the purpose of this specification be construed to mean the region between region C_{H}1 and C_{H}2 of a heavy chain of an antibody. In the context of the present invention, such a region may also comprise no amino acid residues at all, corresponding to a deletion of the hinge region, resulting in the C_{H}1 and C_{H}2 regions being connected to each other without any intervening amino acid residues. Such a region may also comprise only one or a few amino acid residues, which residues need not be the amino acid residues present in the N- or C-terminal of the original hinge region. Disulphide bonds is a well-known feature of certain proteins, for instance antibodies, where one cysteine residue form a disulphide bond with another cysteine residue on the same chain (intra-chain disulphide bonds) or other chains (inter-chain disulphide bonds) of the protein. There may be several such disulphide bonds within a given protein. For antibodies, the formation of disulphide bonds, both intra-chain and inter-chain, is an integral part of the correct assembly of the fully matured wildtype antibody, and the disulphide-bonds are normally at least partly responsible for the highly ordered and regular appearance of antibodies as well as for the stability of the antibody. In the monovalent antibodies of the invention, none of the amino acids of the hinge region are capable of participating in the formation of such disulphide bonds.

The modification of the amino acid sequence of the hinge region may be performed on DNA level by use of recombinant techniques enabling the deletion and/or substitution of amino acids in the expressed protein by the deletion and/or substitution of nucleic acids as it is well known in the art and as it is described elsewhere herein and exemplified in the Examples.

The modification may also be performed on an antibody expressed from a non-modified nucleic acid by for instance derivatizing the amino acid residues in the hinge region, which amino acid residues are capable of forming disulphide bonds. Such derivatization of the cysteine residues blocking them from forming disulphide bonds with other cysteine residues may be performed as it is known in the art.

The modification may also be performed by prepared the chains of the antibodies synthetically by using amino acid residues other than cysteine, for instance naturally occurring amino acids or non-naturally occurring amino acids, such as for instance derivatized cysteines, instead of the cysteine residues.

A monovalent antibody of the present invention may also be an IgG4 variant. Such a variant antibody is an antibody that differs from a IgG4 antibody by one or more suitable amino acid residue alterations, that is substitutions, deletions, insertions, or terminal sequence additions, for instance in the constant domain, and/or the variable regions (or any one or more CDRs thereof) in a single variant antibody. Typically, amino acid sequence alterations, such as conservative substitution variations, desirably do not substantially change the structural characteristics of the parent sequence (e.g., a replacement amino acid should not tend to disrupt secondary structure that characterizes the function of the parent sequence), but which may be associated with advantageous properties, such as changing the functional or pharmacokinetic properties of the antibodies, for example increasing the half-life, altering the immunogenicity, providing a site for covalent or non-covalent binding to another molecule, reducing susceptibility to proteolysis, reducing susceptibility to oxidation, or altering the glycosylation pattern. Examples of variants include variants which have a modification of the CH3 region, such as a substitution or deletion at any one or more of the positions 225, 234, 236, 238, 273 or 275 of SEQ ID NO: 16 or the corresponding residues in non-IgG4 isotypes. Modifications at these positions may e.g. further reduce intermolecular interactions between hinge-modified antibodies of the invention. Other examples include variants which have a modification of the constant region, such as a substitution or deletion, at any one or more of the positions 118, 120, 122, 124, 175, 248, 296, 302 of SEQ ID NO: 16 or the corresponding residues in non-IgG4 isotypes. Modifications at these positions may e.g. increase the half-life of hinge-modified antibodies of the invention.

In one embodiment, the amino acid sequence of the heavy chain has been modified such that the region corresponding to the hinge region does not comprise any cysteine residues. In one embodiment, the amino acid sequence of the heavy chain has been modified such that at least one of the amino acid residues of the region corresponding to the hinge region, including any cysteine residues, have been deleted and/or substituted with other amino acid residues. The hinge region of antibodies of the invention may thus be modified in other positions than the positions, in which any cysteine residues are normally present, as also described above for variant IgG4 antibodies of the invention. Such modifications may be performed as described above or by any other means known in the art.

In the context of the present invention, the cysteine residues of the region corresponding to the hinge region may be substituted by any naturally occurring or non-naturally occurring, and/or non-L amino acid residues other than cysteine or with derivatives of such amino acid residues including derivatives of cysteine residues, which derivatized cysteine residues are incapable of participating in the formation of disulphide bonds.

If a hinge region is present in the fusion proteins of the present invention, the following embodiment in non limiting example would apply:

In one embodiment, the amino acid sequence of the heavy chain has been modified such that the heavy chain comprises a C_{H} region, wherein the amino acids corresponding to amino acids 106 and 109 of the sequence of SEQ ID No: 14 has been deleted. SEQ ID No: 14 shows an amino acid sequence of a wildtype C_{H} region of human IgG4 and positions 106 and 109 are the positions of the two cysteine residues.

In one embodiment, the amino acid sequence of the heavy chain has been modified such that the heavy chain comprises a C_{H} region, wherein at least the amino acid residues corresponding to amino acid residues 106 to 109 of the sequence of SEQ ID No: 14 has been deleted.

In one embodiment, the amino acid sequence of the heavy chain has been modified such that the heavy chain comprises a C_{H} region, wherein at least the amino acid residues corresponding to amino acid residues 99 to 110 of the sequence of SEQ ID No: 14 has been deleted.

In one embodiment, the heavy chain comprises the amino acid sequence of SEQ ID No: 16. SEQ ID No: 16 is the amino acid sequence of the C_{H} region of a human IgG4 generated by expression of the nucleic acid comprising the sequence of SEQ ID No: 15, which is a nucleic acid sequence encoding the C_{H} region of human IgG4 (SEQ ID No: 13) carrying substitution mutations in positions 714 and 722. These substitutions in the splice donor site of the nucleic acid sequence has the effect that the splicing involving the exon encoding the hinge region will not be performed correctly resulting in a heavy chain without the amino acids residues encoded by the exon.

In one embodiment, the entire hinge region of the C_{H} region has been deleted. This is the case where no amino acids encoded by the exon encoding the hinge region of the C_{H} region is present in the heavy chain. For the IgG4 shown in SEQ ID No: 14, this will correspond to a C_{H} region having the amino acid sequence of SEQ ID No: 16.

In one embodiment, the amino acid sequence of the heavy chain has been modified such that the heavy chain comprises a C_{H} region, wherein the amino acid residues corresponding to amino acid residues 106 and 109 of the sequence of SEQ ID No: 14 has been substituted with amino acid residues different from cysteine.

In one embodiment, the amino acid sequence of the heavy chain has been modified such that the heavy chain comprises a C_{H} region, wherein one of the amino acid residues corresponding to amino acid residues 106 and 109 of the sequence of SEQ ID No: 14 has been substituted with an amino acid residue different from cysteine and the other of the amino acid residues corresponding to amino acid residues 106 and 109 of the sequence of SEQ ID No: 14 has been deleted. In a further embodiment, it is the amino acid residue corresponding to amino acid residues 106, which has been substituted with an amino acid residue different from cysteine, and the amino acid residue corresponding to amino acid residues 109, which has been deleted. In another further embodiment, it is the amino acid residue corresponding to amino acid residues 106, which has been deleted, and the amino acid residue corresponding to amino acid residues 109, which has been substituted with an amino acid residue different from cysteine.

### Further Embodiments

In one embodiment, fusion protein of the invention has a plasma concentration above 10 μg/ml for more than 7 days when administered in vivo at a dose of 4 mg per kg, as measured in an pharmacokinetic study in SCID mice (for instance as shown in example 32). The clearance rate of a fusion protein of the invention may be measured by use of pharmacokinetic methods as it is known in the art. The fusion protein may for instance be injected intravenously (other routes such as i.p. or i.m. may also be used) in a human or animal after which blood samples are drawn by venipuncture at several time points, for instance 1 hour, 4 hours, 24 hours, 3 days, 7 days, 14 days, 21 days and 28 days after initial injection). The concentration of fusion protein in the serum is determined by an appropriate assay such as ELISA. Pharmacokinetic analysis is performed as known in the art and described in example 32. Monovalent antibodies of the invention may have a plasma residence time, which is as much as 100 times longer than the plasma residence time of for instance Fab fragments which are frequently used as monovalent antibodies. In one embodiment, a fusion protein of the invention has a plasma clearance, which is more than 10 times slower than the plasma clearance of a F(ab′)₂ fragment, which has a comparable molecular size. This may be an indication of the capability of the fusion proteins of the invention to bind to FcRn. FcRn is a major histocompatibility complex class I-related receptor and plays a role in the passive delivery of immunoglobulin (Ig)Gs from mother to young and in the regulation of serum IgG levels by protecting IgG from intracellular degradation (Ghetie V et al., Annu Rev Immunol. 18, 739-66 (2000)).

In one embodiment, a fusion protein of the invention has a half-life of at least 5 days when administered in vivo. The half-life of a fusion protein of the invention may be measured by any method known in the art, for instance as described above.

In one embodiment, a fusion protein of the invention has a half-life of at least 5 days and up to 14 days, when administered in vivo.

In one embodiment, a fusion protein of the invention has a half-life of at least 5 days and up to 21 days, when administered in vivo.

In one embodiment, a fusion protein of the invention is capable of binding to FcRn. Such binding may be determined by use of methods for determining binding as it is known in the art, for instance by use of ELISA assays. The binding of a fusion protein of the invention to FcRn may for instance be compared to the binding of a F(ab′)₂ fragment, which F(ab′)₂ fragment has a V_{H} region and a V_{L} region, which are identical to the V_{H} region and the V_{L} region (if present) of the monovalent immunoglobulin that is part of the fusion protein of the invention, to FcRn in the same assay. In one embodiment, the binding of an a fusion protein of the invention to FcRn is more than 10 times stronger than the binding of the F(ab′)₂ fragment to FcRn.

Fusion proteins, such as the fusion proteins of the invention, may often be useful in the treatment of diseases or disorders, where a long in vivo half life of first molecule of the fusion protein is desirable, and where effector function from the antibody is undesirable, since the fusion proteins of the inventions due to their monovalent nature do not exhibit effector functions such as ADCC or CDC.

In one embodiment, a fusion protein of the invention is incapable of effector binding. The expression “incapable of effector binding” or “inability of effector binding” in the present context means that a fusion protein of the invention is incapable of binding to the C1q component of the first component of complement (C1) and therefore is unable of activating the classical pathway of complement mediated cytotoxicity. In addition, the fusion proteins of the invention are unable to interact with Fc receptors and may therefore be unable to trigger Fc receptor-mediated effector functions such as phagocytosis, cell activation, induction of cytokine release

In one embodiment, a fusion protein of the invention is produced by use of recombinant DNA technologies. Antibodies may be produced using recombinant eukaryotic host cells, such as chinese hamster ovary (CHO) cells, NS/0 cells, HEK293 cells, insect cells, plant cells, or fungi, including yeast cells. Both stable as well as transient systems may be used for this purpose. Transfection may be done using plasmid expression vectors by a number of established methods, such as electroporation, lipofection or nucleofection. Alternatively, infection may be used to express proteins encoded by recombinant viruses such as adeno, vaccinia or baculoviruses. Another method may be to use transgenic animals for production of antibodies.

A DNA sequence encoding the fusion protein or the different polypeptides of the fusion protein may be prepared synthetically by established standard methods, for instance the phosphoamidine method described by Beaucage et al., Tetrahedron Lett. 22, 1859-1869 (1981), or the method described by Matthes et al., EMBO J. 3, 801-805 (1984). According to the phosphoamidine method, oligonucleotides are synthesised, for instance in an automatic DNA synthesiser, purified, annealed, ligated and cloned in suitable vectors.

A DNA sequence encoding the may also be of genomic or cDNA origin, for instance obtained by preparing a genomic or cDNA library and screening for DNA sequences coding for all or part of the antibody by hybridisation using synthetic oligonucleotide probes in accordance with standard techniques (cf. Sambrook et al., Molecular Cloning: A Laboratory Manual, 2nd Ed., Cold Spring Harbor, 1989). The DNA sequence may also be prepared by polymerase chain reaction using specific primers, for instance as described in U.S. Pat. No. 4,683,202 or Saiki et al. Science 239, 487-491 (1988).

The DNA sequence may then be inserted into a recombinant expression vector, which may be any vector, which may conveniently be subjected to recombinant DNA procedures. The choice of vector will often depend on the host cell into which it is to be introduced. Thus, the vector may be an autonomously replicating vector, i.e. a vector that exists as an extrachromosomal entity, the replication of which is independent of chromosomal replication, for instance a plasmid. Alternatively, the vector may be one which, when introduced into a host cell, is integrated into the host cell genome and replicated together with the chromosome(s) into which it has been integrated.

In the vector, a DNA sequence encoding the polypeptides should be operably connected to a suitable promoter sequence. The promoter may be any DNA sequence, which shows transcriptional activity in the host cell of choice and may be derived from genes encoding proteins either homologous or heterologous to the host cell. Examples of suitable promoters for directing the transcription of the coding DNA sequence in mammalian cells are the CMV promoter, the SV40 promoter, the MT-1 (metallothionein gene) promoter or the adenovirus 2 major late promoter. Other suitable promoters are known in the art. A suitable promoter for use in insect cells is for instance the polyhedrin promoter. Suitable promoters for use in yeast host cells include promoters from yeast glycolytic genes or alcohol dehydrogenase genes, or the TPI1 or ADH2-4c promoters. Suitable promoters for use in filamentous fungus host cells are, for instance, the ADH3 promoter or the tpiA promoter.

The coding DNA sequence may also be operably connected to a suitable terminator, such as the human growth hormone terminator or (for fungal hosts) the TPI1 or ADH3 terminators. Other suitable terminators are known in the art. The vector may further comprise elements such as polyadenylation signals (for instance from SV40 or the adenovirus 5 Elb region), transcriptional enhancer sequences (for instance the SV40 enhancer) and translational enhancer sequences (for instance the ones encoding adenovirus VA RNAs). Other such signals and enhancers are known in the art.

The recombinant expression vector may further comprise a DNA sequence enabling the vector to replicate in the host cell in question. An example of such a sequence (when the host cell is a mammalian cell) is the SV40 origin of replication. Other origins of replications are known in the art. The vector may also comprise a selectable marker, for instance a gene the product of which complements a defect in the host cell, such as the gene coding for dihydrofolate reductase (DHFR), glutamine synthetase (GS) or one which confers resistance to a drug, for instance neomycin, hydromycin or methotrexate. Other selectable markers are known in the art.

The procedures used to ligate the DNA sequences coding the peptides or full-length proteins, the promoter and the terminator, respectively, and to insert them into suitable vectors containing the information necessary for replication, are well known to persons skilled in the art (cf., for instance, Sambrook et al., op. cit.).

To obtain recombinant monovalent antibodies of the invention, the DNA sequences encoding different parts of the polypeptide chain(s) of the antibody may be individually expressed in a host cell, or may be fused, giving a DNA construct encoding the fusion polypeptide, such as a polypeptide comprising both light and heavy chains, inserted into a recombinant expression vector, and expressed in host cells.

The host cell into which the expression vector may be introduced, may be any cell which is capable of expression of full-length proteins, and may for instance be a eukaryotic cell, such as invertebrate (insect) cells or vertebrate cells, for instance Xenopus laevis oocytes or mammalian cells, such as insect and mammalian cells. Examples of suitable mammalian cell lines are the HEK293 (ATCC CRL-1573), COS (ATCC CRL-1650), BHK (ATCC CRL-1632, ATCC CCL-10), NS/0 (ECACC 85110503) or CHO (ATCC CCL-61) cell lines. Other suitable cell lines are known in the art. In one embodiment, the expression system is a mammalian expression system, such as a mammalian cell expression system comprising various clonal variations of HEK293 cells. Also plant cell, bacterial and yeast expression systems may be utilized, especially if a non glycosylated form of a polypeptide is to be expressed.

Methods of transfecting mammalian cells and expressing DNA sequences introduced in the cells are described in for instance Kaufman et al., J. Mol. Biol. 159, 601-621 (1982); Southern et al., J. Mol. Appl. Genet. 1, 327-341 (1982); Loyter et al., Proc. Natl. Acad. Sci. USA 79, 422-426 (1982); Wigler et al., Cell 14, 725 (1978); Corsaro et al., Somatic Cell Genetics 7, 603 (1981); Graham et al., Virol. 52, 456 (1973); and Neumann et al., EMBO J. 1, 841-845 (1982). To obtain a fusion protein of the invention, host cells of the expression system may in one embodiment be cotransfected with two expression vectors simultaneously, wherein first of said two expression vectors comprises a DNA sequence encoding the immunoglobulin part of the fusion protein, and the second of said two expression vectors comprises a DNA sequence encoding the polypeptide of the first molecule of the fusion protein. The two sequences may also be present on the same expression vector, or they may be fused giving a DNA construct encoding the fusion polypeptide, such as a polypeptide comprising both the first molecule and the immunoglobulin operationally linked, optionally with a polypeptide spacer inserted between the two polypeptides of the fusion protein. In examples where polypeptides of the fusion protein are fused by peptide bonding, the polypeptide of the first molecule of the fusion protein is positioned at the N-terminal of the monovalent immunoglobulin or fragment of the monovalent immunoglobulin.

In one embodiment, fungal cells (including yeast cells) may be used as host cells. Examples of suitable yeast cells include cells of Saccharomyces spp. or Schizosaccharomyces spp., in particular strains of Saccharomyces cerevisiae. Examples of other fungal cells are cells of filamentous fungi, for instance Aspergillus spp. or Neurospora spp., in particular strains of Aspergillus oryzae or Aspergillus niger. The use of Aspergillus spp. for the expression of proteins is described in, for instance EP 238 023.

The medium used to culture the cells may be any conventional medium suitable for growing mammalian cells, such as a serum-containing or serum-free medium containing appropriate supplements, or a suitable medium for growing insect, yeast or fungal cells. Suitable media are available from commercial suppliers or may be prepared according to published recipes (for instance in catalogues of the American Type Culture Collection).

The recombinantly produced monovalent antibody may then be recovered from the culture medium by conventional procedures including separating the host cells from the medium by centrifugation or filtration, precipitating the proteinaceous components of the supernatant or filtrate by means of a salt, for instance ammonium sulphate, purification by a variety of chromatographic procedures, for instance HPLC, ion exchange chromatography, affinity chromatography, Protein A chromatography, Protein G chromatography, or the like.

The present invention also relates to a method of preparing a monovalent antibody of the invention, wherein said method comprises the steps of:
(a) culturing a host cell comprising a nucleic acid encoding said monovalent antibody; and
(b) recovering the monovalent antibody from the host cell culture.

In one embodiment, said host cell is a prokaryotic host cell. In one embodiment, the host cell is an E. coli cell. In one embodiment, the E. coli cells are of a strain deficient in endogenous protease activities.

In one embodiment, said host cell is a eukaryotic cell. In one embodiment, the host cell is a HEK-293F cell. In another embodiment, the host cell is a CHO cell.

In one embodiment, the monovalent antibody is recovered from culture medium. In another embodiment, the monovalent antibody is recovered from cell lysate.

The antibodies of the present invention has the advantage of having a long halflife in vivo, leading to a longer therapeutic window, as compared to e.g. a FAB fragment of the same antibody which has a considerably shorter halflife in vivo.

Further, due to the long halflife and small size, the fusion proteins of the invention potentially will have a better distribution in vivo, than fusion proteins comprising traditional tetrameric antibodies as stabilizers, in example by being able to penetrate solid tumors. And furthermore, the fusion proteins of the invention have the advantage for some uses that they do not exhibit effector functions such as ADCC.

Fusion proteins of the present invention are monovalent, are stable under physiological conditions, are unable to activate complement, and are thus suitable for use in treating disorders and diseases, in which the use of i.e. a cytokine with a long half life or wherein the activation of complement is unnecessary or disadvantageous.

The expression “stable under physiological conditions” or “stability under physiological conditions” in the present context means that the fusion protein retains its major structural and functional characteristics unchanged and is present in a therapeutically significant concentration for more than one week after said molecule is administered to a subject in vivo at a dose of 1 to 10 mg per kg. A plasma concentration of 5 μg/ml is considered to be significant for most therapeutic antibodies, because the antibodies may show saturation of target binding at this level. A time interval of 7 days is considered in this context to be relatively long.

Both in immune-deficient and in immune-competent mice, the clearance of the hingeless variant is much slower than that of F(ab′)₂ fragments, which have a comparable molecular size. This indicates that the Fc-part has a favorable effect on the plasma residence time in and provides indication of a functional interaction with the neonatal Fc receptor (FcRn) which protects endocytosed IgG from intracellular degradation. The clearance rate of the hingeless variant was about 300 times lower than that of Fab fragments, indicating that it may be given at a 300 times lower dosing for obtaining equivalent sustained plasma concentrations.

The invention also relates to an immunoconjugate of the fusion protein of the invention. The present invention features in particular a monovalent antibody of the invention conjugated to a therapeutic moiety, such as a cytotoxin, a chemotherapeutic drug, an immunosuppressant or a radioisotope. Such conjugates are referred to herein as “immunoconjugates”. A cytotoxin or cytotoxic agent includes any agent that is detrimental to (for instance kills) cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof.

Suitable chemotherapeutic agents for forming immunoconjugates of the invention include, but are not limited to, antimetabolites (for instance methotrexate, 6-mercaptopurine, 6-thioguanine, cytarabine, fludarabin, 5-fluorouracil, decarbazine, hydroxyurea, azathiprin, gemcitabin and cladribin), alkylating agents (for instance mechlorethamine, thioepa, chlorambucil, melphalan, carmustine (BSNU) and lomustine (CCNU), cyclophosphamide, busulfan, dibromomannitol, streptozotocin, mitomycin C, and cis-dichlorodiamine platinum (II) (DDP) cisplatin), anthracyclines (for instance daunorubicin (formerly daunomycin) and doxorubicin), antibiotics (for instance dactinomycin (formerly actinomycin), bleomycin, mithramycin, and anthramycin (AMC)), and anti-mitotic agents (for instance vincristine, vinblastine, docetaxel, paclitaxel and vinorelbin).

Suitable radioisotopes are for instance iodine-131, yttrium-90 or indium-111. Further examples of therapeutic moieties may be a protein or polypeptide possessing a desired biological activity. Such proteins may include, for example, an enzymatically active toxin, or active fragment thereof, such as abrin, ricin A, pseudomonas exotoxin, or diphtheria toxin; a protein such as tumor necrosis factor or interferon-γ; or biological response modifiers such as, for example, lymphokines, interleukin-1 (IL-1), interleukin-2 (IL-2), interleukin-6 (IL-6), granulocyte macrophage colony stimulating factor (GM-CSF), granulocyte colony stimulating factor (G-CSF), or other growth factors.

In one embodiment, the therapeutic moiety is doxorubicin, cisplatin, bleomycin, carmustine, chlorambucil, cyclophosphamide or ricin A.

Techniques for conjugating such therapeutic moiety to antibodies are well known, see, for instance Amon et al., “Monoclonal Antibodies For Immunotargeting Of Drugs In Cancer Therapy”, Monoclonal Antibodies And Cancer Therapy, Reisfeld et al. (eds.), pp. 243-56 (Alan R. Liss, Inc. 1985); Hellstrom et al., “Antibodies For Drug Delivery”, Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, Monoclonal Antibodies 1984: Biological And Clinical Applications, Pinchera et al. (eds.), pp. 475-506 (1985); “Analysis, Results, And Future Prospective Of The Therapeutic Use Of Radiolabeled Antibody In Cancer Therapy”, Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982).

In one embodiment, the fusion protein of the invention are attached to a linker-chelator, for instance tiuxetan, which allows for the antibody to be conjugated to a radioisotope.

In one embodiment, the present invention provides a pharmaceutical composition comprising a fusion protein of the present invention. The pharmaceutical compositions may be formulated with pharmaceutically acceptable carriers or diluents as well as any other known adjuvants and excipients in accordance with conventional techniques such as those disclosed in Remington: The Science and Practice of Pharmacy, 19^{th} Edition, Gennaro, Ed., Mack Publishing Co., Easton, Pa., 1995.

The pharmaceutical composition may be administered by any suitable route and mode. As will be appreciated by the skilled artisan, the route and/or mode of administration will vary depending upon the desired results.

The pharmaceutical compositions of the present invention include those suitable for oral, nasal, topical (including buccal and sublingual), rectal, vaginal and/or parenteral administration.

Formulations of the present invention which are suitable for vaginal administration include pessaries, tampons, creams, gels, pastes, foams or spray formulations containing such carriers as are known in the art to be appropriate. Dosage forms for the topical or transdermal administration of compositions of this invention include powders, sprays, ointments, pastes, creams, lotions, gels, solutions, patches and inhalants.

In one embodiment, the pharmaceutical composition is suitable for parenteral administration.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

In one embodiment the pharmaceutical composition is administered by intravenous or subcutaneous injection or infusion.

In one embodiment, the fusion protein of the invention are administered in crystalline form by subcutaneous injection, cf. Yang et al. PNAS, 100(12), 6934-6939 (2003).

Regardless of the route of administration selected, the fusion proteins of the present invention, which may be used in the form of a pharmaceutically acceptable salt or in a suitable hydrated form, and/or the pharmaceutical compositions of the present invention, are formulated into pharmaceutically acceptable dosage forms by conventional methods known to those of skill in the art.

As used herein, “pharmaceutically acceptable carrier” includes any and all solvents, dispersion media, coatings, antibacterial and antifungal agents, isotonicity agents, antioxidants and absorption delaying agents, and the like that are physiologically compatible.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the fusion protein, use thereof in the pharmaceutical compositions of the invention is contemplated.

In one embodiment, the carrier is suitable for parenteral administration, for instance intravenous or subcutaneous injection or infusion.

Pharmaceutical compositions typically must be sterile and stable under the conditions of manufacture and storage. The composition may be formulated as a solution, micro-emulsion, liposome, or other ordered structure suitable to high drug concentration. Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity may be maintained, for example, by the use of coating materials, such as lecithin, by the maintenance of the required particle size in the case of dispersions, and by the use of surfactants.

The pharmaceutical compositions may also contain adjuvants such as presservatives, wetting agents, emulsifying agents and dispersing agents. Prevention of presence of microorganisms may be ensured both by sterilization procedures and by the inclusion of various antibacterial and antifungal agents, for example, paraben, chlorobutanol, phenol, sorbic acid, and the like. It may also be desirable to include isotonicity agents, such as sugars, polyalcohols such as mannitol, sorbitol, glycerol or sodium chloride in the compositions. Pharmaceutically-acceptable antioxidants may also be included, for example (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Prolonged absorption of the injectable compositions may be brought about by including agents that delays absorption, for example, monostearate salts and gelatin. Sterile injectable solutions may be prepared by incorporating the monovalent antibody in the required amount in an appropriate solvent with one or a combination of ingredients for instance as enumerated above, as required, followed by sterilization microfiltration. Generally, dispersions are prepared by incorporating the fusion protein into a sterile vehicle that contains a basic dispersion medium and the required other ingredients for instance from those enumerated above. In the case of sterile powders for the preparation of sterile injectable solutions, examples of methods for preparation are vacuum drying and freeze-drying (lyophilization) that yield a powder of the active ingredient plus any additional desired ingredient from a previously sterile-filtered solution thereof.

If appropriate, the fusion protein may be used in a suitable hydrated form or in the form of a pharmaceutically acceptable salt. A “pharmaceutically acceptable salt” refers to a salt that retains the desired biological activity of the parent compound and does not impart any undesired toxicological effects (see for instance Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like. Depending on the route of administration, the monovalent antibody may be coated in a material to protect the compound from the action of acids and other natural conditions that may inactivate the compound. For example, the compound may be administered to a subject in an appropriate carrier, for example, liposomes. Liposomes include water-in-oil-in-water CGF emulsions as well as conventional liposomes (Strejan et al., J. Neuroimmunol. 7, 27 (1984)).

The fusion protein may be prepared with carriers that will protect the fusion protein against rapid release, such as a controlled release formulation, including implants, transdermal patches, and microencapsulated delivery systems. Biodegradable, biocompatible polymers may be used, such as ethylene vinyl acetate, polyanhydrides, polyglycolic acid, collagen, polyorthoesters, and polylactic acid. Methods for the preparation of such formulations are generally known to those skilled in the art, see for instance Sustained and Controlled Release Drug Delivery Systems, J. R. Robinson, ed., Marcel Dekker, Inc., New York, 1978. The pharmaceutical compositions may be administered with medical devices known in the art. In one embodiment, a therapeutic composition of the invention may be administered with a needleless hypodermic injection device, such as the devices disclosed in U.S. Pat. No. 5,399,163; U.S. Pat. No. 5,383,851; U.S. Pat. No. 5,312,335; U.S. Pat. No. 5,064,413; U.S. Pat. No. 4,941,880; U.S. Pat. No. 4,790,824; or U.S. Pat. No. 4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Many other such implants, delivery systems, and modules are known to those skilled in the art. In one embodiment, the fusion proteins of the invention may be formulated to ensure proper distribution in vivo for instance by use of liposomes. For methods of manufacturing liposomes, see for instance U.S. Pat. No. 4,522,811; U.S. Pat. No. 5,374,548; and U.S. Pat. No. 5,399,331. The liposomes may comprise one or more moieties which are selectively transported into specific cells or organs, thus enhance targeted drug delivery (see, for instance V. V. Ranade, J. Clin. Pharmacol. 29, 685 (1989)). Exemplary targeting moieties include folate or biotin (see, for instance U.S. Pat. No. 5,416,016); mannosides (Umezawa et al., Biochem. Biophys. Res. Commun. 153, 1038 (1988)); other antibodies (Bloeman et al., FEBS Lett. 357, 140 (1995); Owais et al., Antimicrob. Agents Chemother. 39, 180 (1995)); surfactant protein A receptor (Briscoe et al., Am. J. Physiol. 1233, 134 (1995)), different species of which may comprise the formulations of the inventions, as well as components of the invented molecules; p120 (Schreier et al., J. Biol. Chem. 269, 9090 (1994)); see also Keinanen et al., FEBS Lett. 346, 123 (1994); Killion et al., Immunomethods 4, 273 (1994). The composition must be fluid to the extent that easy syringability exists. It must be stable under the conditions of manufacture and storage and must be preserved against the contaminating action of microorganisms such as bacteria and fungi.

In one embodiment, the fusion proteins of the invention may be formulated to prevent or reduce their transport across the placenta. This may be done by methods known in the art, for instance by PEGylation of the fusion proteins. Further references may be made to Cunningham-Rundles et al., J Immunol Methods. 152, 177-190 (1992); and to Landor et al., Ann. Allergy Asthma Immunol. 74, 279-283 (1995).

Dosage regimens are adjusted to provide the optimum desired response (for instance a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of fusion protein calculated to produce the desired therapeutic effect in association with the required pharmaceutical carrier. The specification for the dosage unit forms of the invention are dictated by and directly dependent on (a) the unique characteristics of the fusion protein and the particular therapeutic effect to be achieved, and (b) the limitations inherent in the art of compounding such a fusion protein for the treatment of sensitivity in individuals.

Actual dosage levels of the fusion protein in the pharmaceutical compositions of the present invention may be varied so as to obtain an amount of the active ingredient which is effective to achieve the desired therapeutic response for a particular patient, composition, and mode of administration. The selected dosage level will depend upon a variety of pharmacokinetic factors including the activity of the particular monovalent antibodies of the present invention employed, the route of administration, the time of administration, the rate of excretion of the particular fusion protein being employed, the duration of the treatment, other drugs, compounds and/or materials used in combination with the particular compositions employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved. In general, a suitable dose of a pharmaceutical composition of the invention will be that amount of the fusion protein which is the lowest dose effective to produce a therapeutic effect. Such an effective dose will generally depend upon the factors described above. As another example, the physician or veterinarian may start with a high loading dose followed by repeated administration of lower doses to rapidly build up a therapeutically effective dose and maintain it over longer periods of time.

A pharmaceutical composition of the invention may contain one or a combination of different fusion proteins of the invention. Thus, in a further embodiment, the pharmaceutical compositions include a combination of multiple (for instance two or more) fusion proteins of the invention which act by different mechanisms. The fusion proteins may also be thus combined with divalent antibodies or with other types of therapeutic drugs.

The present invention also relates to a nucleic acid construct encoding the amino acid sequence of the C_{H} region of the heavy chain of a monovalent immunoglobulin of the invention.

In one embodiment, the invention provides a nucleic acid construct comprising a nucleic acid sequence encoding the C_{H} region of an IgG4, wherein the nucleic acid sequence encoding the C_{H} region has been modified such that the region corresponding to the hinge region in said C_{H} region does not comprise any amino acid residues capable of participating in the formation of disulphide bonds with peptides comprising an amino acid sequence identical to the amino acid sequence of said C_{H} region, or a sequence complementary thereof.

A nucleic acid construct encoding the C_{H} region of a monovalent antibody of the invention may be derived from nucleic acids encoding the C_{H} region of IgG4. The nucleic acid construct encoding the full-length amino acid sequence of the C_{H} region of IgG4 may be prepared by any of the methods discussed herein, for instance in the Examples, or in other ways known in the art. The methods of manipulation with recombinant DNA sequences are well known in the art, and may for instance be done by using site-directed mutagenises, such as described in the present specification. However, site-directed mutagenesis is just one of non-limited examples of the technologies that may be applied.

The modification of the nucleic acid sequence encoding the C_{H} region may be performed as described above for the construction of the fusion proteins of the invention.

In one embodiment, the nucleic acid sequence encoding the C_{H} region has been modified such that the region corresponding to the hinge region does not comprise any cysteine residues.

In one embodiment, the nucleic acid sequence encoding the C_{H} region has been modified such that at least one of the amino acid residues of the region corresponding to the hinge region, including any cysteine residues, have been deleted and/or substituted with other amino acid residues.

In one embodiment, the nucleic acid sequence encoding the C_{H} region has been modified such that the amino acids corresponding to amino acids 106 and 109 of the sequence of SEQ ID No: 14 have been deleted.

In one embodiment, the nucleic acid sequence encoding the C_{H} region has been modified such that at least the amino acid residues corresponding to amino acid residues 106 to 109 of the sequence of SEQ ID No: 14 has been deleted.

In one embodiment, the nucleic acid sequence encoding the C_{H} region has been modified such that at least the amino acid residues corresponding to amino acid residues 99 to 110 of the sequence of SEQ ID No: 14 has been deleted.

In one embodiment, the nucleic acid sequence encoding the C_{H} region has been modified such that the entire hinge region has been deleted.

In one embodiment, mutation (substitution) of nucleotides corresponding to the splice donor site of the hinge region in the sequence encoding the C_{H} region of IgG4, identified herein as SEQ ID No: 13, leads to expression of a polypeptide comprising a hingeless C_{H} region of IgG4.

Accordingly, in one embodiment, the nucleic acid construct of the invention has been modified such that at least one nucleotide of the splice donor site of the nucleic acid sequence encoding the hinge region has been substituted with a nucleotide different than the nucleotide originally present in that position.

In one embodiment, the nucleotides corresponding to the nucleotides in position 714 and 722 of the sequence of SEQ ID No: 13 has been substituted with a nucleotide different than the nucleotide present at that position in SEQ ID No: 13.

In one embodiment, the nucleic acid sequence encoding the C_{H} region of a nucleic acid construct of the invention comprises a sequence of SEQ ID No: 13, wherein nucleotides 714 and 722 of the sequence of SEQ ID No: 13 has been substituted with a nucleotide different than the nucleotide present at that position in SEQ ID No: 13.

In one embodiment, the nucleic acid sequence encoding the C_{H} region of a nucleic acid construct of the invention comprises the nucleotide sequence of SEQ ID No: 15.

In one embodiment, the nucleic acid sequence encoding the C_{H} region of a nucleic acid construct of the invention has been modified such that the amino acid residues corresponding to amino acid residues 106 and 109 of the sequence of SEQ ID No: 14 has been substituted with amino acid residues different from cysteine.

In one embodiment, the substituted nucleotides of the nucleic acid sequence encoding the C_{H} region of a nucleic acid construct of the invention are substituted by using site-directed mutagenesis.

In one embodiment, a nucleic acid construct comprising a nucleic acid sequence encoding the C_{H} region of an IgG4, wherein the nucleic acid sequence encoding the C_{H} region has been modified such that the region corresponding to the hinge region does not comprise any amino acid residues capable of participating in the formation of disulphide bonds, is fused with a nucleic acid comprising a nucleic acid sequence encoding the V_{H} region of the monovalent antibody of the invention.

Thus, in one embodiment, the nucleic acid construct comprises a nucleic acid sequence encoding the V_{H} region of an antigen specific antibody, or a sequence complementary thereof.

In one embodiment, the nucleic acid sequence encoding the V_{H} region of the nucleic acid construct is operably linked to the nucleic acid sequence encoding the C_{H} region, or a sequence complementary thereof.

In one embodiment, the nucleic acid construct comprises a nucleotide sequence encoding the heavy chain of a monovalent immunoglobulin of the invention.

This may be achieved by using well-known technologies to obtain a nucleic acid construct wherein two different coding sequences are operably linked together. The nucleic acid sequence encoding the V_{H} region of a monovalent antibody of the invention may be derived from nucleic acids encoding the V_{H} region of any antigen specific antibody. In one embodiment, the V_{H} region is derived from the same antibody from which the V_{L} region of the monovalent antibody is derived from.

In one embodiment, the V_{H} and V_{L} regions recognize one end of a linker molecule, which is capable of binding the first molecule of the fusion protein with its other end. The invention provides examples of how to make nucleic acid constructs comprising
1) the nucleic acid sequence encoding the V_{H} of HuMab-7D8 identified as SEQ ID No: 5 and the nucleic acid sequence encoding the hingeless C_{H} of IgG4 identified as SEQ ID No: 15, wherein said sequences are operably linked together, and
2) the nucleic acid sequence encoding the V_{H} of mouse anti-Betv-1 identified as SEQ ID No: 7 and the nucleic acid sequence encoding the hingeless C_{H} of IgG4 identified as SEQ ID No: 15, wherein said sequences are operably linked together.

In one embodiment, the nucleic acid construct of the invention also comprises a nucleic acid sequence encoding the light chain of a monovalent immunoglobulin of the invention.

In one embodiment, a nucleic acid construct of the invention comprises a nucleic acid sequence encoding the V_{L} region of a monovalent immunoglobulin of the invention.

In one embodiment, a nucleic acid construct of the invention comprises a nucleic acid sequence encoding the C_{L} region of a monovalent antibody of the invention. In one embodiment, the C_{L} region is the C_{L} region of Ig light chain kappa. In one embodiment, the C_{L} region has the sequence of SEQ ID No: 1. In another embodiment, the C_{L} region is the C_{L} region of Ig light chain kappa. In one embodiment, the C_{L} region has the sequence of SEQ ID No: 3.

Such nucleic acid construct may be prepared by any known recombinant technology discussed herein, or prepared according to the procedures described in the present application in provided examples.

The nucleic acid sequence encoding the V_{L} region of the monovalent antibody of the invention may be derived from nucleic acids encoding the V_{H} region of any antigen specific antibody. In one embodiment, the V_{L} region is derived from the same antibody from which the V_{H} region of the monovalent antibody is derived from. The invention provides examples of how to make
1) a nucleic acid construct comprising the nucleic acid sequence encoding the V_{L} of HuMab-7D8 identified as SEQ ID No: 9 and the nucleic acid sequence encoding a C_{L} kappa of an Ig identified as SEQ ID No: 1, wherein said sequences are operably linked together, and
2) a nucleic acid construct comprising the nucleic acid sequence encoding the V_{L} of mouse anti-Betv-1 identified as SEQ ID No: 11 and the nucleic acid sequence encoding a C_{L} kappa of an Ig identified as SEQ ID No: 1, wherein said sequences are operably linked together.

The nucleic acids may be present in whole cells, in a cell lysate, or in a partially purified or substantially pure form.

The nucleic acid constructs of the present invention, while often in a native sequence (except for modified restriction sites and the like), from either cDNA, genomic or mixtures thereof, may be mutated in accordance with standard techniques to provide gene sequences. For coding sequences, these mutations, may affect amino acid sequence as desired. In particular, DNA sequences substantially homologous to or derived from native V, D, J, constant, switch variants and other such sequences described herein are contemplated (where “derived” indicates that a sequence is identical or modified from another sequence).

In one embodiment, the nucleic acid construct is a DNA construct. In one embodiment, the nucleic acid construct is a double-stranded DNA construct.

In one embodiment, the nucleic acid construct is a RNA construct.

In one embodiment, the fusion protein of the invention are prepared by allowing a nucleic acid construct as described above to be expressed in a cell.

Thus, the invention relates to a nucleic acid construct as described above, which is an expression vector. In one embodiment, the expression vector is a prokaryotic expression vector. In one embodiment, the expression vector is a eukaryotic expression vector. In one embodiment, the expression vector is a mammalian expression vector. Examples of different expression vectors, which may be used for the purpose of the invention, are discussed elsewhere herein and particular examples are described in the Example section. The invention provides a method of preparing a fusion protein of the invention comprising culturing a host cell comprising a nucleic acid construct of the invention, and, if said nucleic acid construct does not encode the light chain of the immunoglobulin of said fusion protein (if a light chain is present), also comprising a nucleic acid construct comprising a nucleic acid sequence encoding the light chain of said immunoglobulin, so that polypeptides are expressed, and recovering the polypeptides from the cell culture. In one embodiment, the fusion protein is recovered from the cell lysate. In another embodiment, the fusion protein is recovered from the cell culture medium.

The invention also provides the use of a nucleic acid construct of the invention for the production of a fusion protein of the invention or for the production of the different polypeptides that are part of the fusion protein. In one embodiment, said production includes the use of a method as described in further detail below.

A fusion protein or the polypeptides that are part of the fusion protein of the invention may thus for instance be prepared by expressing an expression vector comprising a nucleic acid sequence encoding the one polypeptide of the fusion protein of the invention and an expression vector comprising a nucleic sequence encoding an other polypeptide of the fusion protein of the invention, or an expression vector comprising both, in host cells. The host cells may be selected from any cells suitable for expression of foreign proteins, for example mammalian cells, as described elsewhere herein. The invention relates to both in vivo and in vitro expression.

For transient in vitro expression mammalian HEK293 cells may be used. In this case cells in culture are to be transfected with the expressions vectors of above by any suitable methods for cell transfection which are well-known in the art, for example a suitable cell transfection kit may be purchased from a commercial manufacturer, for example Stratagene or Invitrogene. For in vivo expression the expression vector is administered in vivo by any suitable way of administration developed for this purpose. The methods for administration of the expression vectors in vivo are also well known in the art.

Accordingly, the invention provides a host cell comprising a nucleic acid construct as described above. In one embodiment, the host cell is a prokaryotic cell. In one embodiment, the host cell is an E. coli cell. In another embodiment, the host cell is a eukaryotic cell. In one embodiment, the host cell is a mammalian cell. In one embodiment, the host cell is a CHO cell. In another embodiment, the host cell is a HEK-293F cell.

The invention also provides the use of a host cell of the invention for the production of a fusion protein of the invention. In one embodiment, said production includes the use of a method as described in further detail below. In one embodiment, the monovalent antibody is recovered from the cell lysate. In another embodiment, the monovalent antibody is recovered from the cell culture medium.

The invention also provides a transgene animal comprising a nucleic acid construct as described above.

In one embodiment, the nucleic acid sequence encoding the heavy chain of the monovalent immunoglobulin of the fusion protein has been modified such that the region corresponding to the hinge region of the heavy chain does not comprise any cysteine residues as described above.

In one embodiment, the nucleic acid sequence encoding the heavy chain has been modified such that at least one of the amino acid residues of the region corresponding to the hinge region, including any cysteine residues, have been deleted and/or substituted with other amino acid residues as described above.

In one embodiment, the nucleic acid sequence encoding the heavy chain has been modified such that the heavy chain comprises a C_{H} region, wherein the amino acids corresponding to amino acids 106 and 109 of the sequence of SEQ ID No: 14 have been deleted as described above.

In one embodiment, the nucleic acid sequence encoding the heavy chain has been modified such that the heavy chain comprises a C_{H} region, wherein at least the amino acid residues corresponding to amino acid residues 106 to 109 of the sequence of SEQ ID No: 14 has been deleted as described above.

In one embodiment, the nucleic acid sequence encoding the heavy chain has been modified such that the heavy chain comprises a C_{H} region, wherein at least the amino acid residues corresponding to amino acid residues 99 to 110 of the sequence of SEQ ID No: 14 has been deleted as described above.

In one embodiment, the nucleic acid sequence encoding the heavy chain has been modified such that the entire hinge region has been deleted as described above.

In one embodiment, the nucleic acid construct encoding the heavy chain of said monovalent antibody comprises a nucleotide sequence encoding a C_{H} region of a human IgG4, wherein at least one nucleotide of the splice donor site of the nucleic acid sequence encoding the hinge region has been substituted with another nucleotide as described above.

In one embodiment, the nucleic acid construct encoding the heavy chain of said monovalent antibody comprises a nucleotide sequence encoding a C_{H} region of a human IgG4, wherein the nucleotides corresponding to the nucleotides in position 714 and 722 of the sequence of SEQ ID No: 13 has been substituted with a nucleotide different than the nucleotide present at that position in SEQ ID No: 13 as described above.

In one embodiment, the nucleic acid construct encoding the heavy chain of said monovalent antibody comprises a nucleotide sequence encoding a C_{H} region of a human IgG4 comprising a sequence of SEQ ID No: 13, wherein nucleotides 714 and 722 of the sequence of SEQ ID No: 13 has been substituted with a nucleotide different than the nucleotide present at that position in SEQ ID No: 13 as described above.

In one embodiment, the nucleic acid construct encoding the heavy chain of said monovalent antibody comprises the nucleotide sequence of SEQ ID No: 15 as described above.

In one embodiment, the nucleic acid sequence encoding the heavy chain has been modified such that the heavy chain comprises a C_{H} region, wherein the amino acid residues corresponding to amino acid residues 106 and 109 of the sequence of SEQ ID No: 14 has been substituted with amino acid residues different from cysteine as described above.

In one embodiment, the substituted nucleotides of the nucleic acid sequence encoding the hinge region of the C_{H} region are substituted by using site-directed mutagenesis as described above.

In one embodiment, the nucleic acid construct encoding the light chain of said monovalent antibody comprises a sequence encoding the C_{L} region of the kappa chain of human IgG as described above.

In one embodiment, the nucleic acid construct comprises the nucleotide sequence of SEQ ID No: 1 as described above.

In one embodiment, the nucleic acid construct encoding the light chain of said monovalent antibody comprises a sequence encoding the C_{L} region of the lambda chain of human IgG as described above.

In one embodiment, the nucleic acid construct comprises the nucleotide sequence of SEQ ID No: 3 as described above.

In one embodiment, the nucleic acid constructs are DNA constructs as described above.

In one embodiment, the nucleic acid construct of comprising the sequences encoding the polypeptides of the fusion protein is a prokaryotic expression vector as described above. In a further embodiment, the cell expression system is a prokaryotic cell expression system as described above. In a further embodiment, the prokaryotic cell expression system comprises E. coli cells as described above. In a further embodiment, the E. coli cells are of a strain deficient in endogenous protease activities as described above.

In one embodiment, the nucleic acid construct of comprising the sequences encoding the polypeptides of the fusion protein is a eukaryotic expression vector as described above. In a further embodiment, the cell expression system is a eukaryotic cell expression system as described above. In a further embodiment, the cell expression system is a mammalian cell expression system as described above. In a further embodiment, the mammalian cell expression system comprises CHO cells as described above. In another further embodiment, the mammalian cell expression system comprises HEK-293F cells as described above.

The fusion proteins of the present invention have numerous in vitro and in vivo diagnostic and therapeutic utilities involving the diagnosis and treatment of disorders involving cells expressing the endogenous target recognized by the first molecule of the fusion protein. The invention does not relate to fusion proteins directed at any specific antigen, as according to the invention the fusion proteins described in the present specification may be made against any specific target.

In certain pathological conditions, it is necessary and/or desirable to utilize fusion proteins of the invention. Also, in some instances, it is preferred that a therapeutic fusion protein effects its therapeutic action without involving immune system-mediated activities, such as the effector functions, ADCC, phagocytosis and CDC. In such situations, it is desirable to generate forms of fusion proteins in which such activities are substantially reduced or eliminated. It is also advantageous if the fusion protein is of a form that can be made efficiently and with high yield. The present invention provides such fusion proteins, which may be used for a variety of purposes, for example as therapeutics, prophylactics and diagnostics.

The specific utility of a fusion protein of the invention is naturally dependent on the specific target of the fusion protein. Especially the advantages of not having effector functions of the immunoglobulin part of the fusion protein, and of having an extended half life of the first molecule (such information is abundant in the art regarding a host of different targets) is well within the skills of the person skilled in the art to evaluate.

In one embodiment, a fusion protein of the invention may act as an agonist of a particular cellular receptor, thereby potentiating, enhancing or activating either all or partial activities of the ligand-mediated receptor activation.

In one embodiment, a fusion protein of the invention may prevent binding of a virus or other pathogen to its receptor, such as inhibition of HIV binding to CD4 or coreceptor such as CCR5 or CXCR4.

In one embodiment, a fusion protein of the invention may be used to treat, inhibit, delay progression of, prevent/delay recurrence of, ameliorate, or prevent diseases, disorders or conditions associated with abnormal expression and/or activity of one or more antigen molecules, such as including but not limited to malignant and benign tumors; non-leukemias and lymphoid malignancies; neuronal, glial, astrocytal, hypothalamic and other glandular, macrophagel, epithelial, stromal and blastocoelic disorders; and inflammatory, angiogenic and immunologic disorders.

In one embodiment, a fusion protein of the invention may be used to treat, such as inhibit, delay progression of, prevent/delay recurrence of, or ameliorate, or to prevent diseases, disorders or conditions such as a cancer, a cell proliferative disorder, an (auto-) immune disorder, an inflammation disorder and/or an angiogenesis disorder. This will depend on the fusion proteins being able to, through its target specificity, to interfere with cell proliferation, cell growth, cell viability, apoptosis, necrosis, cell-cell interaction, cell-matrix interaction, cell signaling, cell-surface molecule expression, cell-surface molecule interactions, ligand-receptor interactions.

The present invention provides a fusion protein of the invention for use as a medicament.

The present invention provides a fusion protein of the invention for use as a medicament for treating cancer, a cell proliferative disorder, an (auto-) immune disorder, an inflammation disorder and/or an angiogenesis disorder, wherein the fusion protein specifically binds a given target or target epitope, where the binding of a fusion protein to said target or target epitope is effective in treating said disease.

The present invention provides a fusion protein of the invention for use as a medicament for treating a disease or disorder, which disease or disorder is treatable by administration of an fusion protein against a certain target, wherein the involvement of immune system-mediated activities is not necessary or is undesirable for achieving the effects of the administration of the antibody, and wherein said antibody specifically binds said antigen.

The present invention provides a fusion protein of the invention for use as a medicament for treating a disease or disorder, which disease or disorder is treatable by administration of the first molecule with an extended half life.

The present invention provides the use of a fusion protein of the invention as a medicament.

The present invention provides the use of a fusion protein of the invention as a medicament for treating cancer, a cell proliferative disorder, an (auto-) immune disorder, an inflammation disorder and/or an angiogenesis disorder, wherein the fusion protein specifically binds a given target or target epitope, where the binding of a fusion protein to said target or target epitope is effective in treating said disease.

The present invention provides the use of a fusion protein of the invention as a medicament for treating a disease or disorder, which disease or disorder is treatable by blocking or inhibiting a soluble antigen, wherein multimerization (such as dimerization) of said antigen may form undesirable immune complexes.

The present invention provides the use of a fusion protein of the invention as a medicament for treating a disease or disorder, which disease or disorder is treatable by administration of an fusion protein against a certain target, wherein the involvement of immune system-mediated activities is not necessary or is undesirable for achieving the effects of the administration of the fusion protein, and wherein said fusion protein specifically binds said target.

The present invention provides the use of a fusion protein of the invention as a medicament for treating a disease or disorder, which disease or disorder is treatable by blocking or inhibiting a cell membrane bound receptor, wherein said receptor may be activated by dimerization of said receptor, and wherein said fusion protein specifically binds said receptor.

The present invention provides the use of a fusion protein of the invention for the preparation of a pharmaceutical composition for treating cancer, a cell proliferative disorder, an (auto-) immune disorder, an inflammation disorder and/or an angiogenesis disorder, wherein the fusion protein specifically binds a given target or target epitope, where the binding of an fusion protein to said target or target epitope is effective in treating said disease.

The present invention provides the use of a fusion protein of the invention for the preparation of a pharmaceutical composition for treating a disease or disorder, which disease or disorder is treatable by blocking or inhibiting a soluble target molecule, wherein multimerization (such as dimerization) of said target molecule may form undesirable immune complexes.

The present invention provides the use of a fusion protein of the invention for the preparation of a pharmaceutical composition for treating a disease or disorder, which disease or disorder is treatable by administration of a fusion protein against a certain target, wherein the involvement of immune system-mediated activities is not necessary or is undesirable for achieving the effects of the administration of the fusion protein, and wherein said fusion protein specifically binds said target molecule.

The present invention provides the use of a fusion protein of the invention for the preparation of a pharmaceutical composition for treating a disease or disorder, which disease or disorder is treatable by blocking or inhibiting a cell membrane bound receptor, wherein said receptor may be activated by dimerization of said receptor, and wherein said fusion protein specifically binds said receptor.

The invention provides a method of treating a disease or disorder, wherein said method comprises administering to a subject in need of treatment a fusion protein of the invention, a pharmaceutical composition comprising said fusion protein, immunoconjugate comprising said fusion protein, or a nucleic acid construct of the invention, whereby the disease or disorder is treated.

The invention provides a method for inhibiting a target protein in a subject suffering from a disease or disorder in which activity of the target protein is undesirable, comprising administering to a subject in need of treatment a therapeutically effective amount of a fusion protein of the invention, which fusion protein specifically binds said target protein, a pharmaceutical composition comprising said fusion protein, immunoconjugate comprising said fusion protein, or a nucleic acid construct of the invention, such that the target protein activity in the subject is inhibited.

The present invention provides a method of treating cancer, a cell proliferative disorder, an (auto)immune disorder, an inflammation disorder and/or an angiogenesis disorder, wherein said method comprises administering to a subject in need of treatment a therapeutically effective amount of a fusion protein of the invention, a pharmaceutical composition comprising said fusion protein, immunoconjugate comprising said fusion protein, or a nucleic acid construct of the invention, and wherein the fusion protein specifically binds a given target or target epitope, where the binding of an fusion protein to said target or target epitope is effective in treating said disease.

In one embodiment, such disease or disorder is a disease or disorder treatable by blocking or inhibiting a soluble antigen, wherein multimerization (such as dimerization) of said antigen may form undesirable immune complexes, comprising administering to a subject in need of treatment a therapeutically effective amount of a fusion protein of the invention directed at said target protein, a pharmaceutical composition comprising said fusion protein, immunoconjugate comprising said fusion protein, or a nucleic acid construct of the invention.

In one embodiment, such disease or disorder is a disease or disorder treatable by administration of an fusion protein against a certain target, wherein the involvement of immune system-mediated activities is not necessary or is undesirable for achieving the effects of the administration of the fusion protein, comprising administering to a subject in need of treatment a therapeutically effective amount of a fusion protein of the invention, which fusion protein specifically binds said target protein, a pharmaceutical composition comprising said fusion protein, immunoconjugate comprising said fusion protein, or a nucleic acid construct of the invention.

In one embodiment, such disease or disorder is a disease or disorder treatable by blocking or inhibiting a cell membrane bound receptor, wherein said receptor may be activated by dimerization of said receptor, comprising administering to a subject in need of treatment a therapeutically effective amount of a monovalent fusion protein of the invention, which fusion protein specifically binds said receptor, a pharmaceutical composition comprising said fusion protein, immunoconjugate comprising said fusion protein, or a nucleic acid construct of the invention.

The scientific literature is abundant with examples of targets, where the binding of ligands or inhibitors against said target, is shown to have, or is expected to have, a therapeutic effect. Given the teaching of this specification and as described elsewhere herein, it is within the skill of a person skilled in the art to determine, whether the use of a fusion protein, such as a fusion protein of the present invention, against such targets would be expected to produce an improved therapeutic effect due to a longer half life of the first molecule of the fusion protein. In the following, several examples of such targets are given; however, these examples are not meant to be construed as limiting for the scope of the invention.

Fusion protein of the invention may be used either alone or in combination with other compositions in a therapy. For instance, a fusion protein of the invention may be co-administered with one or more antibodies, such as monovalent antibodies, one or more chemotherapeutic agent(s) (including cocktails of chemotherapeutic agents), one or more other cytotoxic agent(s), one or more anti-angiogenic agent(s), one or more cytokines, one or more growth inhibitory agent(s), one or more anti-inflammatory agent(s), one or more disease modifying antirheumatic drug(s) (DMARD), or one or more immunosuppressive agent(s), depending on the disease or condition to be treated. Where a fusion protein of the invention inhibits tumor growth, it may be particularly desirable to combine it with one or more other therapeutic agent(s) which also inhibits tumor growth. Alternatively, or additionally, the patient may receive combined radiation therapy (for instance external beam irradiation or therapy with a radioactive labeled agent, such as an antibody). Such combined therapies noted above include combined administration (where the two or more agents are included in the same or separate formulations), and separate administration, in which case, administration of the fusion protein of the invention may occur prior to, and/or following, administration of the adjunct therapy or therapies.

A fusion protein composition of the invention may be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. In one embodiment, the fusion protein may be formulated with one or more agents currently used to prevent or treat the disorder in question. The effective amount of such other agents depends on the amount of fusion proteins of the invention present in the formulation, the type of disorder or treatment, and other factors discussed above.

The fusion protein of the invention (and adjunct therapeutic agent) may be administered by any suitable means, including parenteral, subcutaneous, intraperitoneal, intrapulmonary, and intranasal, and, if desired for local treatment, intralesional administration. Parenteral infusions include intramuscular, intravenous, intraarterial, intraperitoneal, or subcutaneous administration. In addition, the fusion protein may be suitably administered by pulse infusion, particularly with declining doses of the fusion protein. Dosing may be by any suitable route, for instance by injections, such as intravenous or subcutaneous injections, depending in part on whether the administration is brief or chronic.

For the prevention or treatment of disease, the appropriate dosage of a fusion protein of the invention (when used alone or in combination with other agents such as chemotherapeutic agents) will depend on the type of disease to be treated, the type of fusion protein, the severity and course of the disease, whether the fusion protein is administered for preventive, therapeutic or diagnostic purposes, previous therapy, the patient's clinical history and response to the fusion protein, and the discretion of the attending physician. The fusion protein may be suitably administered to the patient at one time or over a series of treatments.

Such dosages may be administered intermittently, for instance every week or every three weeks (for instance such that the patient receives from about two to about twenty, for instance about six doses of the fusion protein). An initial higher loading dose, followed by one or more lower doses may be administered. An exemplary dosing regimen comprises administering an initial loading dose of about 4 mg/kg, followed by a weekly maintenance dose of about 2 mg/kg of the fusion protein. However, other dosage regimens may be useful. In one embodiment, the fusion protein of the invention are administered in a weekly dosage of from 50 mg to 4000 mg, for instance of from 250 mg to 2000 mg, such as for example 300 mg, 500 mg, 700 mg, 1000 mg, 1500 mg or 2000 mg, for up to 8 times, such as from 4 to 6 times. The weekly dosage may be divided into two or three subdosages and administered over more than one day. For example, a dosage of 300 mg may be administered over 2 days with 100 mg on day one (1), and 200 mg on day two (2). A dosage of 500 mg may be administered over 3 days with 100 mg on day one (1), 200 mg on day two (2), and 200 mg on day three (3), and a dosage of 700 mg may be administered over 3 days with 100 mg on day 1 (one), 300 mg on day 2 (two), and 300 mg on day 3 (three). The regimen may be repeated one or more times as necessary, for example, after 6 months or 12 months.

The dosage may be determined or adjusted by measuring the amount of circulating fusion protein of the invention upon administration in a biological sample for instance by using anti-idiotypic antibodies which target said fusion protein (if a variable part of the immunoglobulin of the fusion protein is present). In case the fusion protein does not comprise any variable regions of the immunoglobulin, antibodies may be raised against an other part of the fusion protein, for use in quantitation measurements.

In one embodiment, the fusion protein of the invention may be administered by maintenance therapy, such as, for instance once a week for a period of 6 months or more.

In one embodiment, the fusion protein of the invention may be administered by a regimen including one infusion of a fusion protein of the invention followed by an infusion of same fusion protein conjugated to a radioisotope. The regimen may be repeated, for instance 7 to 9 days later.

The progress of this therapy may be monitored by conventional techniques and assays.

The invention provides an article of manufacture containing materials useful for the treatment, prevention and/or diagnosis of the disorders described above. An article of manufacture of the present invention comprises a container and a label or package insert on or associated with the container. Suitable containers include, for example, bottles, vials, syringes, etc. The containers may be formed from a variety of materials such as glass or plastic. The container holds a composition which is by itself or when combined with other compositions effective for treating, preventing and/or diagnosing the condition and may have a sterile access port (for example the container may be an intravenous solution bag or a vial having a stopper pierceable by a hypodermic injection needle). At least one active agent in the composition is a fusion protein of the invention. The label or package insert indicates that the composition is used for treating the condition of choice, for instance cancer. Moreover, the article of manufacture may comprise (a) a first container with a composition contained therein, wherein the composition comprises a fusion protein of the invention; and (b) a second container with a composition contained therein, wherein the composition comprises a further cytotoxic agent. The article of manufacture in this embodiment of the invention may further comprise a package insert indicating that the first and second composition may be used to treat a particular condition, for instance cancer. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Also within the scope of the present invention are kits comprising pharmaceutical compositions of the invention comprising one or more fusion proteins of the invention and instructions for use. The kit may further comprise one or more additional agents, such as an immunosuppressive reagent, a cytotoxic agent or a radiotoxic agent, depending on the disease or disorder to be treated, or one or more additional fusion proteins of the invention (for instance a fusion protein having a complementary activity).

In one embodiment, the invention provides methods for detecting the presence of the specific antigen to which the fusion protein binds, in a sample, or measuring the amount of said specific target protein, comprising contacting the sample, and a control sample, with a fusion protein, which specifically binds to said target protein, under conditions that allow for formation of a complex between the fusion protein or portion thereof and said target protein. The formation of a complex is then detected, wherein a difference complex formation between the sample compared to the control sample is indicative the presence of said antigen in the sample.

In one embodiment, fusion proteins of the invention may be used to detect levels of circulating specific target protein to which the fusion protein binds, or levels of cells which contain said specific target protein, on their membrane surface, which levels may then be linked to certain disease symptoms. Alternatively, the fusion proteins may be used to deplete or interact with the function of cells expressing said target protein, thereby implicating these cells as important mediators of the disease. This may be achieved by contacting a sample and a control sample with the fusion protein under conditions that allow for the formation of a complex between the fusion protein and said specific target protein. Any complexes formed between the fusion protein and said antigen are detected and compared in the sample and the control.

In one embodiment, the invention provides a method for detecting the presence or quantifying, in vivo or in vitro, the amount of cells expressing the specific target protein to which the fusion protein binds. The method comprises (i) administering to a subject a fusion protein of the invention conjugated to a detectable marker; (ii) exposing the subject to a means for detecting said detectable marker to identify areas containing cells expressing said antigen.

### EXAMPLES

### Example 1

### Oligonucleotide Primers and PCR Amplification

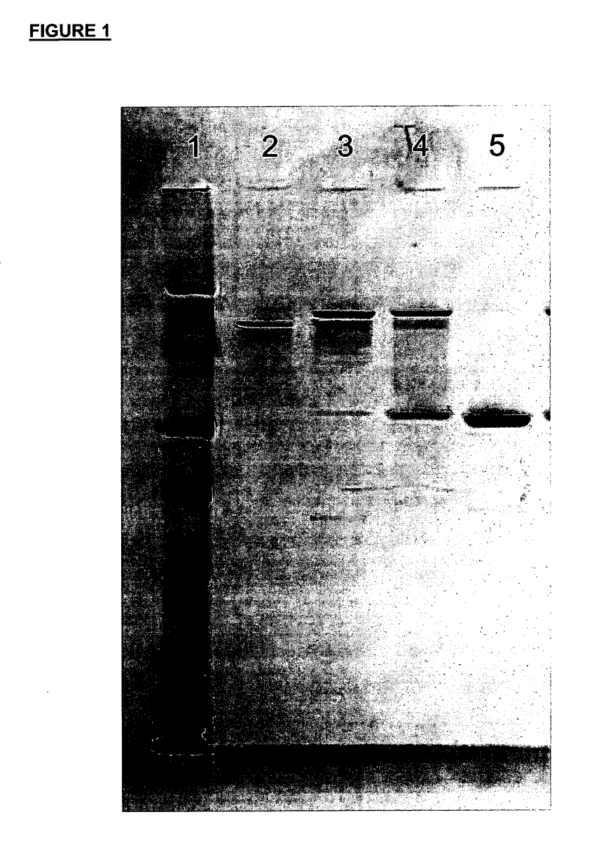

Oligonucleotide primers were synthesized and quantified by Isogen Bioscience (Maarssen, The Netherlands). Primers were dissolved in H₂O to 100 pmol/μl and stored at −20° C. A summary of all PCR and sequencing primers is tabulated (FIG. 1). For PCR, PfuTurbo® Hotstart DNA polymerase (Stratagene, Amsterdam, The Netherlands) was used according to the manufacturer's instructions. Each reaction mix contained 200 μM mixed dNTPs (Roche Diagnostics, Almere, The Netherlands), 6.7 pmol of both the forward and reverse primer, 100 ng of genomic DNA or 1 ng of plasmid DNA and 1 unit of PfuTurbo® Hotstart DNA polymerase in PCR reaction buffer (supplied with polymerase) in a total volume of 20 μl. PCR reactions were carried out with a TGradient Thermocycler 96 (Whatman Biometra, Goettingen, Germany) using a 32-cycle program: denaturing at 95° C. for 2 min; 30 cycles of 95° C. for 30 sec, a 60-70° C. gradient (or another specific annealing temperature) for 30 sec, and 72° C. for 3 min; final extension at 72° C. for 10 min. If appropriate, the PCR mixtures were stored at 4° C. until further analysis or processing.

### Example 2

### Agarose Gel Electrophoresis

Agarose gel electrophoresis was performed according to Sambrook (Sambrook J. and Russel, D. V. Molecular Cloning: A Laboratory Manual, 3nd Ed., Cold Spring Harbor, 2000) using gels of 50 ml, in 1×Tris Acetate EDTA buffer. DNA was visualized by the inclusion of ethidium bromide in the gel and observation under UV light. Gel images were recorded by a CCD camera and an image analysis system (GeneGnome; Syngene, via Westburg B.V., Leusden, The Netherlands).

### Example 3

### Analysis and Purification of PCR Products and Enzymatic Digestion Products

Purification of desired PCR fragments was carried out using a MinElute PCR Purification Kit (Qiagen, via Westburg, Leusden, The Netherlands; product#28006), according to the manufacturer's instructions. Isolated DNA was quantified by UV spectroscopy and the quality was assessed by agarose gel electrophoresis.

Alternatively, PCR or digestion products were separated by agarose gel electrophoresis (for instance when multiple fragments were present) using a 1% Tris Acetate EDTA agarose gel. The desired fragment was excised from the gel and recovered using the QIAEX II Gel Extraction Kit (Qiagen; product#20051), according to the manufacturer's instructions.

### Example 4

### Quantification of DNA by UV Spectroscopy

Optical density of nucleic acids was determined using a NanoDrop ND-1000 Spectrophotometer (Isogen Life Science, Maarssen, The Netherlands) according to the manufacturer's instructions. The DNA concentration was measured by analysis of the optical density (OD) at 260 nm (one OD₂₆₀ₙₘ unit=50 μg/ml). For all samples, the buffer in which the nucleic acids were dissolved was used as a reference.

### Example 5

### Restriction Enzyme Digestions

Restriction enzymes and supplements were obtained from New England Biolabs (Beverly, Mass., USA) or Fermetas (Vilnius, Lithuania) and used according to the manufacturer's instructions.

DNA (100 ng) was digested with 5 units of enzyme(s) in the appropriate buffer in a final volume of 10 μl (reaction volumes were scaled up as appropriate). Digestions were incubated at the recommended temperature for a minimum of 60 min. For fragments requiring double digestions with restriction enzymes which involve incompatible buffers or temperature requirements, digestions were performed sequentially. If necessary digestion products were purified by agarose gel electrophoresis and gel extraction.

### Example 6

### Ligation of DNA Fragments

Ligations of DNA fragments were performed with the Quick Ligation Kit (New England Biolabs) according to the manufacturer's instructions. For each ligation, vector DNA was mixed with approximately three-fold molar excess of insert DNA.

### Example 7

### Transformation of E. coli

Plasmid DNA (1-5 μl of DNA solution, typically 2 μl of DNA ligation mix) was transformed into One Shot DH5α-T1^{R} or MACH-1 T1^{R} competent E. coli cells (Invitrogen, Breda, The Netherlands; product#12297-016) using the heat-shock method, according to the manufacturer's instructions. Next, cells were plated on Luria-Bertani (LB) agar plates containing 50 μg/ml ampicillin. Plates were incubated for 16-18 hours at 37° C. until bacterial colonies became evident.

### Example 8

### Screening of Bacterial Colonies by PCR

Bacterial colonies were screened for the presence of vectors containing the desired sequences via colony PCR using the HotStarTaq Master Mix Kit (Qiagen; product#203445) and the appropriate forward and reverse primers. Selected colonies were lightly touched with a 20 μl pipette tip and touched briefly in 2 ml LB for small scale culture, and then resuspended in the PCR mix. PCR was performed with a TGradient Thermocycler 96 using a 35-cycle program: denaturation at 95° C. for 15 min; 35 cycles of 94° C. for 30 sec, 55° C. for 30 sec and 72° C. for 2 min; followed by a final extension step of 10 min at 72° C. If appropriate, the PCR mixtures were stored at 4° C. until analysis by agarose gel electrophoresis.

### Example 9

### Plasmid DNA Isolation from E. coli Culture

Plasmid DNA was isolated from E. coli cultures using the following kits from Qiagen (via Westburg, Leusden, The Netherlands), according to the manufacturer's instructions. For bulk plasmid preparation (50-150 ml culture), either a HiSpeed Plasmid Maxi Kit (product#12663) or a HiSpeed Plasmid Midi Kit (product#12643) was used. For small scale plasmid preparation (±2 ml culture) a Qiaprep Spin Miniprep Kit (product#27106) was used and DNA was eluted in 50 μl elution buffer (supplied with kit).

### Example 10

### Site-Directed Mutagenesis

Site-directed mutagenesis was performed using the QuickChange II XL Site-Directed Mutagenesis Kit (Stratagene, Amsterdam, The Netherlands) according to the manufacturer's instructions. This method included the introduction of a silent extra XmaI site to screen for successful mutagenesis. Briefly, 5 μl 10× reaction buffer, 1 μl oligonucleotide IgG4S228Pf (P16) (100 pmol/μl), 1 μl oligonucleotide IgG4S228Pr (P17) (100 pmol/μl), 1 μl dNTP mix, 3 μl Quicksolution, 1 μl plasmid pTomG4Tom7D8 (see example 16) (50 ng/μl) and 1 μl PfuUltra HF DNA polymerase were mixed in a total volume of 50 μl and amplified with a TGradient Thermocycler 96 (Whatman Biometra, Goettingen, Germany; product#050-801) using an 18-cycle program: denaturing at 95° C. for 1 min; 18 cycles of 95° C. for 50 sec, 60° C. for 50 sec, and 68° C. for 10 min. PCR mixtures were stored at 4° C. until further processing. Next, PCR mixtures were incubated with 1 μl DpnI for 60 min at 37° C. to digest the pTomG47D8 vector and stored at 4° C. until further processing. The reaction mixture was precipitated with 5 μl sM NaAc and 125 μl Ethanol, incubated for 20 minutes at −20° C. and spundown for 20 minutes at 4° C. at 14000×g. The DNA pellet was washed with 70% ethanol, dried and dissolved in 4 μl water. The total 4 μl reaction volume was transformed in One Shot Top 10 competent E. coli cells (Invitrogen, Breda, The Netherlands) according to the manufacturer's instructions (Invitrogen). Next, cells were plated on Luria-Bertani (LB) agar plates containing 50 μg/ml ampicillin. Plates were incubated for 16-18 hours at 37° C. until bacterial colonies became evident.

### Example 11

### DNA Sequencing

Plasmid DNA samples were sent to AGOWA (Berlin, Germany) for sequence analysis. Sequences were analyzed using Vector NTI advanced software (Informax, Oxford, UK).

### Example 12

### Transient Expression in HEK-293F Cells

Freestyle™ 293-F (a HEK-293 subclone adapted to suspension growth and chemically defined Freestyle medium, e.g. HEK-293F) cells were obtained from Invitrogen and transfected according to the manufacturer's protocol using 293fectin (Invitrogen).

### Example 13

### Construction of pConG1fA77: A Vector for the Production of the Heavy Chain of A77-IgG1

The V_{H} coding region of the mouse anti-FcαRI antibody A77 was amplified from a scFv phage vector, containing the VH and VL coding regions of this antibody, by a double overlap extension PCR. This was used to incorporate a mammalian signal peptide, an ideal Kozak sequence and suitable restriction sites for cloning in pConG1f. The first PCR was done using primers A77VHfor1 and A77VHrev with the scFv phage vector as template. Part of this first PCR was used in a second PCR using primers A77VHfor2 and A77VHrev. The VH fragment was gel purified and cloned into pConG1f0.4. For this the pConG1f0.4 vector and the VH fragment were digested with HindIII and ApaI and purified. The V_{H} fragment and the pConG1f0.4HindIII-ApaI digested vector were ligated and transformed into competent DH5α-T1^{R} cells. A clone was selected containing the correct insert size and the sequence was confirmed and was named pConG1fA77.

### Example 14

### Construction of pConKA77: A Vector for the Production of the Light Chain of A77 Antibodies

The V_{L} coding region of the mouse anti-FcαRI antibody A77 was amplified from a scFv phage vector, containing the VH and VL of this antibody, by a double overlap extension PCR. This was used to incorporate a mammalian signal peptide, an ideal Kozak sequence and suitable restriction sites for cloning in pConKappa0.4. The first PCR was done using primers A77VLfor1 and A77VLrev with the scFv phage vector as template. Part of this first PCR was used in a second PCR using primers A77VLfor2 and A77VLrev. The PCR product and the pConKappa0.4 vector were digested with HindIII and Pfl123II and purified. The V_{L} fragment and the pConKappa0.4HindIII-Pfl23II digested vector were ligated and transformed into competent DH5α T1^{R} E. coli.

A clone was selected containing the correct insert size and the sequence was confirmed. This plasmid was named pConKA77.

### Example 15

### Construction of pTomG4A77: A Vector for the Production of the Heavy Chain of A77-IgG4

To construct a vector for expression of A77-IgG4, the VH region of A77 was cloned in pTomG4.

For this, pTomG4 and pConG1fA77 were digested with HindIII and ApaI and the relevant fragments were isolated.

The A77 V_{H} fragment and the pTomG4HindIII-ApaI digested vector were ligated and transformed into competent DH5α-T1^{R} cells.

A clone was selected containing the correct insert size. This plasmid was named pTomG4A77.

### Example 16

### Construction of pTomG4A77HG: A Vector for the Production of the Heavy Chain of A77-HG

To make a construct for expression of A77-HG, the VH region of A77 was cloned in pTomG47D8HG, replacing the VH 7D8 region.

For this pTomG47D8HG and pConG1fA77 were digested with HindIII and ApaI and the relevant fragments were isolated.

The A77 V_{H} fragment and the pTomG47D8HGHindIII-ApaI digested vector fragment were ligated and transformed into competent DH5α-T1^{R} cells.

A clone was selected containing the correct insert size. This plasmid was named pTomG4A77HG.

### Example 17

### Construction of pEE6.4A77Fab: A Vector for the Production of the Heavy Chain of A77-Fab

To make a construct for expression of A77-Fab, the VH region of A77 was cloned in pEE6.42F8Fab, replacing the VH 2F8 region.

For this pEE6.42F8Fab and pConG1fA77 were digested with HindIII and ApaI and the relevant fragments were isolated.

The A77 V_{H} fragment and the pEE6.42F8Fab HindIII-ApaI digested vector fragment were ligated and transformed into competent DH5α-T1^{R} cells.

A clone was selected containing the correct insert. This plasmid was named pEE6.4A77Fab.

### Example 18

### Cloning of the Variable Regions of a Human Anti-cMet Antibody

Total RNA was prepared from 1×10⁶ mouse hybridoma cells with the RNeasy kit (Qiagen, Westburg, Leusden, Netherlands) according to the manufacturer's protocol.

5′-RACE-Complementary DNA (cDNA) of RNA was prepared from 60 ng total RNA, using the SMART RACE cDNA Amplification kit (BD Biosciences Clontech, Mountain View, Calif., USA), following the manufacturer's protocol.

The VL and VH regions of the cMet antibody were amplified by PCR. For this PfuTurbo® Hotstart DNA polymerase (Stratagene) was used according to the manufacturer's instructions. Each reaction mix contained 5 μl 10×BD Advantage 2 PCR buffer (Clontech), 200 μM mixed dNTPs (Roche Diagnostics), 12 pmol of the reverse primer (RACEG1A1 for the VH region and RACEKA1 for the VL region), 7.2 pmol UPM-Mix (UPM-Mix: 2 μM ShortUPMH3 and 0.4 μM LongUPMH3 oligonucleotide), 1 μl of the 5′RACE cDNA template as described above, and 1 μl 50×BD Advantage 2 polymerase mix (Clontech) in a total volume of 50 μl.

PCR reactions were carried out with a TGradient Thermocycler 96 (Whatman Biometra) using a 35-cycle program: denaturing at 95° C. for 1 min; 35 cycles of 95° C. for 30 sec, 68° C. for 60 sec.

The reaction products were separated by agarose gel electrophoresis on a 1% TAE agarose gel and stained with ethidium bromide. Bands of the correct size were cut from the gels and the DNA was isolated from the agarose using the Qiagen Minelute Reaction Cleanup kit (Qiagen).

Gel isolated PCR fragments were cloned into the pCR4Blunt-TOPO vector (Invitrogen) using the Zero Blunt® TOPO® PCRCloning Kit for Sequencing (Invitrogen), following the manufacturer's protocol. 5 μl of the ligation mixture was transformed into OneShot DH5αT1R competent E. Coli (Invitrogen) and plated on LB/Ampicillin plates.

From six, insert containing, clones, the V_{L} sequences were determined and from five, insert containing, clones, the V_{H} sequences were determined.

### Example 19

### Construction of pConG1fcMet: A Vector for the Production of the Heavy Chain of cMet-IgG1

The V_{H} coding region of the human anti-cMet antibody was cut from a plasmid containing this region using HindIII and ApaI. The VH fragment was gel purified and cloned into pConG1f0.4. For this pConG1f0.4 vector were digested with HindIII and ApaI and purified. The V_{H} fragment and the pConG1f0.4HindIII-ApaI digested vector were ligated and transformed into competent DH5α-T1R cells.

A clone was selected containing the correct insert size was isolated and was named pConG1fcMet.

### Example 20

### Construction of pConKcMet: A Vector for the Production of the Light Chain of cMet Antibodies

The V_{L} coding region of the human anti-cMet antibody was amplified from a plasmid containing this region using the primers shortUPMH3 and RACEVLBsiWI, introducing suitable restriction sites for cloning into pConK0.4.

The PCR product and the pConKappa0.4 vector were digested with HindIII and Pfl23II and purified. The V_{L} fragment and the pConKappa0.4HindIII-Pfl23II digested vector were ligated and transformed into competent DH5α T1^{R} E. coli.

A clone was selected containing the correct insert size and the sequence was confirmed. This plasmid was named pConKcMet.

### Example 21

### Construction of pTomG4cMet: A Vector for the Production of the Heavy Chain of cMet-IgG4

To construct a vector for expression of cMet-IgG4, the VH region of cMet was cloned in pTomG4.

For this, pTomG42F8 and pConG1fcMet were digested with HindIII and ApaI and the relevant fragments were isolated.

The cMet V_{H} fragment and the pTomG42F8HindIII-ApaI digested vector were ligated and transformed into competent DH5α-T1^{R} cells.

A clone was selected containing the correct insert size. This plasmid was named pTomG4cMet.

### Example 22

### Construction of pTomG4cMetHG: A Vector for the Production of the Heavy Chain of cMet-HG

To make a construct for expression of cMet-HG, the VH region of cMet was cloned in pTomG42F8HG, replacing the VH 2F8 region.

For this pTomG42F8HG and pConG1fcMet were digested with HindIII and ApaI and the relevant fragments were isolated.

The cMet V_{H} fragment and the pTomG42F8HGHindIII-ApaI digested vector fragment were ligated and transformed into competent DH5α-T1^{R} cells.

A clone was selected containing the correct insert size. This plasmid was named pTomG4cMetHG.

### Example 23

### Construction of pEE6.4cMetFab: A Vector for the Production of the Heavy Chain of cMet-Fab

To make a construct for expression of cMet-Fab, the VH region of cMet was cloned in pEE6.42F8Fab, replacing the VH 2F8 region.

For this pEE6.42F8Fab and pConG1fcMet were digested with HindIII and ApaI and the relevant fragments were isolated.

The cMet V_{H} fragment and the pEE6.42F8Fab HindIII-ApaI digested vector fragment were ligated and transformed into competent DH5α-T1^{R} cells.

A clone was selected containing the correct insert. This plasmid was named pEE6.4cMetFab.

### Example 24

### Construction of pConG1f2F8: A Vector for the Production of the Heavy Chain of 2F8-IgG1

The V_{H} coding region of 2F8 (WO 2002/100348) was amplified by PCR from pIESRα2F8 (Medarex) using the primers 2f8HCexfor and 2f8HCexrev and subcloned in PCRscriptCam (Stratagene). The VH fragment was subsequently cloned in pCONg1f0.4. For this pConG1f0.4 and the pCRScriptCAMVH2F8 vectors were digested with HindIII and ApaI and the relevant fragments were purified.

The V_{H} fragment and the pConG1f0.4HindIII-ApaI digested vector were ligated and transformed into competent DH5α-T1^{R} cells. A clone was selected containing the correct insert size, the sequence was confirmed and the vector was named pConG1f2F8.

### Example 25

### Construction of pConK2F8: A Vector for the Production of the Light Chain of 2F8 Antibodies

pIESRα2F8 was digested with HindIII and BsiWI and the V_{L} coding region of 2F8 (anti-EGFr) was isolated from gel. The pConKappa0.4 vector was digested with HindIII and BsiWI and purified. The V_{L} fragment and the pConKappa0.4HindIII-BsiWI digested vector were ligated and transformed into competent DH5α T1^{R} E. coli.

A clone was selected containing the correct insert size and the sequence was confirmed. This plasmid was named pConK2F8.

### Example 26

### Construction of pTomG42F8: A Vector for the Production of the Heavy Chain of 2F8-IgG4

To construct a vector for expression of 2F8-IgG4, the VH region of 2F8 was cloned in pTomG4.

For this, pTomG4 and pConG1f2F8 were digested with HindIII and ApaI and the relevant fragments were isolated.

The 2F8 V_{H} fragment and the pTomG4HindIII-ApaI digested vector were ligated and transformed into competent DH5α-T1^{R} cells.

A clone was selected containing the correct insert size. This plasmid was named pTomG42F8.

### Example 27

### Construction of pTomG42F8HG: A Vector for the Production of the Heavy Chain of 2F8-HG

To make a construct for expression of 2F8-HG, the VH region of 2F8 was cloned in pTomG47D8HG, replacing the VH 7D8 region.

For this pTomG47D8HG and pConG1f2F8 were digested with HindIII and ApaI and the relevant fragments were isolated.

The 2F8 V_{H} fragment and the pTomG47D8HGHindIII-ApaI digested vector fragment were ligated and transformed into competent DH5α-T1^{R} cells.

A clone was selected containing the correct insert size. This plasmid was named pTomG42F8HG.

### Example 28

### Construction of pEE6.42F8Fab: A Vector for the Production of the Heavy Chain of 2F8-Fab

The Fab coding region was amplified from vector pConG1f2F8 by PCR with primers pConG1seq1 and 2F8fabrev2, introducing a suitable cloning restriction site and a C-terminal his tag coding sequence. The PCR fragment was purified and cloned in PEE6.4. For this pEE6.4 and the PCR fragment were digested with HindIII and EcoRI and the relevant fragments were isolated.

The 2F8 Fab fragment and the pEE6.4HindIII-EcoRI digested vector fragment were ligated and transformed into competent DH5α-T1^{R} cells.

A clone was selected containing the correct insert and the sequence was confirmed by DNA sequencing. This plasmid was named pEE6.42F8Fab.

### Example 29

### Construction of pConG1f7D8: A Vector for Production of the Heavy Chain of 7D8-IgG1

The V_{H} coding region of CD20 specific HuMab-7D8 (WO 04/035607) was amplified by PCR from a pGemT (Promega, Madison, USA) vector containing this region using the primers 7D8VHexfor (P8) and 2F8HCexrev (P13) (FIG. 14), introducing suitable restriction sites for cloning into pConG1f0.4 (Lonza Biologics, Slough, UK), a mammalian expression vector containing the genomic constant region (allotype f) of human IgG1, and an ideal Kozak sequence (GCCGCCACC, (Kozak M et al., Gene 234(2), 187-208 (1999)). The PCR fragment was cloned in pPCR-Script CAM (Stratagene, Amsterdam, The Netherlands) using a PCR-Script® Cam Cloning Kit (Stratagene), according to the manufacture's instructions. Several clones were sequenced and a clone containing the predicted sequence was chosen for further use.

The V_{H} fragment was gel purified and cloned into pConG1f0.4. For this the V_{H} fragment was isolated from the pPCR-Script CAM vector after digestion with HindIII and ApaI and gel purification.

The pConG1f0.4 vector was digested with HindIII and ApaI and the vector fragment was isolated from gel, followed by dephosphorylation with Shrimp Alkaline Phosphatase (New England Biolabs) The V_{H} fragment and the pConG1f0.4HindIII-ApaI dephosphorylated fragment were ligated and transformed into competent DH5α-T1^{R} cells (Invitrogen). Eight colonies were checked by colony PCR (using primers pConG1seq1 (P10) and HCseq5 (P11) (FIG. 14) and all colonies were found to contain the correct insert size. A clone was chosen for further study and named pConG1f7D8.

### Example 30

### Construction of pConK7D8: A Vector for Production of the Light Chain of 7D8-IgG1, 7D8-IgG4 and 7D8-HG

The V_{L} coding region of CD20 specific HuMab-7D8 (WO 04/035607) was amplified from a plasmid containing this region using the primers 7D8VLexfor (P7) and 7D8VLexrev (P6) (FIG. 14), introducing suitable restriction sites for cloning into pConKappa0.4 (Lonza Biologics), a mammalian expression vector containing the constant kappa light chain region (allotype km3) of human IgG, and an ideal Kozak sequence.

The PCR product and the pConKappa0.4 vector were digested with HindIII and BsiWI. The vector and V_{L} fragment were purified and the vector was dephosphorylated with Shrimp Alkaline Phosphatase. The V_{L} fragment and the pConKappa0.4HindIII-BsiWI digested vector were ligated and transformed into competent DH5α T1^{R} E. coli. Ten colonies were checked by colony PCR (using primers pConKseq1 (P9) and LCseq3 (P5) (FIG. 14) and 9 colonies were found to contain the correct insert size.

From 4 clones plasmid DNA was isolated and the V_{L} region was sequenced. 3 clones contained the predicted sequence and one clone was chosen for further use and named pConK7D8.

### Example 31

### Construction of pTomG4: A Vector for the Expression of Variable Heavy Chain Regions of Human IgG with the Constant Region of human IgG4

Genomic DNA was isolated from a blood sample of a volunteer and used as a template in a PCR with primers IgG4gene2f (P15) and IgG4gene2r (P14) (FIG. 14), amplifying the complete genomic constant region of the heavy chain of IgG4 and introducing suitable restriction sites for cloning into the mammalian expression vector pEE6.4 (Lonza Biologics). The PCR fragment was purified and cloned into pEE6.4. For this the PCR product was digested with HindIII and EcoRI, followed by heat inactivation of the restriction enzymes. The pEE6.4 vector was digested HindIII and EcoRI, followed by heat inactivation of the restriction enzymes and dephosphorylation of the vector fragment with shrimp alkaline phosphatase, followed by heat inactivation of the phosphatase. The IgG4 fragment and the pEE6.4HindIII/EcoRI dephosphorylated vector were ligated and transformed into competent MACH1-T1^{R} cells (Invitrogen). Three clones were grown in LB and plasmid DNA was isolated from a small culture (1.5 ml). Restriction digestion revealed a pattern consistent with the cloning of the IgG4 fragment in the pEE6.4 vector. Plasmid DNA from two clones was transformed in DH5α-T1^{R} E. coli and plasmid DNA was isolated and the constructs were checked by sequence analysis of the insert and one clone was found to be identical to a genomic IgG4 clone from the Genbank database, apart from some minor differences in introns. SEQ ID No: 13 shows the sequence of the IgG4 region in pTomG4. These differences are presumably either polymorphisms or sequence faults in the Genbank sequence. The plasmid was named pTomG4.

### Example 32

### Construction of pTomG47D8: A Vector for the Production of the Heavy Chain of 7D8-IgG4

Plasmid DNA from pConG1f7D8 was digested with HindIII and ApaI and the V_{H} fragment was gel purified. The pTomG4 vector was digested with HindIII and ApaI and the vector fragment was isolated from gel. The V_{H} fragment and the pTomG4HindIII-ApaI fragment were ligated and transformed into competent DH5α-T1^{R} cells. Four colonies were checked by colony PCR (using primers pConKseq1 (P9) and HCseq11 (P12)) and two were found to contain the correct insert size and the presence of the pTomG4 backbone was confirmed by a digestion with MspI on the colony PCR fragment. One of the clones was chosen for further use. This plasmid was named pTomG47D8.

### Example 33

### Construction of pTomG47D8HG; A Vector for the Expression of the Heavy Chain of 7D8-HG

Site directed mutagenesis was used to destroy the splice donor site of the hinge exon of IgG4 in the pTomG47D8 plasmid. A site-directed mutagenesis reaction was done according to the QuickChange XL site-directed mutagenesis method using primers IgG4S228Pf (P16) and IgG4S228Pr (P17). 24 colonies were screened by colony PCR and XmaI digestion (an extra XmaI site was introduced during mutagenesis) and all colonies appeared to contain the correct nucleotide changes. Two positive colonies were grown overnight, plasmid DNA was isolated and sequenced to confirm that the correct mutation was introduced. Both did contain the correct sequence and one was chosen for further propagation and named pTomG47D8HG. To exclude the introduction of additional mutations during the mutagenesis process, the whole IgG4 coding region of pTomG47D8HG was resequenced and no additional mutations were found. The final vector was named pTomG47D8HG.

### Example 34

### Cloning of the Variable Regions of the Mouse Anti-Betv1 Antibody

Total RNA was prepared from 0.3×10⁵ mouse hybridoma cells (Clone 2H8 from reference (Akkerdaas J H et al., Allergy 50(3), 215-20 (1995)) with the RNeasy kit (Qiagen, Westburg, Leusden, Netherlands) according to the manufacturer's protocol. 5′-RACE-Complementary DNA (cDNA) of RNA was prepared from 112 ng total RNA, using the SMART RACE cDNA Amplification kit (BD Biosciences Clontech, Mountain View, Calif., USA), following the manufacturer's protocol.

The V_{L} and V_{H} regions of the Betv1 antibody were amplified by PCR. For this PfuTurbo® Hotstart DNA polymerase (Stratagene) was used according to the manufacturer's instructions. Each reaction mix contained 200 μM mixed dNTPs (Roche Diagnostics), 12 pmol of the reverse primer (RACEG1 mm1 (P19) for the V_{H} region and RACEKmm1 (P18) for the V_{L} region), 7.2 pmol UPM-Mix (UPM-Mix: 2 μM ShortUPMH3 (P20) and 0.4 μM LongUPMH3 (P21) oligonucleotide (FIG. 14)), 0.6 μl of the 5′RACE cDNA template as described above, and 1.5 unit of PfuTurbo® Hotstart DNA polymerase in PCR reaction buffer (supplied with polymerase) in a total volume of 30 μl.

PCR reactions were carried out with a TGradient Thermocycler 96 (Whatman Biometra) using a 35-cycle program: denaturing at 95° C. for 2 min; 35 cycles of 95° C. for 30 sec, a 55° C. for 30 sec, and 72° C. for 1.5 min; final extension at 72° C. for 10 min.

The reaction products were separated by agarose gel electrophoresis on a 1% TAE agarose gel and stained with ethidium bromide. Bands of the correct size were cut from the gels and the DNA was isolated from the agarose using the QiaexII gel extraction kit (Qiagen).

Gel isolated PCR fragments were A tailed by a 10 min 72° C. incubation with 200 μM dATP and 2.5 units Amplitaq (Perkin Elmer) and purified using minielute columns (Qiagen). A-tailed PCR fragments were cloned into the pGEMTeasy vector (Promega) using the pGEMT easy vector system II kit (Promega), following the manufacturer's protocol. 2 μl of the ligation mixture was transformed into OneShot DH5αT1R competent E. Coli (Invitrogen) and plated on LB/Amp/IPTG/Xgal plates.

Four insert containing, white colonies each for the V_{H} and V_{L} sequences were picked and the inserts were sequenced. The deduced amino acid sequences of the V_{H} and V_{L} of Betv1 are shown as SEQ ID No: 8 and SEQ ID No:12, respectively.

### Example 35

### Construction of pConG1fBetV1: A Vector for the Production of the Heavy Chain of Betv1-IgG1

The V_{H} coding region of mouse anti-BetV1 antibody was amplified by PCR from a plasmid containing this region (example 18) using the primers VHexbetv1for (P4) and VHexbetv1rev (P3), introducing suitable restriction sites for cloning into pConG1f0.4 and an ideal Kozak sequence.

The V_{H} fragment was gel purified and cloned into pConG1f0.4. For this the PCR product and the pConKappa0.4 vector were digested with HindIII and ApaI and purified.

The V_{H} fragment and the pConG1f0.4HindIII-ApaI digested vector were ligated and transformed into competent DH5α-T1^{R} cells.

A clone was selected containing the correct insert size and the correct sequence was confirmed. This plasmid was named pConG1fBetv1.

### Example 36

### Construction of pConKBetv1: A Vector for the Production of the Light Chain of Betv1

The V_{L} coding region mouse anti-BetV1 antibody was amplified from a plasmid containing this region (example 18) using the primers VLexbetv1for (P2) and VLexbetv1 rev (P1), introducing suitable restriction sites for cloning into pConK0.4 and an ideal Kozak sequence.

The PCR product and the pConKappa0.4 vector were digested with HindIII and BsiWI and purified. The V_{L} fragment and the pConKappa0.4HindIII-BsiWI digested vector were ligated and transformed into competent DH5α T1^{R} E. coli.

A clone was selected containing the correct insert size and the sequence was confirmed. This plasmid was named pConKBetv1.

### Example 37

### Construction of pTomG4Betv1: A Vector for the Production of the Heavy Chain of Betv1-IgG4

To construct a vector for expression of Betv1-IgG4, the V_{H} region of BetV1 was cloned in pTomG4.

For this, pTomG4 and pConG1fBetv1 were digested with HindIII and ApaI and the relevant fragments were isolated.

The Betv1 V_{H} fragment and the pTomG4HindIII-ApaI digested vector were ligated and transformed into competent DH5α-T1^{R} cells.

A clone was selected containing the correct insert size and the sequence was confirmed. This plasmid was named pTomG4Betv1.

### Example 38

### Construction of pTomG4Betv1 HG; A Vector for the Production of the Heavy Chain of Betv1-HG

To make a construct for expression of Betv1-HG, the V_{H} region of Betv1 was cloned in pTomG47D8HG, replacing the V_{H} 7D8 region.

For this pTomG47D8HG and pConG1fBetv1 were digested with HindIII and ApaI and the relevant fragments were isolated.

The Betv1 V_{H} fragment and the pTomG47D8HGHindIII-ApaI digested vector fragment were ligated and transformed into competent DH5α-T1^{R} cells.

A clone was selected containing the correct insert size and the sequence was confirmed. This plasmid was named pTomG4Betv1 HG.

### Example 39

### Production of 7D8-IgG1, 7D8-IgG4, 7D8-HG, Betv1-IgG1, Betv1-IgG4, Betv1-HG, 2F8-IgG1, 2F8-IgG4, 2F8-HG, 2F8-Fab, A77-IgG1, A77-IgG4, A77-HG, A77-Fab, cMet-IgG1, cMet-IgG4, cMet-HG, and cMet-Fab by Transient Expression in Hek-293F Cells

Antibodies were produced of all constructs by cotransfecting the relevant heavy and light chain vectors in HEK-293F cells using 293fectin according to the manufacturer's instructions. For 7D8-IgG1, pConG1f7D8 and pConK7D8 were coexpressed. For 7D8-IgG4, pTomG47D8 and pConK7D8 were coexpressed. For 7D8-HG, pTomG47D8HG and pConK7D8 were coexpressed. For Betv1-IgG1, pConG1Betv1 and pConKBetv1 were coexpressed. For Betv1-IgG4, pTomG4Betv1 and pConKBetv1 were coexpressed. For Betv1-HG, pTomG4Betv1HG and pConKBetv1 were coexpressed.

For 2F8-IgG1, pConG1f2F8 and pConK2F8 were coexpressed. For 2F8-IgG4, pTomG42F8 and pConK2F8 were coexpressed. For 2F8-HG, pTomG42F8HG and pConK2F8 were coexpressed. For 2F8-Fab, pEE6.42F8-Fab and pConK2F8 were coexpressed.

For cMet-IgG1, pConG1fcMet and pConKcMet were coexpressed. For cMet-IgG4, pTomG4cMet and pConKcMet were coexpressed. For cMet-HG, pTomG4cMetHG and pConKcMet were coexpressed. For cMet-Fab, pEE6.4cMet-Fab and pConKcMet were coexpressed.

For A77-IgG1, pConG1fA77 and pConKA77 were coexpressed. For A77-IgG4, pTomG4A77 and pConKA77 were coexpressed. For A77-HG, pTomG4A77HG and pConKA77 were coexpressed. For A77-Fab, pEE6.4A77-Fab and pConKA77 were coexpressed.

### Example 40

### Purification of IgG1, IgG4 and IgG4-Hingeless Antibodies

All IgG1, IgG4 and hingeless antibodies were purified. First the supernatants were filtered over 0.20 μM dead-end filter. Then, the supernant was loaded on a 5 ml Protein A column (rProtein A FF, Amersham Bioscience) and eluted with 0.1 M citric acid-NaOH, pH 3. The eluate was immediately neutralized with 2 M Tris-HCl, pH 9 and dialyzed overnight to 12.6 mM sodium phosphate, 140 mM NaCl, pH 7.4 (B. Braun, Oss, The Netherlands). After dialysis samples were sterile filtered over 0.20 μM dead-end filter.

Antibodies were deglycosylated by overnight incubation at 37° C. with 1 unit PNgase F (Roche)/μg antibody, followed by purification on protein A.

Samples were analysed for concentration of IgG by nephelometry and absorbance at 280 nm.

### Example 41

### Purification of Recombinant Fab Antibodies by Metal Affinity Chromatography

Talon beads (Clontech) were used for purification of the A77-Fab, 2F8-Fab and cMet-Fab antibodies.

Before use, the beads were equilibrated with 1× equilibration/wash buffer pH 7.0 (50 mM sodium phosphate and 300 mM NaCl) followed by incubation with the culture supernatant containing the Fab antibody. The beads were washed with 1× equilibration/wash buffer to remove a specific bound proteins and the His-tagged protein was eluted with 1× elution buffer (50 mM sodium phosphate, 300 mM NaCl and 150 mM Imidazole) at pH 5.0. Incubation was done batch wise, whereas washing and elution were done in packed columns using centrifugation (2 minutes at 700 g). The eluted protein was desalted on a PD-10 column by exchanging to PBS. The yield of purified protein was determined by measuring the absorbance at 280 nm using the theoretic absorbance coefficient as calculated from the amino acid sequence. Purified proteins were analyzed by SDS-PAGE, the protein migrated as one band at the expected size.

### Example 42

### Non-Reduced SDS-PAGE Analysis of 7D8-IgG4 and 7D8-HG Antibodies

After purification, the CD20 specific antibodies 7D8-IgG1 (IgG1 anti-CD20) 7D8-IgG4 (IgG4 anti-CD20) and 7D8-HG (hingeless IgG4 anti-CD20) were analysed on non-reducing SDS-PAGE.

The Bis-Tris electrophoresis method used is a modification of the Laemmli method (Laemmli UK, Nature 227, 6801 (1970)), where the samples were run at neutral pH. The SDS-PAGE gels were stained with Coomassie and digitally imaged using the GeneGenius (Synoptics, Cambridge, UK).

As can be seen in FIG. 1, 7D8-IgG1 showed 1 major bind representing the full length tetrameric (2 heavy and two light chains) 7D8 IgG1 molecule. 7D8-IgG4 shows to have besides the major band representing the tetrameric IgG4 molecule a substantial amount of half-molecules (i.e. one heavy band one light chain) as has been described in literature Schuurman J et. al., Mol Immunol 38, 1 (2001); Angal S et al., Mol Immunol 30, 105 (1993); Colcher D et al., Cancer Res 49, 1738 (1989); King D J et al., Biochem J 281(Pt 2), 317 (1992); Petersen J G et al., J Biol Chem 249, 5633 (1974)). The hingeless IgG4 molecule 7D8-HG is shown to be only half-molecules.

### Example 43

### Mass Spectrometry of 7D8-HG

For Mass Spectrometry by Nanospray technique the samples were concentrated and buffer was exchanged to 20 mM sodium phosphate, pH 7.2 using Millipore Microcon YM-30 concentrators. Subsequently, approximately 100 μg IgG was digested for 16 hours at 37° C. with 1 U N-glycosidase F (Roche, cat. no. 1365177) to release the N-linked glycans. Samples were desalted off-line using a C4 micro-trap cartridge and eluted in 30% propanol/5% acetic acid. Molecular weight analysis was performed using nanospray Electrospray-MS using a Q-TOF (Waters, Almere, the Netherlands). The instrument was calibrated using glu-fibrinopeptide. Masslynx 4.0 software was used to deconvolute the multiply-charged data obtained.

A further aliquot of the sample was reduced using dithiothreitol. The products of reduction were desalted off-line using a C4 microtrap and analyzed as described above. MS analysis of 7D8-HG under reducing conditions showed a light chain mass of 23440 dalton which is consistent with the predicted light chain mass of 23440 dalton. No mass of the heavy chain was detected, probably because of precipitation of the heavy chain.

MS analysis under non-reduced conditions showed a predominant mass of 71520 dalton, which correlates well with the predicted mass (71522 dalton) of a half-molecule (combining one heavy and one light chain) missing the hinge. A tiny amount of a product with a mass of 143041 dalton was observed, probably representing a tetrameric molecule with a hingeless heavy chain.

### Example 44

### Mass Spectometry Peptide Mapping of 7D8-HG

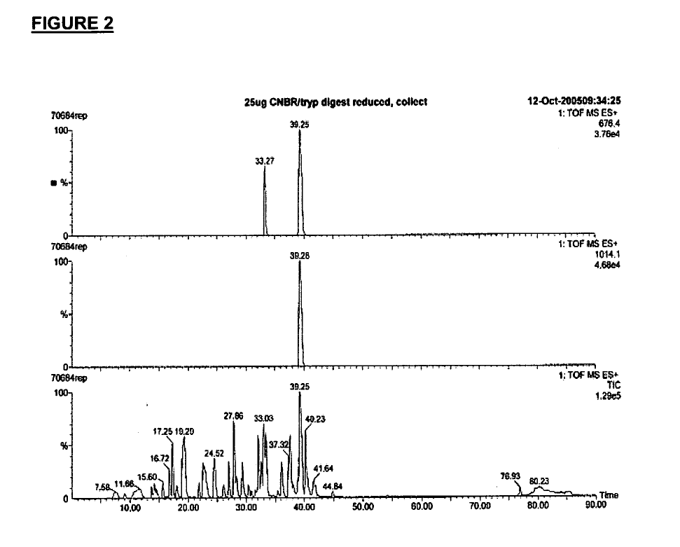

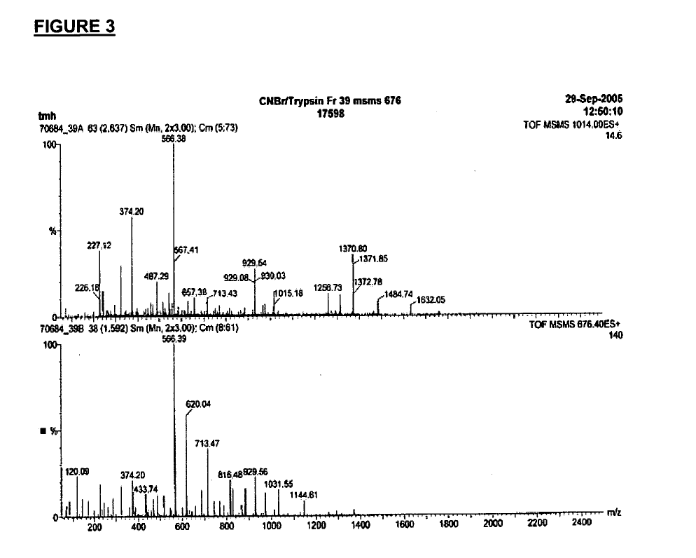

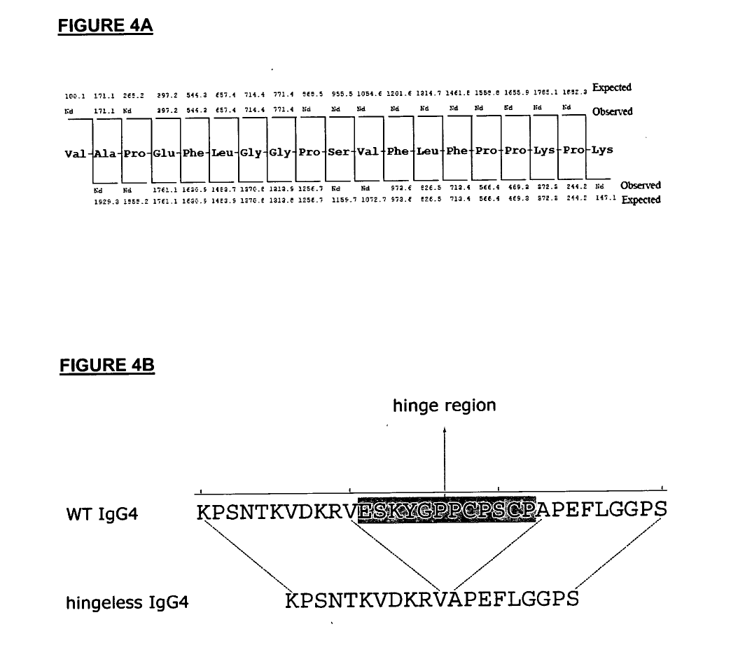

An aliquot (25 μg) of 7D8-HG was digested with CNBr for 5 hours at room temperature. The CNBr digested sample was freeze-dried and then redissolved in 50 mM ammonium bicarbonate buffer adjusted to pH 8.4 with 10% aq. ammonia and digested with TPCK-treated trypsin for 5 hours at 37° C. The products of digestion were lyophilized and reduction was performed on the digested lyophilized sample using a 20 times molar excess of dithiothreitol (DTT) in Tris-acetate buffer at pH 8.5. The products of the reaction were analyzed by on-line LC/ES-MS using a C18 column. Elution was carried out using aqueous formic acid and an acetonitrile gradient. Detection of masses occurred with a LCT Premier Electrospray mass spectrometer, calibrated over the range of m/z 250 to 3000. A tryptic peptide with a mass of 2026.2 Da corresponding to the theoretic mass of the hingeless specific peptide 220 VAPEFLGGPSVFLFPPKPK 238 was detected (FIG. 2). The identity of this peptide was confirmed by nanospray MS and MS/MS (FIGS. 3 and 4). This result shows that the 7D8-HG antibody does not contain a hinge region.

### Example 45

### Molecular Mass Distribution from Sedimentation Velocity by Analytical Ultracentrifuge (AUC) Experiments of 7D8-HG

A 1 mg/ml sample of 7D8-HG in PBS was send to Nanolytics (Dalgow, Germany) for AUC analysis. A dominant population of 7D8-HG sediments with a velocity of 6.7 S (95%) was identified. A distinct aggregate was found at 11.5 S (2%). The rest of the material was found in higher aggregates.

The sedimentation coefficient of the major fraction indicates that 7D8-HG in PBS predominantly occurs as a dimer with a frictional ratio of 1.4.

Apparently 7D8-HG forms a dimer by low affinity non-covalent interactions, presumably in the CH3 region (Saphire, Stanfield et al. 2002). This dimerization process can be inhibited by using HG molecules in the presence of an excess of irrelevant antibodies (see example 54)
Saphire, E. O., R. L. Stanfield, et al. (2002). “Contrasting IgG structures reveal extreme asymmetry and flexibility.” J Mol Biol 319(1): 9-18.

### Example 46

### Functional Analysis of 7D8-IgG1, 7D8-IgG4 and 7D8-HG Antibodies

Binding to the CD20 antigen of these CD20 specific antibodies was examined by flow cytometry. NSO/CD20 transfected cells (50,000 cells/50 μl) were washed in FACS buffer (FB: PBS, 0.05% BSA, 0.02% NaN₃) and incubated in V-bottom 96-well plates with the test antibodies (50 μl at 4° C. for 30 min). After washing, goat F(ab)₂ anti-humanIgG-kappa labeled with PE (Southern Biotechnology, cat No: 2062-09, www.southernbiotech.com) was added to the cells. Cells were washed in FB and cells were collected in FACS tubes in a total volume of 150 μl. Samples were measured and analyzed by use of FACScalibur™ (Becton Dickinson, San Diego, Calif., USA).

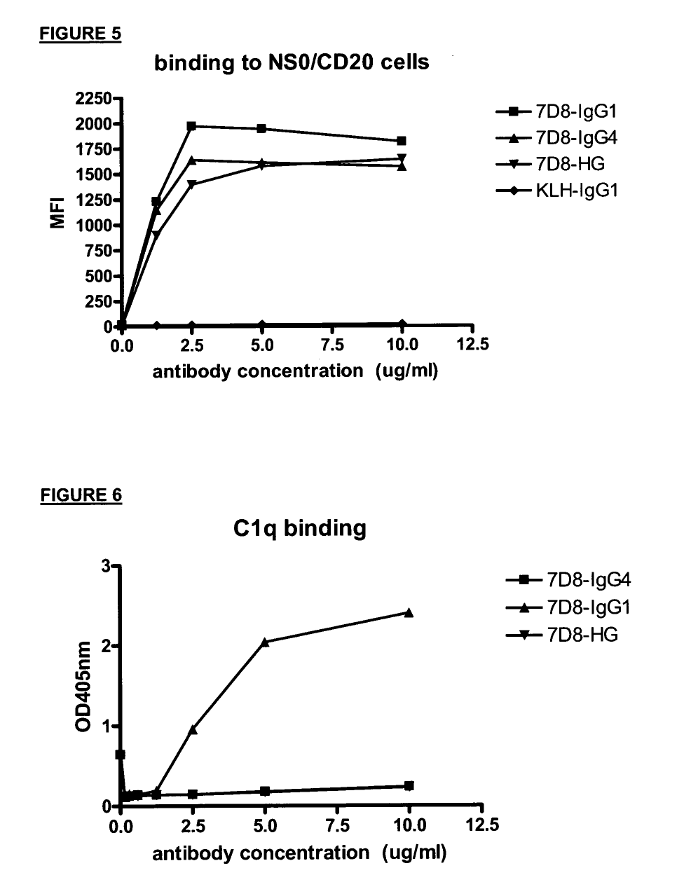

As can be seen in FIG. 5, all three antibodies were antigen specific and showed good binding to CD20.

In order to determine binding of C1q (the first component of the classical complement cascade) to 7D1-IgG1, 7D8-IgG4 and 7D8-HG an ELISA was performed. In short, microtiter ELISA plates (Greiner, Germany) were coated overnight at RT with the test antibodies serially diluted from 10 μg/ml to 0.06 μg/ml in PBS. Plates were emptied and wells were blocked with 200 μl ELISA-diluent per well (0.1 M NaPO₄, 0.1 M NaCl, 0.1% gelatin and 0.05% Tween-20), at RT for 30 minutes. Subsequently, plates were emptied and wells were incubated with 2 μg/ml human C1q (Quidel, lot #900848) in C1q buffer (PBS supplemented with 0.1% w/v gelatine and 0.05% v/v Tween-20, 100 μl/well, 37° C., 1 hour). Plates were washed three times with PBST and wells were incubated with rabbit anti-human C1q (DAKO, A0136), diluted in C1q buffer (100 μl/well, RT, 1 h). After washing the plates (3×) with PBST, wells were incubated with HRP-conjugated swine anti-rabbit IgG-Fc (DAKO, P0300, lot #069) diluted in ELISA diluent (1:2500, 100 μl/well, RT, 1 hour). Thereafter, plates were washed thrice and assays were developed with freshly prepared 1 mg/ml ABTS solution (ABTS: 2,2′-azino-bis[3-ethylbenzthiazoline-6-sulfonic acid]); 2 tablets of 5 mg in 10 ml ABTS buffer, Boehringer Mannheim, Ingelheim, Germany) at RT in the dark for 30 minutes. Absorbance was measured at 405 nm in an ELISA plate reader (Biotek Instruments Inc., Winooski, USA).

As can be seen in FIG. 6, C1q did not bind to both 7D8-IgG4 and 7D8-HG. As a control C1q binding to 7D8-IgG1 was evaluated which showed concentration dependent binding of C1q.

To further investigate the complement properties of the CD20-specific antibodies, the complement-dependent cellular toxicity was examined. After harvesting, Daudi cells (ATCC, www.ATCC.org) were washed trice in PBS and resuspended at 2×10⁶ cells/ml in RPMI 1640, supplemented with 1% (w/v) bovine serum albumin (BSA; Roche, Basel, Switzerland). Then, cells were put in a 96-well round-bottom plate at 1.0×10⁵ cells/well in a volume of 50 μl. The same volume of antibody (highest concentration 10 μg/ml, diluted in RPMI 1640 and 1% BSA) was added to the wells and incubated for 15 minutes at room temperature (RT). Then 25 μl normal human serum (NHS) was added and the cells were incubated at 37° C. for 45 minutes. Heat-inactivated serum (serum AT) is NHS which has been incubated for 10 minutes on 56° C. After incubation for 45 minutes, cells were resuspended transferred to FACS tubes (Greiner). Then, 10 μl propidium iodide (PI; Sigma-Aldrich Chemie B.V.) was added (10 μg/ml solution) to this suspension. Lysis was detected by flow cytometry (FACScalibur™, Becton Dickinson, San Diego, Calif., USA) by measurement of the number of dead cells (PI-positive cells).

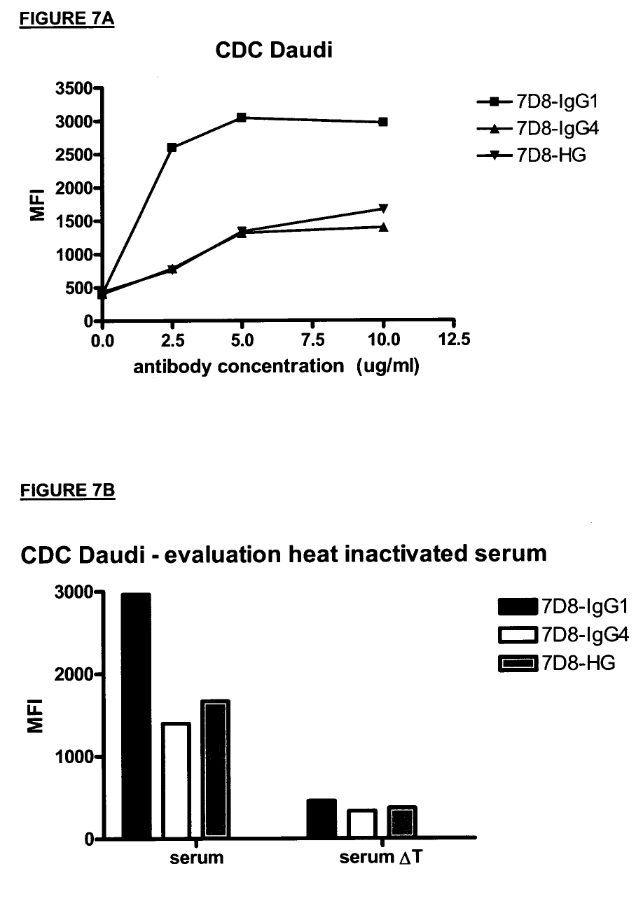

As can be seen in FIG. 7A, 7D8-IgG1 showed good lysis of daudi cells whereas both 7D8-IgG4 and 7D8-HG showed a decreased lysis of Daudi cells.

To evaluate the role of serum, heat-inactivated serum (serum AT) was added to cells incubated with 10 μg antistof. FIG. 7B showed that the induction of lysis was dependent on complement-active serum, addition of heat-inactivated serum resulted in no lysis.

### Example 47

### Non-Reduced SDS-PAGE Analysis of Betv1-HG Antibody

After purification, the Betv1-HG (hingeless IgG4 anti-Bet v1) was analysed on non-reducing SDS-PAGE. The used Bis-Tris electrophoresis method is a modification of the Laemmli method the samples were run at neutral pH. The SDS-PAGE gels were stained with Coomassie and digitally imaged using the GeneGenius (Synoptics, Cambridge, UK).

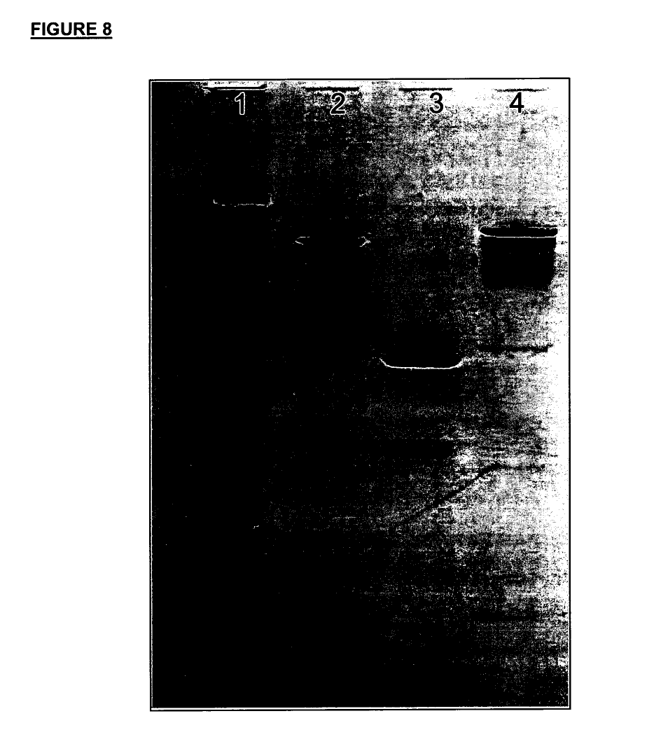

As can be seen in FIG. 8, Betv1-HG showed 1 major bind representing a half-molecule (i.e. one heavy and one light chain).

### Example 48

### Gelfiltration of Betv1-HG Antibody

Betv1-HG was subjected to gelfiltration to investigate whether this mutant would elute as half-molecule or intact dimer. Samples (100 μl) were applied to a Superdex 200 HR 10/30 column (Amersham Biosciences, Uppsala, Sweden), which was connected to a HPLC system (ÄKTA explorer) from Amersham Biosciences, Uppsala, Sweden. The column was first equilibrated in PBS. Fractions of 250 μl were collected, in which Bet v 1 specific IgG was measured using the antigen binding assay. The samples were also followed by measuring the absorption at 214 nm.

To test the antigen binding of the Bet v 1 specific antibodies, a sample of diluted antibody was incubated overnight at room temperature with 0.75 mg Protein-G sepharose (Amersham Biosciences, Uppsala, Sweden) in 750 μl PBS/AT (PBS supplemented with 0.3% BSA, 0.1% Tween-20, 0.05% NaN3) together with 50 μl diluted ¹²⁵I-labelled Bet v 1 or ¹²⁵I-labelled Fel d 1. Bet v 1 was iodinated by the chloramine-T method with carrier free ¹²⁵I (Amersham Biosciences, Uppsala, Sweden) as described in Aalberse et al. (Serological aspects of IgG4 antibodies. 1983. 130:722-726). After washing the Sepharose suspension with PBS-T (PBS supplemented with 0.1% Tween-20), the bound radioactivity was measured. The results were expressed as the amount of radioactivity relative to the amount added.

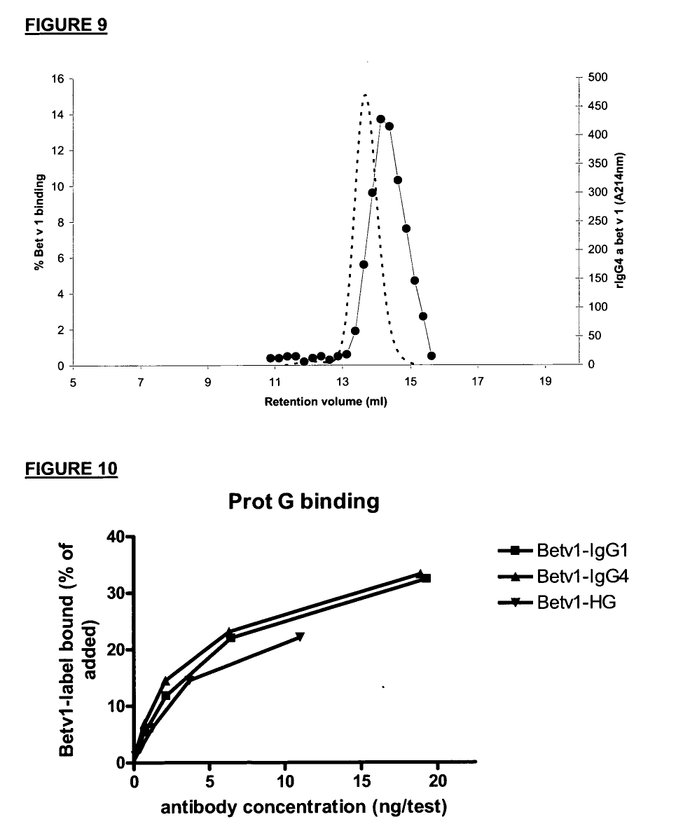

The Bet v 1 binding activity of the hingeless Betv1-HG eluted in one peak, which was more retained than the elution peak of purified Betv1-IgG4 (IgG4 anti Bet v 1) containing an intact hinge (FIG. 9). Calibration of this column using globular proteins showed that the Betv1-HG eluted in fractions corresponding to proteins with a molecular size of ˜70 kD (data not shown). These data support our observations that hingeless IgG4 exists as half-molecules and, in contrast to reported hingeless IgG1 and IgG4 molecules (Silverton E W et al., Proc Natl Acad Sci USA 74, 5140 (1977); Rajan S S et al., Mol Immunol 20, 787 (1983); Horgan C et al., J Immunol 150, 5400 (1993)), does not associate via non-covalent interactions into tetrameric molecules.

### Example 49

### Functional Characterization of Betv1-IgG4 and Betv1-HG Antibodies

Previously was shown that, in contrast to serum-derived antigen specific IgG4, in vitro produced monoclonal IgG4 antibodies are able to crosslink antigen like IgG1 antibodies and are therefore bivalent antibodies (Schuurman J et al., Immunology 97, 693 (1999); Aalberse R C et al., Immunology 105, 9 (2002)). The ability to crosslink antigen of Betv1-IgG1, Betv1-IgG4 and Betv1-HG was determined by a Radio Immuno Assay using Sepharose bound Bet v 1 and ¹²⁵I labelled antigen. Herefore, Birch pollen Sepharose was prepared. Briefly, Birch pollen extract (Allergon, Ängelholm, Sweden) was coupled to CNBr-activated Sepharose 4B (Amersham Biosciences, Uppsala, Sweden) according to the instructions of the manufacturer. Subsequently, the Sepharose was resuspended in PBS supplemented with 0.3% BSA, 0.1% Tween-20, 0.05% NaN₃.

To examine the ability of the antibody to crosslink Sepharose bound antigen to ¹²⁵I labelled antigen, 50 μl of diluted antibody was incubated overnight at room temperature with 750 μl Sepharose in PBS/AT. Next, the Sepharose suspension was washed with PBS-T, after which the suspension was incubated overnight at room temperature with 50 μl diluted ¹²⁵I labelled Bet v1 in a total volume of 750 μl PBS/AT. Finally, the Sepharose was washed with PBS-T and bound radioactivity was measured. The results were expressed as the amount of radioactivity bound relative to the amount of radiolabel added.

As can be seen in FIG. 10, all three antibodies were antigen specific and showed good binding to radiolabelled Betv1.

In FIG. 11 is shown that Betv1-IgG1 and Betv1-IgG4 are able to crosslink Sepharose-bound Bet v 1 to radiolabelled Bet v 1. The IgG1 and IgG4 antibody behave as bivalent antibodies. The Betv1-HG antibody was not able to crosslink the Betv1 antigen and therefore demonstrated monovalent binding.

### Example 50

### Pharmacokinetic Evaluation of an IgG4 Hingeless Mutant Antibody, Compared to Normal IgG1, IgG4 and IgG1 Fragments

Twenty-five SCID mice (C.B-17/IcrCrl-scid-BR, Charles-River) with body weights between 24 and 27 g were used for the experiment. The mice were housed in a barrier unit of the Central Laboratory Animal Facility (Utrecht, The Netherlands) and kept in filter-top cages with water and food provided ad libitum. All experiments were approved by the Utrecht University animal ethics committee.

Monoclonal antibodies were administered intravenously via the tail vein. 50 μl blood samples were collected from the saphenal vein at 1 hour, 4 hours, 24 hours, 3 days, 7 days, 14 days, 21 days and 28 days after administration. Blood was collected into heparin containing vials and centrifuged for 5 minutes at 10,000 g. Plasma was stored at −20° C. for determination of mAb concentrations.

In this experiment the clearance of the hingeless IgG4 variant (7D8-HG, lot 570-003-EP) was compared with that of normal human IgG4 (7D8-IgG4, lot 570-002-EP), a IgG1 variant (7D8-IgG1, lot 793-001-EP), F(ab′)₂ (7D8-G1-F(ab′)₂, lot 815-004-XX) and Fab fragments (7D8-G1-Fab, 815-003-X) of the latter mAb. Each antibody was administered to 5 mice, at a dose of ^{˜}0.1 mg in 200 μl per mouse.

Human IgG concentrations were determined using a sandwich ELISA. Mouse mAb anti-human IgG-kappa clone MH19-1 (#M1272, CLB Sanquin, The Netherlands), coated to 96-well Microlon ELISA plates (Greiner, Germany) at a concentration of 100 ng/well was used as capturing antibody. After blocking plates with PBS supplemented with 2% chicken serum, samples were added, serially diluted in ELISA buffer (PBS supplemented with 0.05% Tween 20 and 2% chicken serum), and incubated on a plate shaker for 1 h at room temperature (RT). Plates were subsequently incubated with peroxidase-labeled F(ab′)₂ fragments of goat anti-human IgG immunoglobulin (#109-035-097, Jackson, West Grace, Pa.) and developed with 2,2′-azino-bis(3-ethylbenzthiazoline-6-sulfonic acid) (ABTS; Roche, Mannheim, Germany). Absorbance was measured in a microplate reader (Biotek, Winooski, Vt.) at 405 nm.

SCID mice were chosen because they have low plasma IgG concentrations and therefore relatively slow clearance of IgG. This provides a PK model that is very sensitive for detecting accelerated clearance due to diminished binding of the Fcγ-part to the neonatal Fc receptor (FcRn).

Pharmacokinetic analysis was done by determining the area under the curve (AUC) from the concentration-time curves, with tailcorrection. The plasma clearance rate was calculated as Dose/AUC (ml/day). Statistical testing was performed using GraphPad PRISM vs. 4 (Graphpad Software).

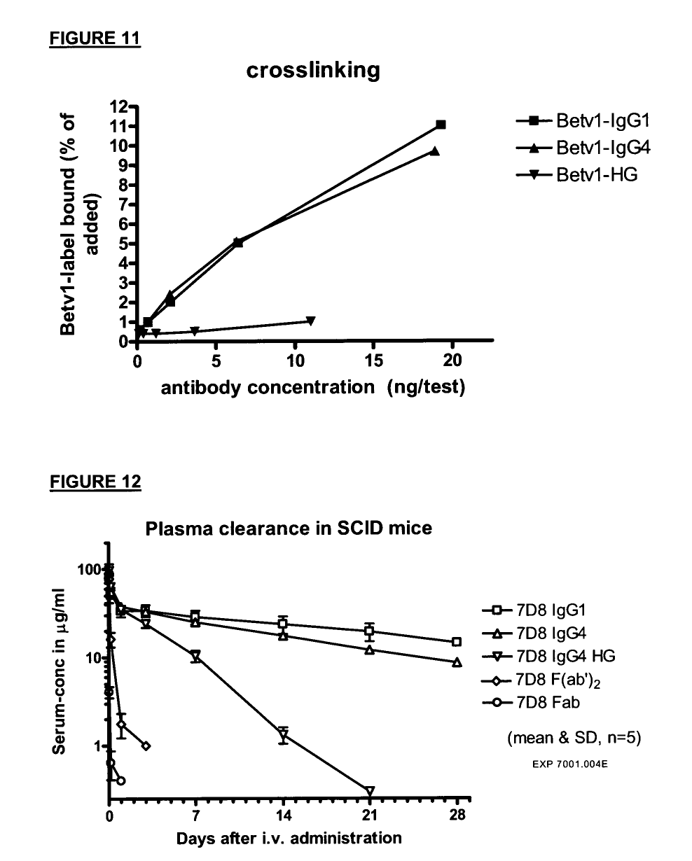

FIG. 12 shows a semilogarithmic plot of the concentrations in time. The initial plasma concentrations were in the same order for all intact mAbs 85-105 ug/ml, including the hingeless variant. These initial concentrations correspond to a central distribution volume of about 1 ml, which is consistent with distribution into the plasma compartment of the mice. For the F(ab′)2 and Fab fragments lower initial concentrations were observed, 75 and 4 ug/ml, respectively. For the Fab fragments this is likely due to rapid extravascular distribution within the first hour after administration.

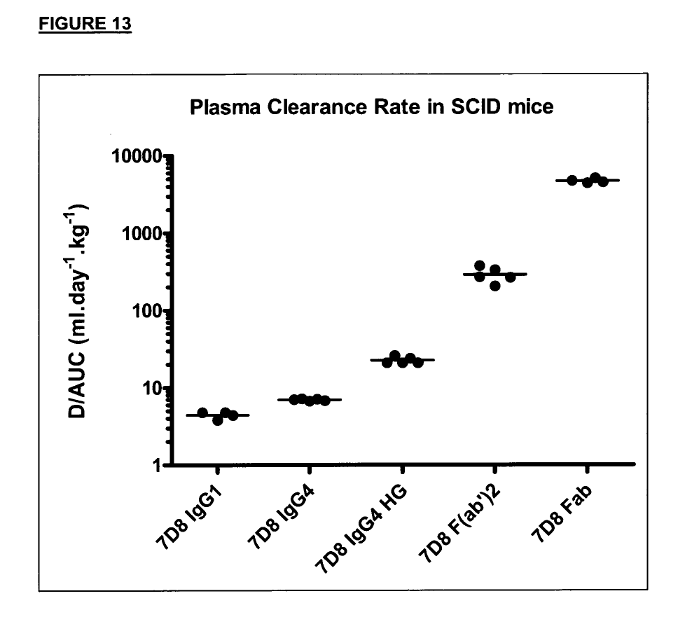

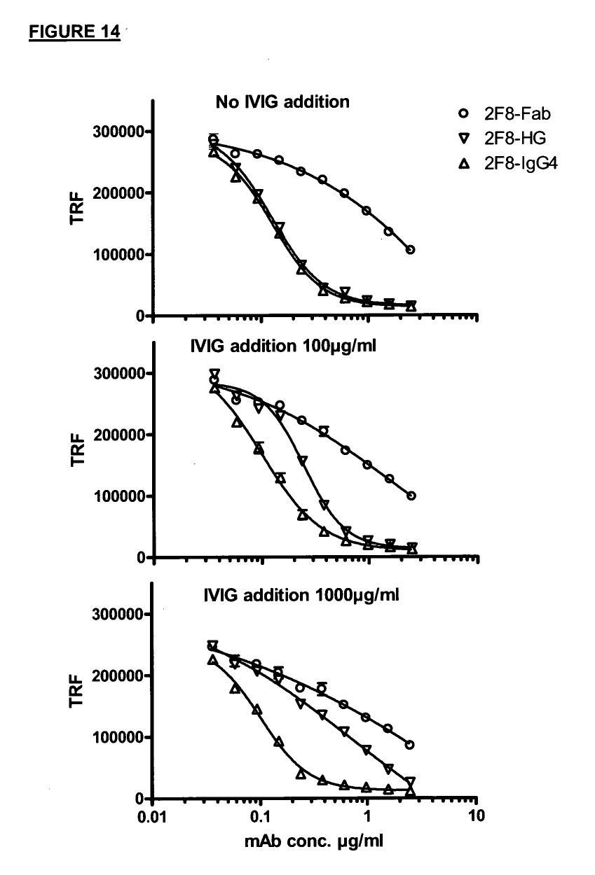

FIG. 13 shows the clearance rates calculated for the individual mice. The clearance rate of the hingeless variant was 3 to 4 times higher than that of normal IgG1 and IgG4. However, it was more than 10 times slower than that of F(ab′)2 fragments and more than 200 times slower than the clearance of Fab fragments.

### Example 51

### Pharmacokinetic Evaluation of an IgG4 Hingeless Mutant Antibody Compared to Normal IgG4 and IgG1 F(ab)2 Fragments in Immune-Competent Mice

Twelve 8-week old Balb/c mice (Balb/CAnNCrl, Charles-River) were used for the experiment. The mice were housed in a barrier unit of the Central Laboratory Animal Facility (Utrecht, The Netherlands) and kept under sterile conditions in filter-top cages with water and food provided ad libitum. All experiments were approved by the Utrecht University animal ethics committee.

Monoclonal antibodies were administered intravenously via the tail vein. 50 μl blood samples were collected from the saphenal vein at 1 hour, 4 hours, 24 hours, 3 days, 7 days, and 10 days after administration. Blood was collected into heparin containing vials and centrifuged for 5 minutes at 10,000 g. Plasma was stored at −20° C. for determination of mAb concentrations.

In this experiment the plasma clearance rate of the hingeless IgG4 variant (7D8-HG, lot 570-003-EP) was compared with that of normal human IgG4 (7D8-IgG4, lot 570-002-EP), a F(ab′)₂ fragments from 7D8 IgG1 (7D8-G1-F(ab′)₂, lot 815-004-XX). Each antibody was administered to 4 mice, at a dose of 0.1 mg in 200 μl per mouse, corresponding to a dose of 4 mg per kg of body weight.

Human IgG plasma concentrations were determined using a sandwich ELISA. Mouse mAb anti-human IgG-kappa clone MH19-1 (#M1272, CLB Sanquin, The Netherlands), coated to 96-well Microlon ELISA plates (Greiner, Germany) at a concentration of 100 ng/well was used as capturing antibody. After blocking plates with PBS supplemented with 2% chicken serum, samples were added, serially diluted in ELISA buffer (PBS supplemented with 0.05% Tween 20 and 2% chicken serum), and incubated on a plate shaker for 1 h at room temperature (RT). After washing, the plates were subsequently incubated with peroxidase-labeled F(ab′)₂ fragments of goat anti-human IgG immunoglobulin (#109-035-097, Jackson, West Grace, Pa.) and developed with 2,2′-azino-bis(3-ethylbenzthiazoline-6-sulfonic acid) (ABTS; Roche, Mannheim, Germany). Absorbance was measured in a microplate reader (Biotek, Winooski, Vt.) at 405 nm.

Balb/c mice were chosen because they have normal IgG production and therefore faster clearance of IgG than SCID mice. This provides a mouse model in which the administered antibodies have to compete with endogenous mouse IgG for binding to the neonatal Fc receptor (FcRn).

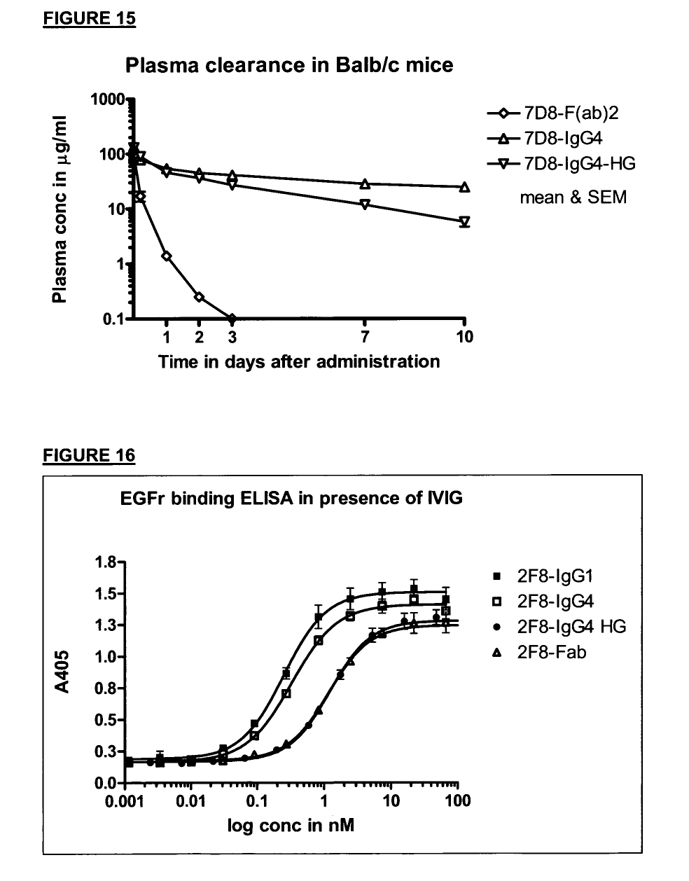

FIG. 15 shows a semilogarithmic plot of the concentrations in time. The initial plasma concentrations were all in the order of 100 μg/ml, which is consistent with an initial distribution into the plasma compartment of the mice. The clearance of the hingeless IgG4 variant was only slightly faster than that of normal IgG4. Importantly, the clearance of the hingeless variant was much slower than that of F(ab′)₂ fragments, which have a comparable molecular size.

This experiment indicates that the Fc-part has a favorable effect on the plasma residence time in mice having a normal immune system and provides an indication of a functional interaction with the neonatal Fc receptor (FcRn) also in the presence of endogenous IgG.

### Example 52

### Pharmacokinetic Evaluation of an IgG4 Hingeless Mutant Antibody in Human IgG-Supplemented SCID Mice

Sixteen SCID mice (C.B-17/IcrCrl-scid-BR, Charles-River) with body weights between 18 and 22 g were used for the experiment. The mice were housed in a barrier unit of the Central Laboratory Animal Facility (Utrecht, The Netherlands) and kept under sterile conditions in filter-top cages with water and food provided ad libitum. All experiments were approved by the Utrecht University animal ethics committee.

Immunodeficient SCID mice were chosen for studying the pharmacokinetics of the hingeless IgG4 variant, because these mice do not develop antibody responses to human proteins which may affect clearance studies with durations of more than one week. These IgG-deficient mice were supplemented with a high dose of intravenous immunoglobulin (human multidonor polyclonal IgG) to study the clearance of hingeless IgG4 mutant in the presence of human IgG at physiologically relevant concentrations. This provides a mouse model which better represents the conditions in humans, because 1) association of hingeless IgG4 into a bivalent form is prevented by the presence of IVIG, and 2) hingeless IgG4 has to compete with other IgG for binding to the neonatal Fc receptor (FcRn)¹. Binding to FcRn protects IgG from intracellular degradation after endocytosis and is responsible for its long plasma half-life. ¹ Bazin R, et al. Use of hu-IgG-SCID mice to evaluate the in vivo stability of human monoclonal IgG antibodies. J Immunol Methods. 1994; 172: 209-17.

In this model the plasma clearance was studied of variants from the human CD20 specific human mAb clone 7D8. The clearance rate of the hingeless IgG4 variant (7D8-HG, lot 992-001-EP) was compared with that of normal human IgG4 (7D8-IgG4, lot 992-002-EP), of F(ab′)₂ fragments from 7D8 IgG1 (7D8-F(ab′)₂, lot 892-020-XX). In addition, a preparation of the hingeless variant tested that was enzymatically deglycosylated (TH3001-7D8-HG deglyc, lot 991-004-EP). Each antibody was administered to 4 mice via the tail vein, at a dose of 0.1 mg in 200 μl, corresponding to a dose of about 5 mg per kg of body weight. The monoclonal antibodies were administered in a 1:1 mixture with Intravenous Immunoglobulin (60 mg/ml, Sanquin, The Netherlands, JFK108ST, charge#04H04H443A). The total injected volume was 400 μl/mouse, giving an IVIG dose of 12.5 mg per mouse.

Fifty μl blood samples were collected from the saphenal vein at 15 minutes, 5 hours, 24 hours, 2 days, 3 days, 7 days, and 10 days after administration. Blood was collected into heparin containing vials and centrifuged for 10 minutes at 14,000 g. Plasma was stored at −20° C. for determination of mAb concentrations. Plasma concentrations of the 7D8 variants were determined using a sandwich ELISA. A mouse mAb anti-7D8-idiotype antibody (clone 2F2 SAB 1.1 (LD2), lot 0347-028-EP) was used as capturing antibody. After blocking plates with PBS supplemented with 0.05% Tween and 2% chicken serum, samples were added, serially diluted in ELISA buffer (PBS supplemented with 0.05% Tween 20 and 2% chicken serum), and incubated on a plate shaker for 2 h at room temperature (RT). The infused antibodies were used as reference. After washing, the plates were subsequently incubated with peroxidase-labeled goat anti-human F(ab′)₂ specific (109-035-097, Jackson Immunoresearch, West Grace, Pa.) and developed with 2,2′-azino-bis(3-ethylbenzthiazoline-6-sulfonic acid) (ABTS; Roche, Mannheim, Germany). Absorbance was measured in a microplate reader (Biotek, Winooski, Vt.) at 405 nm. Total human IgG plasma concentrations were determined using a similar ELISA. Mouse mAb anti-human IgG-kappa clone MH16 (#M1268, CLB Sanquin, The Netherlands) was used as capturing antibody. Peroxidase-labeled goat anti-human IgG immunoglobulin (#109-035-098, Jackson, West Grace, Pa.) was used for detection.

Pharmacokinetic analysis was done by determining the area under the curve (AUC) from the concentration-time curves, with tail correction. The plasma clearance rate was calculated as Dose/AUC (ml/day). Statistical testing was performed using Graph Pad PRISM vs. 4 (Graphpad Software).

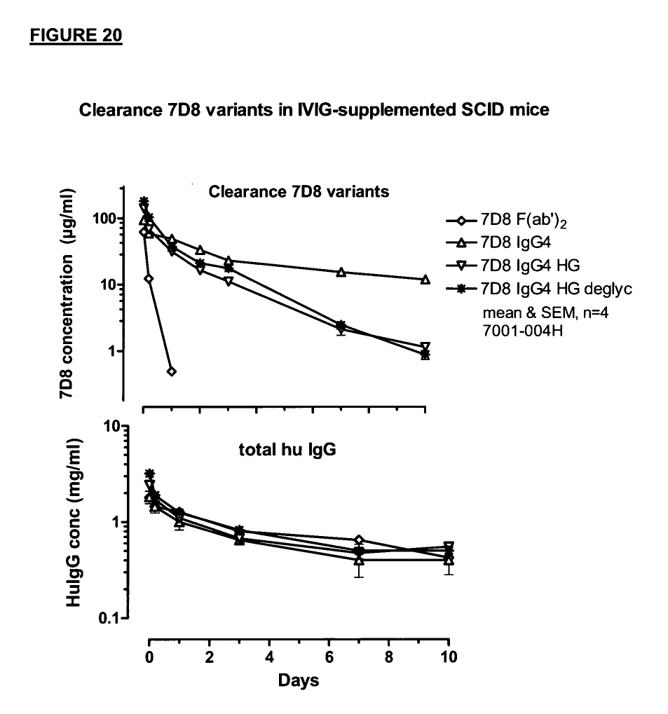

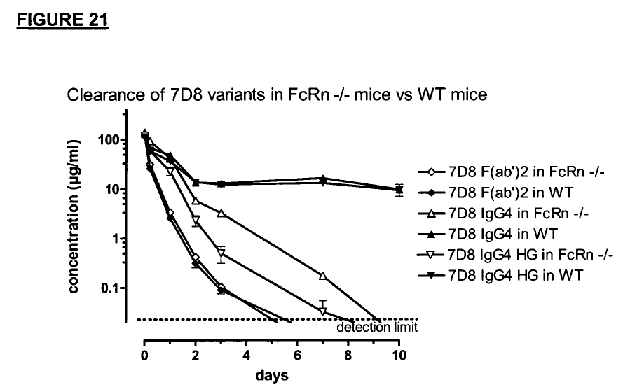

FIG. 20 shows in the upper panel semi-logarithmic plots of the concentrations of the mAb 7D8 variants in time and in the lower panel the total human IgG concentrations. The initial total human IgG concentrations were on average 2.3 mg/ml and declined to 0.47 mg/ml after 10 days. The initial plasma concentrations of 7D8 IgG4 and IgG4 HG variants were in the range of 94 to 180 μg/ml, which is consistent with an initial distribution into the plasma compartment of the mice. For the F(ab′)₂ fragments the initial concentrations were somewhat lower, on average 62 μg/ml. The upper panel makes clear that the clearance of the hingeless variant, including the deglycosylated preparation, is somewhat faster than that of intact IgG4, but much slower than that of F(ab′)₂ fragments. The table 1 below shows the clearance rates calculated from the concentration-time curves. The clearance rate of the hingeless variant was 2 to 3 times higher than that of normal IgG4. However, it was almost 10 times slower than that of F(ab′)2 fragments. Importantly, deglycosylation had no significant effect on the rate of clearance of the hingeless IgG4 variant.

TABLE 1

| | PLASMA CLEARANCE RATE | | | |
|---|---|---|---|---|
| | (D/AUC) in ml/day per kg | | | |
| | IgG1 | | | IgG4 HG |
| | F(ab′)2 | IgG4 | IgG4 HG | deglyc |
| Mean | 380 | 14 | 39 | 29 |
| Lower 95% CI of mean | 346 | 12 | 25 | 19 |
| Upper 95% CI of mean | 415 | 17 | 53 | 38 |
| Number of values | 4 | 4 | 4 | 4 |

Thus, also in the presence of human IgG in physiologically relevant concentrations the clearance of the hingeless variant is much slower than that of F(ab′)2 fragments, which have a comparable molecular size. This experiment demonstrates that, also in the presence of competing human IgG at physiologically relevant concentrations, the hingeless IgG4 variant is capable of functional interaction with the neonatal Fc receptor (FcRn). Furthermore, this experiment indicates that the glycosylation of the hingeless IgG4 variant does not affect plasma clearance and that non-glycosylated hingeless IgG4 has a similar half-life in vivo as the fully glycosylated from.

### Example 53

### Pharmacokinetic Evaluation of an IgG4 Hingeless Mutant Antibody Compared to Normal IgG4 and IgG1 F(ab)₂ Fragments in FcRn −/− Mice

This experiment was performed to investigate whether the IgG4 hingeless mutant is capable of interacting with the neonatal Fc receptor (FcRn), which is responsible for the long plasma half-life of IgG by protecting IgG from intracellular degradation after endocytosis. B2M knockout mice were used in this experiment because they do not express FcRn.

Twelve female C57Bl/6 B2M knockout mice (Taconic model B2MN12-M, referred to as FcRn −/− mice), and twelve female C57Bl/6 wild type control mice (Taconic, model nr. B6, referred to as WT mice) were used for the experiment. The mice were housed in a barrier unit of the Central Laboratory Animal Facility (Utrecht, The Netherlands) and kept in filter-top cages with water and food provided ad libitum. All experiments were approved by the Utrecht University animal ethics committee.

The plasma clearance was studied of variants from the human CD20 specific human mAb clone 7D8. The clearance rate of the hingeless IgG4 variant (7D8-HG, lot 992-001-EP) was compared with that of normal human IgG4 (7D8-IgG4, lot 992-002-EP), F(ab′)₂ fragments from 7D8-IgG1 (7D8-G1-F(ab′)₂, lot 892-020-XX).

Monoclonal antibodies were administered intravenously via the tail vein. Each antibody was administered to 4 mice at a dose of ^{˜}0.1 mg in 200 μl per mouse, corresponding to a dose of 5 mg per kg of body weight. Fifty μl blood samples were collected from the saphenal vein at 10 minutes, 5 hours, 24 hours, 2 days, 3 days, 7 days, and 10 days after administration. Blood was collected into heparin containing vials and centrifuged for 10 minutes at 14,000 g. Plasma was stored at −20° C. for determination of mAb concentrations. Human IgG plasma concentrations were determined using a sandwich ELISA in which mouse mAb anti-human IgG-kappa clone MH19-1 (#M1272, CLB Sanquin, The Netherlands), coated to 96-well Microlon ELISA plates (Greiner, Germany) at 100 ng/well was used as capturing antibody. After blocking plates with ELISA buffer (PBS supplemented with 0.05% Tween and 2% chicken serum), samples were added, serially diluted in ELISA buffer. Serial dilutions of the corresponding infused antibody preparations were used as reference. After incubation and washing, the plates were incubated with peroxidase-labeled AffiniPure Goat Anti-Human IgG, F(ab′)₂ Fragment Specific (#109-035-097, Jackson Immunoresearch, West Grace, Pa.) and developed with 2,2′-azino-bis(3-ethylbenzthiazoline-6-sulfonic acid) (ABTS; Roche, Mannheim, Germany). Absorbance was measured in a microplate reader (Biotek, Winooski, Vt.) at 405 nm. Pharmacokinetic analysis was done by determining the area under the curve (AUC) from the concentration-time curves, with tail correction. The plasma clearance rate was calculated as Dose/AUC (ml/day). Statistical analysis was performed using GraphPad PRISM vs. 4 (Graphpad Software).

FIG. 21 shows a semi-logarithmic plot of the concentrations in time. The initial plasma concentrations were all in the order of 100 μg/ml, which is consistent with an initial distribution in the plasma compartment of the mice. The table 2 below shows the plasma clearance rates calculated from the concentration-time curves of individual mice.

TABLE 2

| PLASMA CLEARANCE RATE | F(ab′)2 | F(ab′)2 | IgG4 | IgG4 | IgG4 HG | IgG4 HG |
|---|---|---|---|---|---|---|
| ml/day per kg | WT | FcRn−/− | WT | FcRn−/− | WT | FcRn−/− |
| Mean | 183 | 159 | 12 | 45 | 15 | 83 |
| Std. Deviation | 19 | 19 | 10 | 3 | 4 | 29 |
| Number of values | 4 | 4 | 4 | 4 | 4 | 4 |

| | | | |
|---|---|---|---|
| Significance difference: Pvalue | 0.1265 | 0.0009 | 0.0033 |
| (t-test) | ns | *** | ** |

For F(ab′)₂ fragments no significant differences were observed between wild type (WT) and knockout (FcRn−/−) mice. In contrast, for IgG4 and the hingeless IgG4 variant the clearance rates were 3 to 5 times slower in the WT mice compared to that in FcRn −/− mice. This experiment shows that the presence of FcRn has a favorable effect on the plasma residence time of hingeless IgG4. Therefore, it provides evidence that hingeless IgG4 is capable having a functional interaction with FcRn in vivo, which explains its favorable plasma half-life.

### Example 54

### Functional Analysis of 2F8-HG anti-EGFr mAb

MAb 2F8 is a human IgG1 monoclonal antibody (mAb) against human Epidermal Growth Factor receptor (EGFr) which is capable to inhibit EGFr signalling by blocking binding of ligands. From this mAb an IgG4 variant, 2F8-IgG4, was made and also a hingeless variant, 2F8-HG.

In the present example, we compared the potency of 2F8-HG with that of 2F8-IgG1 and 2F8-Fab fragments to inhibit ligand-induced EGFr phosphorylation in cells in vitro. This was done both with and without addition of Intravenous Immunoglobulin (IVIG), a polyclonal human IgG preparation, containing all IgG subclasses.

Inhibition of EGFr phosphorylation was measured in a two-step assay using the epidermoid cell line, A431 (ATCC, American Type Culture Collection, Manassas, USA). The cells were cultured overnight in 96-wells plates in serum-free medium containing 0.5% human albumin (human albumin 20%, Sanquin, the Netherlands). Next, mAb were added in serial dilution, with or without IVIG (Immunoglobuline I.V., Sanquin) at a fixed final concentration of either 100 or 1000 μg/ml. After 60 minutes incubation at 37° C., 50 ng/ml recombinant human EGF (Biosource) was added to induce activation of non-blocked EGFr. Following an additional 30 minutes incubation, cells were solubilized with lysis buffer (Cell Signaling Technology, Beverly, Mass.), and the lysates were transferred to ELISA plates coated with 1 μg/ml of mouse anti-EGF-R antibodies (mAb EGFR1, BD Pharmingen, San Diego, Calif.). After 2 hours incubation at RT, the plates were washed and binding of phosphorylated EGF-R was detected using a europium-labelled mouse mAb, specific for phosphorylated tyrosines (mAb Eu-N1 P-Tyr-100, PerkinElmer). Finally, DELFIA enhancement solution was added, and time-resolved fluorescence was measured by exciting at 315 nm and measuring emission at 615 nm on an EnVision plate reader (PerkinElmer). Sigmoidal dose-response curves were calculated using non-linear regression (GraphPad Prism 4).

As can be seen in the upper panel of FIG. 14, 2F8-HG was equally effective as 2F8-IgG1 in inhibiting phosphorylation when culture medium was used without addition IVIG. Both mAb were more potent than 2F8-Fab fragments, which bind monovalently to EGFr. The middle and lower panels of FIG. 14 show that addition of IVIG had negligible effect on 2F8-IgG4 and 2F8-Fab. However, it markedly right-shifted the dose-response curve of 2F8-HG, indicating a change in binding characteristics, which is consistent with the idea that under certain conditions 2F8-HG may behave as a bivalent antibody, but dissociates into a monovalent form in the presence of polyclonal human IgG.

### Example 55

### Proof of Principle: IgG4 Hingeless Against CD89 (CD89-HG) Inhibits IgE-Mediated Asthma in a Mouse Model

Pasquier et al. (Pasquier, B et al., Immunity 22, 31 (2005)) showed that FcαRI (CD89 (Monteiro R C et al., Annu Rev Immunol 21, 177 (2003)) has both an anti- and proinflammatory role. Aggregation of FcαRI leads to cell activation by recruitment of Syk and aborting SHP-1 binding. A monomeric interaction with FcαRI inhibits the activating response: SHP-1 is being recruited and impairment of Syk, LAT and ERK phosphorylation occurs.

Fab fragments of an anti-CD89 antibody (clone A77) could inhibit IgG-mediated phagocytosis using human monocytes. Furthermore, IgE-mediated responses in vitro using FcαRI transfected RBL-2H3 cells and in vivo in an IgE-mediated asthma model were inhibited by Fab fragments of this anti-CD89 antibody. In this animal model, FcαRI-transgenic mice (Launay P et al., J Exp Med 191, 1999 (2000)) were sensitized with TNP-OVA. Mice challenged intranasally with IgE-TNP-OVA immune complexes in the presence of A77 Fab-fragments showed reduced bronchial reactivity to methacholine whereas and irrelevant Fab-fragment could reduce the bronchial hyperreactivity.

Proof on principle in vitro of an antigen specific, non-crosslinking, monovalent, non-activating antibody is obtained in the following experiment. Adherent PBMC are incubated with 10 μg/ml A77-HG (IgG4 hingeless) preincubated 24 h with or without irrelevant IgG4 (Genmab BV) or incubated with irrelevant HG antibody for 30 min at 37° C., washed, and incubated at 37° C. for 30 min with Texas-red-conjugated E. coli (50 bacteria/cell) (Molecular Probes, Eugene, Oreg.) opsonized or not with polyclonal rabbit anti-E. coli IgG antibodies according to the manufacturer's instructions. Slides are mounted and examined with a confocal laser microscope. The PBMC receiving opsonized E. coli and A77-HG (pre-incubated with irrelevant IgG4) show reduced phagocytosis of E. coli when compared to PMBC receiving opsonized E. coli and control-HG antibody.

FcαRI-transgenic mice are sensitized with TNP-OVA as described (Pasquier B et al., Immunity 22, 31 (2005)); or alternatively with OVA as described by Deurloo et al. (Deurloo D T et al., Clin Exp Allergy 33, 1297 (2003)). Human FcαRI transgenic mice and littermate controls are immunized twice on day 0 and day 7 intraperitonally with TNP-OVA or OVA (Sigma) in aluminium hydroxide. Mice are challenged intranasally for a few consecutive days with either TNP-OVA complexed with 20 μg anti-DNP-IgE (Zuberi, R I et al., J Immunol 164, 2667 (2000)) or OVA aerosol (Deurloo D T et al., Clin Exp Allergy 33, 1297 (2003)) in the presence of A77-HG (IgG₄ hingeless) or an irrelevant hingeless antibody (control-HG). The mice receive 50 μg A77-HG or control-HG intraperitoneally twice, once during the challenge period and once with the last intranasal challenge. Twelve hours after the final intranasal challenge, the mice are placed in a whole-body plethysmograph chamber (BUXCO Electronics, Sharon Conn., USA), and 300 mM methacholine delivered. Airway resistance is measured after exposure to methacholine. Immunohistological evaluation is performed on lung sections after euthanizing the mice.

The mice receiving A77-HG show a reduced hyper reactivity when compared to the mice receiving the control-HG antibody.

This indicates that a hingeless IgG₄ molecule is non-crosslinking, monovalent and non-activating and therefore useful for therapeutic purposes where such inert antibody may be favourable such as in the inhibition of inflammatory reactions through FcαRI.

### Example 56

### Proof of Concept Study with Hingeless IgG4 cMet (cMet-HG)

The receptor tyrosine kinase c-Met is prominently expressed on a wide variety of epithelial cells. During embryogenesis, cMet and Hepatocyte Growth factor/Scatter factor (HGF/SF) are involved in tissue-specific differentiation, leading to a proper organization of epithelial cells, muscle endothelium, and the nervous and hematopoietic systems. Abnormal cMet signalling has been implicated in tumorogenesis, particularly in the development of invasive and metastatic tumors. As a consequence of enhanced cMet activity, tumor cells may increase their growth rate and become resistant to apoptosis, resulting in a growth and/or survival advantage. Furthermore, cMet activation may lead to cytoskeletal reorganization and integrin activation, as well as to activation of proteolytic systems involved in extracellular matrix degradation, resulting in an increased invasive and metastatic capacity. Inhibition of HGF/SF-cMet signaling, therefore, represents an important therapeutic avenue for the treatment of malignant tumors.

Kong-Beltran et al. in Cancer Cell (2004 volume 6, pages 75-84) raised an antibody (5D5) to the extracellular domain of cMet and inhibited HGF binding. The Fab fragment of anti-Met 5D5 was shown to inhibit HGF-driven cMet phosphorylation, cell motility, migration and tumor growth. They speculate that anti-cMet-5D5-Fab block receptor dimerization by steric hindering.

MAb C6 is a human IgG1 monoclonal antibody (mAb) against human cMet which is capable of binding with high affinity to H441 cells, activate cMet phosphorylation, induce scattering of DU-145 and block HGF binding to cMet in ELISA. From this mAb a Fab fragment (cMet-Fab), an IgG4 variant (cMet-IgG4), and also a hingeless variant was made (cMet-HG).

In a proof-of-concept study with hingeless IgG4 against cMet (cMet-HG) this monovalent antibody inhibited HGF binding, receptor dimerization/activation, cell scattering, and downstream signalling. This experiment was performed both with and without addition of Intravenous Immunoglobulin (IVIG), a polyclonal human IgG preparation, containing all IgG subclasses and with and without rHGF.

DU-145 Scatter Assay

DU-145 (humane prostate carcinoma cell line, ATCC HTB-81) cells were cultured in DMEM+ (containing 500 ml MEM Dulbecco (DMEM-Medium, glucose 4.5 g/ml with NaHCO3, without glutamine, Sigma, D-6546), 50 ml Cosmic Calf Serum (Hyclone SH30087.03), 5 ml of 200 mM/L L-glutamine (Bio Whittalker, BE17-605F), 5 ml sodium pyruvate (Bio Whittaker BE13-115E), 5 ml penicillin/streptamicin (Bio Whittaker, DE17-603E)) and were growing adherent clustered cells. Upon addition of rhHGF (Sigma, H-1404), migration of the cells was induced, which leads to singularized cells. This process was called scattering. Induction or inhibition of scattering was observed by microscopy.

Day 1: cMet, cMet-HG, cMet-Fab, cMet-IgG4 (30/3.0/0.3/0.03 μg/ml), were incubated over night with and without addition of IVIG, 6 mg/ml. DU145 cells were seeded (adherent cells out of T75-culture flask) cell culture supernatant was removed and cells were washed 1 time with 10 ml PBS 2 ml Trypsine/EDTA was added (37° C.) and cells were incubated at 37° C. for 1-2 min. The cells were removed from the surface of the culture flask by tapping and the Trypsine/EDTA reaction was stopped with stored culture supernatant. The cells were counted and a suspension was prepared of 1′10⁴ cells/ml in fresh culture medium and 50 μl/well was plated into 96-well plate (Sterile flat bottom Costar, 3596) (final density 1000 cells/well). Cells were cultured for 15-24 h at 37° C. and 5% CO₂ in an incubator.

Day 2: Medium was replaced by fresh medium, 40 μl/well. 40 ul of the preincubated antibody was added to the cells and cells were incubated at 37° C. in an incubator for 60 min, after which 40 μl/well medium or 60 ng/ml rh-HGF was added. (Final concentrations were: 10/1.0/0.1/0.01 μg/ml Ab, 2 mg/ml IVIG, 20 ng/ml HGF). Cells were incubated for at least 24 h.

Day 3 and 4: Scattering was observed double-blinded by microscope after 24 h or after 48 h. Morphological characteristics of scattering: cells detach from the surface, show spindle shaped forms (migrate), and most were single cells not in clusters.

Ranking of rh-HGF induced scatter inhibition by antibodies:

3 cells were maximal scattering
2 small inhibition of scattering
1 inhibition of scattering
0 no scattering

In this experiment C6-HG pre-incubated with IVIG significantly blocked the HGF induced scattering.

Phosphorylation of the cMet Receptor

A549 cells were cultured in Ham's F12 medium and cMet was not phosphorylated under normal culture conditions. Upon activation by HGF, the cMet receptor becomes phosphorylated. By applying cMet blocking cMet-Fab or cMet-HG with pre-incubation of IVIG the HGF mediated phosphorylation of the receptor was inhibited.

Day 1: cMet-IgG1, cMet-HG (12.5 μg/ml), were incubated over night with and without addition of IVIG, 2.5 mg/ml. A549 cells (1*10⁶/well) were cultured in a 6 well plate.

Day 2: The culture medium, (containing 500 ml Ham's F12 (Bio Whittaker BE12-615F 50 ml Cosmic Calf Serum (Hyclone SH30087.03), 5 ml of 200 mM/L L-glutamine (Bio Whittalker, BE17-605F), 5 ml penicillin/streptamicin (Bio Whittaker, DE17-603E)) was removed and 800 μl of the preincubated antibody was added to the cells and cells were incubated herewith at 37° C. in an incubator for 15 min, after which 200 μl/well medium or 80 ng/ml rh-HGF was added. (Final concentrations were 10 μg/ml Ab, 2 mg/ml IVIG, 16 ng/ml HGF).

After incubation for another 15 min, the incubation medium was removed and the cells were washed twice with ice cold PBS, and 250 μl RIPA lysis buffer (containing 50 mM Tris, pH 7.5, 0.5% Na deoxycholate and 0.1% Nonidet P40, 150 mM NaCl, 0.1% SDS, 2 mM vanadate and Complete (Protease inhibitor, Roche 1836170) was added, and the plate was gently rotated for 10 min. at 4° C. The lysates were transferred into pre-cooled tubes (Eppendorf) and centrifuged at highest speed for 30 min. at 4° C. DNA was removed and the lysate was flash frozen in N₂ after a fraction was used to measure BCA protein content analysis (Pierce). Lysates were stored at −80° C. until analysis by Western-blot. 10 μg reduced samples were undergoing electrophoresis on 4-20% Tris-HCl-Criterion Precast gel (Biorad 345-0033) and Western blotting on a nitrocellulose membrane (Biorad 162-0114) according standard procedures. The membrane was blocked with blocking solution (containing 5% BSA (Roche, 10735086) in TBST (Tris-HCL 20 mM pH 7.5, NaCl 150 mM, 0.1% Tween 20) for 1.5 hours at room temperature on a roller bank. The membrane was incubated over night at 4° C. with 1:1000 dilution of anti-phospho-Met(pYpYpY 1230 1234 1235)-rabbit IgG, (Abcam, ab5662). After washing 6 times with TBST, the secondary antibodies, goat-anti-rabbit-HRP, Cell Signalling, 7074 (1:2000) in blocking reagent were incubated for 60 min. at room temperature on a roller bank. The membrane was washed 6 times with TBST. Finally the bands were developed with Luminol Echancer stopsolution (Pierce 1856145) and analyzed on a Lumiimager.

cMet-HG Pre-Incubated with IVIG Inhibits the HGF Mediated Phosphorylation of the Receptor.

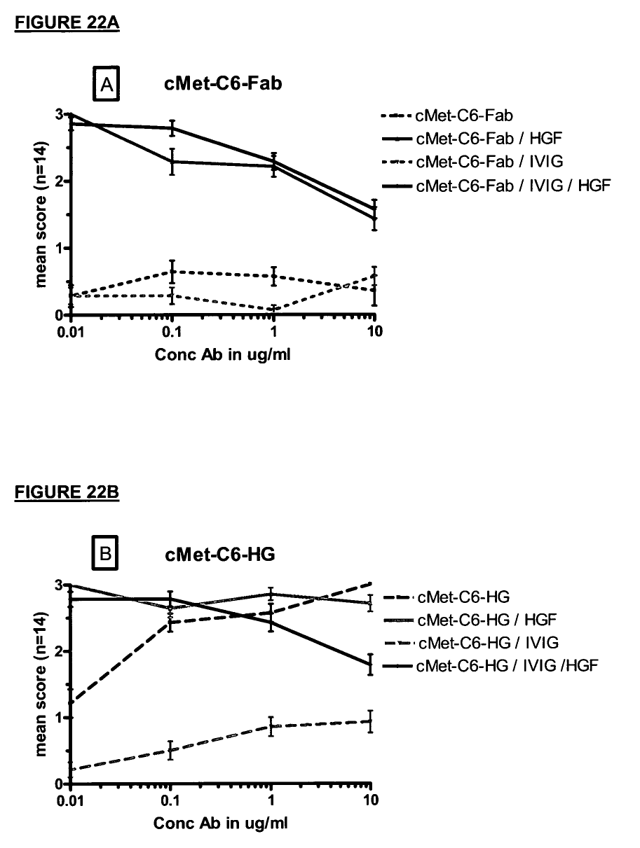

FIG. 22

DU-145 cells were cultured and incubated with a serial dilution of (A) cMet-Fab, cMet-Fab and IVIG, cMet-Fab and HGF, cMet-Fab and IVIG and HGF (B) cMet-HG, cMet-HG and IVIG, cMet-HG and HGF, cMet-HG and IVIG and HGF. Scattering was observed double-blinded (scored by 14 people) by microscope after 48 h and the averaged score±SEM is plotted.

cMet-Fab with or without IVIG (A) and cMet-HG pre-incubated with IVIG (B) significantly blocked the HGF induced scattering dose-dependently.

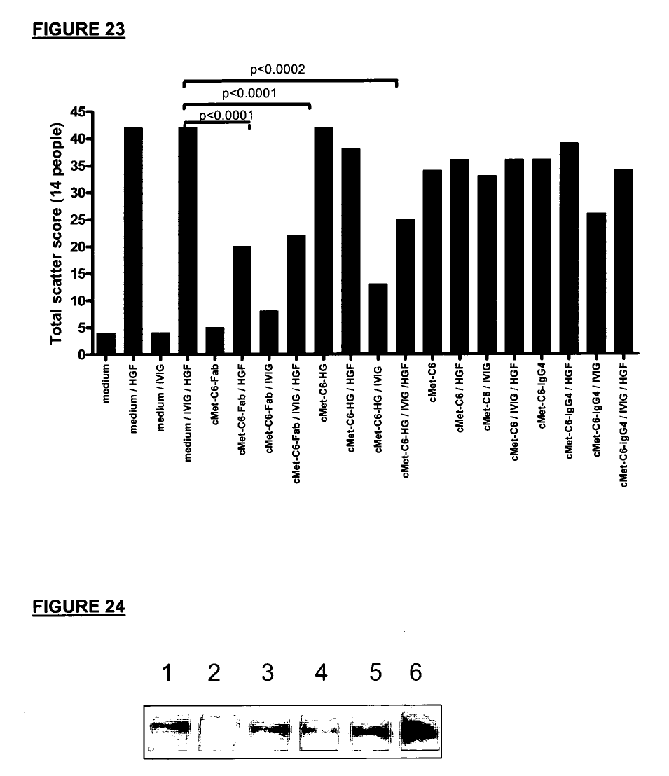

FIG. 23

DU-145 cells were cultured and incubated with 10 μg/ml of (A) cMet-Fab, cMet-Fab and IVIG, cMet-Fab and HGF, cMet-Fab and IVIG and HGF (B) cMet-HG, cMet-HG and IVIG, cMet-HG and HGF, cMet-HG and IVIG and HGF. Scattering was observed double-blinded (scored by 14 people) by microscope after 48 h.

cMet-Fab with or without IVIG and cMet-HG pre-incubated with IVIG significantly inhibited the HGF induced scattering. For statistical analysis a two-tailed Wilcoxon signed ranked test was done with a hypothetical median value of 3 (maximal scattering).

FIG. 24

Extracts prepared from A549 cells incubated with cMet-HG (lane 1), cMet-HG and IVIG (lane 2), cMet-HG and HGF (lane 3), cMet-HG, IVIG and HGF (lane 4), cMet-IgG1 (lane 5), cMet-IgG1 and IVIG (lane 6) were resolved by SDS-PAGE on a 4-20% Tris-HCl-Criterion Precast gel and Western blotting on a nitrocellulose membrane. The membrane was incubated over night at 4° C. with anti-phospho-Met(pYpYpY 1230 1234 1235)-rabbit IgG, (Abcam, ab5662). After washing with TBST, the secondary antibodies, goat-anti-rabbit-HRP, Cell Signalling, 7074 in blocking reagent were incubated for 60 min. at room temperature on a roller bank. The membrane was washed 6 times with TBST. Finally the bands were developed with Luminol Echancer stop solution and analyzed on a Lumiimager. The Western blot shows a 169 Kd band indicating phospho-Met(pYpYpY 1230 1234 1235).

### Example 57

### In Vitro Evaluation of an IgG4 Hingeless Mutant Antibody Targeting the Epidermal Growth Factor Receptor (EGFr): Binding Avidity and Induction of Antibody Dependent Cell-Mediated Cytotoxicity (ADCC)

In this experiment an IgG4 hingeless mutant antibody targeting the Epidermal Growth Factor Receptor (EGFr), mAb 2F8-HG was compared to an IgG4 version, an IgG1 version and Fab fragments, referred to as 2F8-IgG4, 2F8-IgG1 and 2F8-Fab, respectively. The in vitro evaluation comprised the avidity of binding to EGFr in an ELISA and the induction of ADCC.

ELISA. Binding affinities were determined using an ELISA in which purified EGF-R (Sigma, St Louis, Mo.) was coated to 96-well Microlon ELISA plates (Greiner, Germany), 50 ng/well. Plates were blocked with PBS supplemented with 0.05% Tween 20 and 2% chicken serum. Subsequently, samples, serially diluted in a buffer containing 100 μg/ml polyclonal human IgG (Intravenous Immunoglobulin, IVIG, Sanquin Netherlands) were added and incubated for 1 h at room temperature (RT). Plates were subsequently incubated with peroxidase-conjugated rabbit-anti-human kappa light chain (DAKO, Glostrup, Denmark) as detecting antibody and developed with 2,2′-azino-bis(3-ethylbenzthiazoline-6-sulfonic acid) (ABTS; Roche, Mannheim, Germany). Absorbance was measured in a microplate reader (Biotek, Winooski, Vt.) at 405 nm.

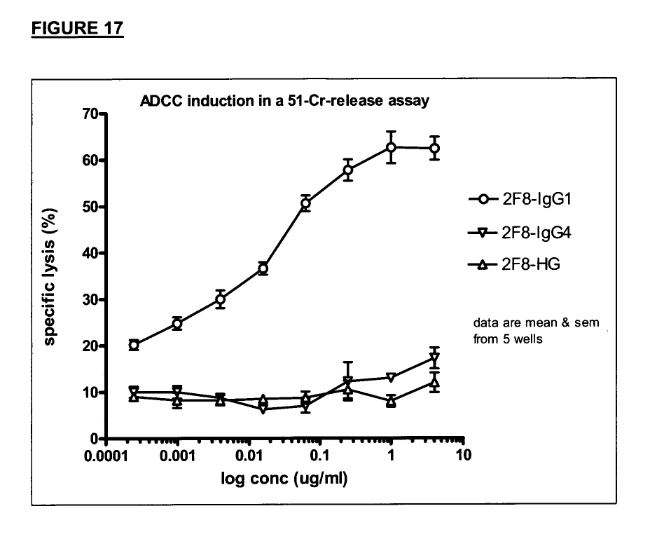

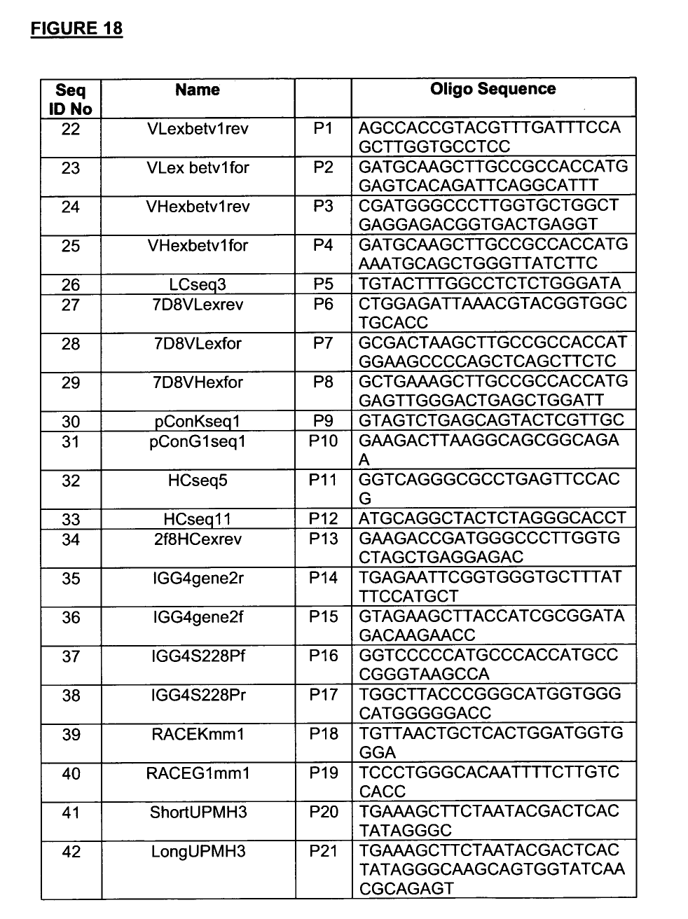

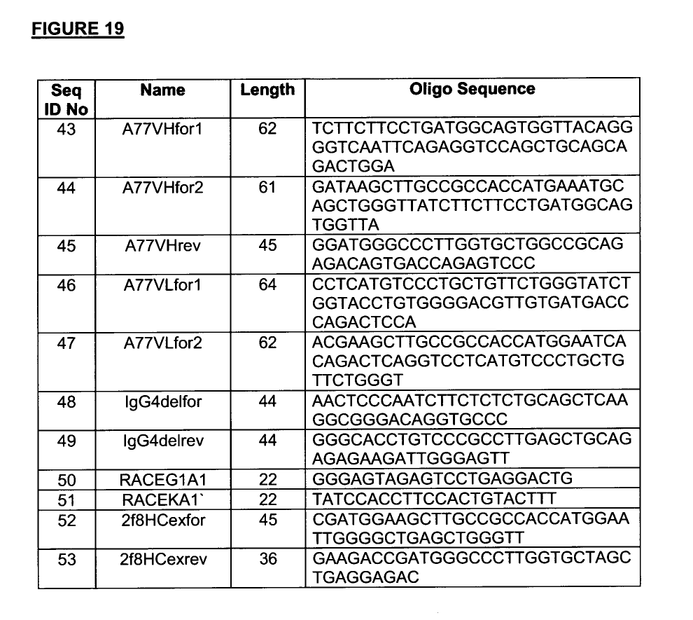

FIG. 16 shows that the binding curves of the 2F8-HG and 2F8-Fab are super-imposable and clearly right-shifted with respect to the binding curves of IgG1 and IgG4. This difference in avidity for the EGFr coat is consistent with the idea that, in the presence of IVIG, 2F8-HG binds monovalently, just like Fab fragments.

Antibody dependent cell-mediated cytotoxicity (ADCC). The capacity to induce effector cell-dependent lysis of tumor cells was evaluated in Chromium-51 (⁵¹Cr) release assay. Target A431 cells (2-5×106 cells) were labeled with 100 μCi Na₂⁵¹CrO₄ (Amersham Biosciences, Uppsala, Sweden) under shaking conditions at 37° C. for 1 h. Cells were washed thrice with PBS and were re-suspended in culture medium 1×10⁵ cells/ml. Labeled cells were dispensed in 96 wells plates (5×10³, in 50 μl/well) and pre-incubated (RT, 30 minutes) with 50 μl of 10-fold serial dilutions of mAb in culture medium, ranging from 20 μg/ml to 0.02 ng/ml (final concentrations). Culture medium was added instead of antibody to determine the spontaneous ⁵¹Cr release, tritonX100 (1% final concentration) was added to determine the maximal ⁵¹Cr release. Thereafter, PBMC were added to the wells (5×10⁵/well) and cells were incubated at 37° C. overnight. The next day, supernatants were collected for measurement of the ⁵¹Cr release by determination of the counts per minute (cpm) in a gamma counter. Percentage of cellular cytotoxicity was calculated using the following formula:

% specific lysis=(experimental release (cpm)−spontaneous release (cpm))/(maximal release (cpm)−spontaneous release (cpm))×100

where maximal ⁵¹Cr release determined by adding triton X-100 to target cells, and spontaneous release was measured in the absence of sensitizing antibodies and effector cells.

FIG. 17 shows that 2F8-HG induces no ADCC, like 2F8-IgG4, whereas 2F8-IgG1 is very potent in this respect.

### Example 58

AlgoNomics' Epibase® platform was applied to IgG4 constant hingeless monovalent antibody. In short, the platform analyzes the HLA binding specificities of all possible 10-mer peptides derived from a target sequence (Desmet et al. 1992, 1997, 2002, 2005). Profiling is done at the allotype level for 20 DRB1, 7 DRB3/4/5, 14 DQ and 7 DP, i.e. 48 HLA class II receptors in total.

Epibase® calculates a quantitative estimate of the free energy of binding □Gbind of a peptide for each of the 48 HLA class II receptors. These data are then further processed as follows: Peptides are classified as strong (S), medium (M), weak and non (N) binders.

No strong and only 1 medium binding epitope was encountered within the constant region of IgG4 hingeless monovalent antibody. This single neo-epitope created a medium DRB1*0407 binder. DRB1*0407 is a minor allotype, present in less than 2% of the Caucasian population. In addition, a single epitope of medium strength is insignificant in the total epitope count of even the least immunogenic antibody.

In conclusion the hingeless monovalent IgG4 antibody is predicted to be very unlikely to be immunogenic.

### Example 59

### Background of Studies and Materials Used in Examples 59 and 60 Presented for Unibody-CD4

In vitro and in vivo experiments were performed to address the ability of a human monoclonal antibody against CD4 (HuMax-CD4) to inhibit HIV-1 infection. The antibody is directed against domain 1 of CD4 and overlaps with the HIV-1 gp120 binding site on CD4. The present example (59) shows that Fab fragments of anti-CD4 antibodies inhibits the infection of CD4-CCR5 cells or CD4-CXCR4 cells by different primary isolates and T-cell line adapted HIV viruses. The IC50 values of inhibition are in the range of the EC50 values of HuMax-CD4 binding to sCD4 and cell bound CD4 (data not shown), implicating inhibition of HIV-1 envelope binding to CD4 as a mechanism of inhibition. In general Fab fragments of HuMax-CD4 inhibit with a 10 times lesser efficiency than the whole antibody which is as expected from the difference in avidity between the Fab and the whole antibody.

Example 60 shows that in mice treated with HuMax-CD4 a lesser decline in CD4/CD8 ratio compared is observed than in IgG control treatment groups, indicating that HuMax-CD4 protects against depletion of CD4 positive cells by HIV-1. Furthermore, HuMax-CD4 treatment leads to a decrease in the amount of HIV-1 RNA copies in the blood in time, whereas the IgG control treatment does not induce this decrease. The in vitro data indicate that anti-CD4 antibodies can protect against HIV-1-induced CD4 depletion, and decrease the magnitude of HIV infection and viral load.

Norris et al have published on the treatment of HIV-1 infected individuals with a whole anti-CD4 (domain 2) antibody of the IgG4 subclass.

Efficacy results demonstrated significant antiviral activity at primary endpoint (Week 24).

Durable response suggested by Week-48 results in patients receiving TNX-355.

TNX-355 10 mg/kg+OBR demonstrated a 0.96 log 10 reduction in HIV-RNA from baseline at Week 48 versus 0.14 log 10 decrease for placebo+OBR (p<0.001).

TNX-355 15 mg/kg+OBR demonstrated a 0.71 log 10 reduction in HIV-RNA from baseline at Week 48 versus 0.14 log 10 for placebo+OBR (p=0.009).

Treatment with TNX-355+OBR was associated with statistically significant and clinically-meaningful increases in CD4+ cells at Week 48 in both the 10 mg/kg arm (+48 cells, p=0.031) and the 15 mg/kg (+51 cells, p=0.016) arms versus the placebo increase (+1 cell).

### LITERATURE

Zwick M. B., Wang M., Poignard P., Stiegler G., Katinger H., Burton D. R., and Parren P. W. H. I. 2001. Neutralization synergy of human immunodeficiency virus type 1 primary isolates by cocktails of broadly neutralizing antibodies. J Vir 75:12198.
Poignard P., Sabbe R., Picchio G. R., Wang M., Gulizia R. J., Katinger H., Parren P. W. H. I., Mosier D. E., and Burton D. R. 1999. Neutralizing antibodies have limited effects on the control of established HIV-1 infection in vivo. Immunity 10:431.
Norris D., Moralis J., Gathe J., Godafsky E., Garcias F., Hardwick R., and Lewis S. 2006. Phase 2 efficacy and safety of the novel viral-entry inhibitor, TNX-355, in combination with optimized background regimen (OBR). XVI International AIDS Conference, Toronto, Canada.

### In Vitro HIV-1 Neutralization by Humax-CD4 Whole Antibody and Fab Fragments of the Humax-CD4 Antibody

The method is described in detail in Zwick et al 2001. In summary, the degree of virus neutralization by antibody was measured by luciferase activity. Viruses competent for a single round of replication were produced by cotransfections of the appropriate virus constructs in a modified pSVIIIenv vector (for instance primary isolates: JR-CSF, JR-FL, SF162, ADA, YU2, 89.6, US143 and T cell line adapted virus: IIIB) and pNL4-3.lec.R-E-. Viruses were pre-incubated with various amounts of antibody (before addition determined to yield about 100,000 counts) to U87.CD4.CCR5 cells (primary isolates) or CD4-CXCR4 cells (for IIIB), and culturing for 3 days. The wells were washed, incubated with luciferase cell culture lysis reagent, and lysates were transferred to opaque assay plate to measure luciferase activity on a luminometer using luciferase assay reagent. For neutralization HuMax-CD4 and Fab fragments of HuMax-CD4 were tested.

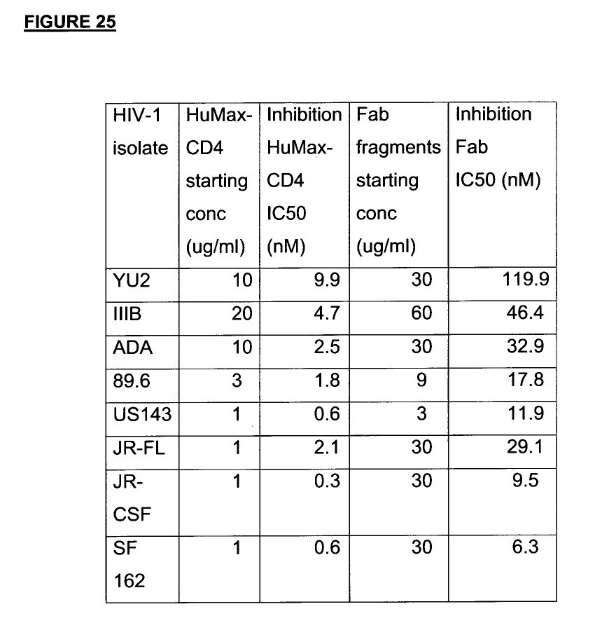

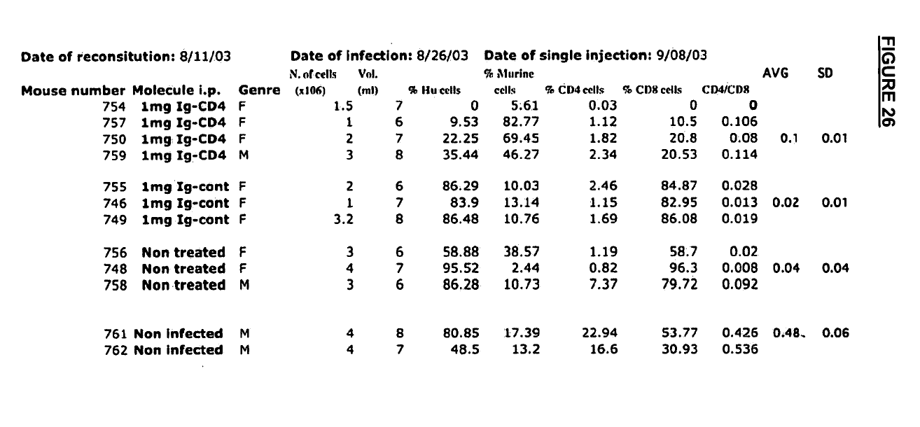

According to the method described, the virus constructs YU2, IIIB, ADA, 89.6, US143, JR-FL, JR-CSF, and SF 162 were used in the in vitro neutralization assay using the luciferase assay expression system. HIV-1 IIIB is a T-cell line adapted virus, all the other viruses are primary isolates of HIV-1. The HuMax-CD4 antibody and Fab fragments of HuMax-CD4 were added in a 1:2 dilution response starting at the concentrations indicated in FIG. 25. In FIG. 27, the curves fitted by a 4 parameter logistic analysis are given for the HuMax-CD4 and the Fab fragments of HuMax-CD4 and in FIG. 25 the IC50 calculated from these fits are indicated. The data show that the HuMax-CD4 antibody inhibited the infection of all the viruses tested, and in general did this with a 10 times better efficiency than the Fab fragments (exceptions are YU2 and JR-CSF). The EC50 for binding of HuMax-CD4 to sCD4 has been determined to be about 0.3-1 nM. The IC50 values of inhibition are in the range of these EC50 values, indicating that receptor occupation by HuMax-CD4 relates to degree of infection inhibition.

Our experiments provide proof-of-principle for an effective inhibition of HIV-1 infection of both CXCR4 and CCR5HIV-1 co-receptor expressing cells by monovalent binding of an anti-CD4 antibody (i.e. Fab fragment). This provides evidence that a similar inhibition could be accomplished by a HG anti-CD4 antibody.

### Example 60

### Protection of CD4+ T Cell Depletion in In Vivo hu-PBMC-SCID Mouse Model of HIV Infection

The experimental procedure is described in detail in Poignard et al 1999. In summary, CB-17 SCID mice were reconstituted with about 25×10⁶ normal human PBMC (peripheral blood mononuclear cells). About two weeks later the animals were infected with HIV-1 (HIV-1_{JR-CSF}). Three days later the animals are treated with 1 mg/ml HuMax-CD4, or a human IgG isotype control antibody, or no treatment delivered intraperitoneally. Blood samples were taken at 1 hr, 6 hrs, day 1, 2, 3, 6, 9, 13, and 15 after injection, and two weeks later the animals were euthanized and FACS analysis performed to determined the % of human cells (using H2 Kd-PE and human CD3-APC) and the CD4/CD8 ratio (using CD4-PE and CD8-APC double staining). Furthermore, plasma viral load was measured by measuring HIV-1 RNA levels by the quantitative Roche RT PCR assay. In addition, with a direct sCD4 binding ELISA (coat of sCD4 on the plate, and detection by anti-Fc polyclonal antibody) the concentrations of HuMax-CD4 in plasma were determined.

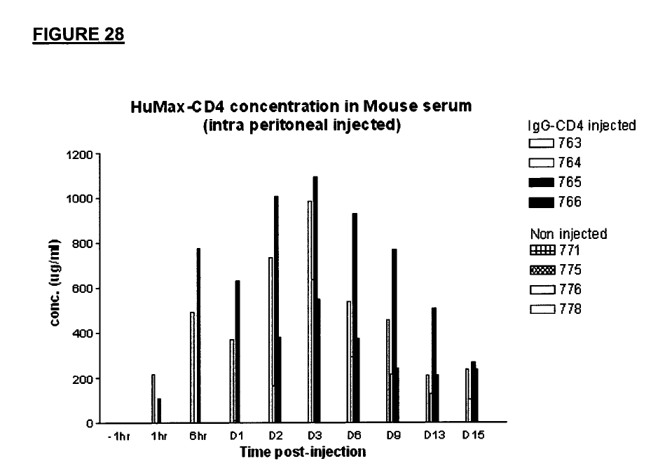

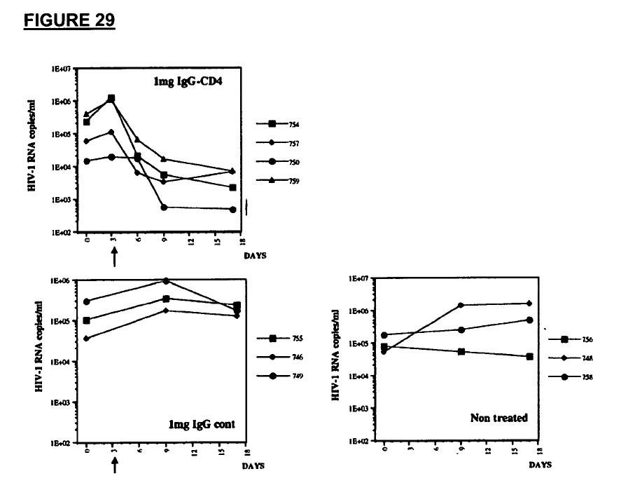

In FIG. 28 the plasma levels of the animals are given. It is concluded that HuMax-CD4 injection leads to high HuMax-CD4 plasma concentrations that were still above 100 μg/ml at day 15. The non treated mice gave no measurable values above background.

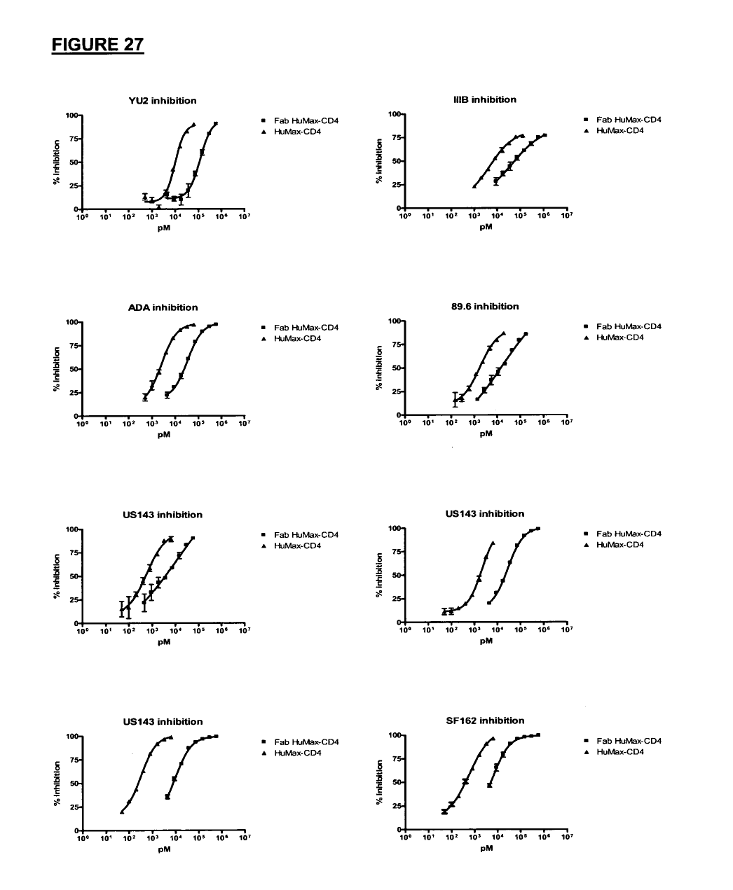

In FIG. 26 the cell numbers harvested from the mice at the end of the experiment are given. The data indicate that HIV-1 infection led to an extensive decrease in CD4 positive T cells as indicated by the drop in CD4/CD8 ratio. This shows that CD4 positive T cells are rapidly depleted from the blood by HIV-1 in contrast to the constant levels in non-infected mice. The mice treated ip with HuMax-CD4 had a much smaller decline in CD4/CD8 ratio, which shows that HuMax-CD4 provides protection of against depletion of CD4 positive cells by HIV-1. In FIG. 29 the HIV-1 RNA copies per ml blood are given in time, and these data indicate that the HuMax-CD4 treatment led to a decrease in the amount of HIV-1 RNA copies in the blood in time, whereas the isotype control antibody did not lead to a decrease.

Our experiment provides proof of principle for the protection against CD4 cell depletion in HIV-1 infection in vivo. The protection against depletion is observed even though the whole anti-CD4 antibody has CD4 depleting properties it self. This indicates that stronger protection against HIV-1-induced T cell depletion can be obtained by treatment with a monovalent non-depleting anti-CD4 antibody such as an anti-CD4 HG antibody. Proof of principle for HIV-1 neutralization by anti-CD4 HG and protection against CD4 depletion can be obtained in a similar experimental set-up. This provides evidence that HuMax-CD4 HG showing a long in vivo half life, could inhibit HIV-1 infection and HIV-1 viral load and protect from depletion of CD4 positive cells.

### Example 61

### Using a Monovalent Antibody or a Fragment Thereof as Fusion Partner for Elongation of Half-Life

Apoptosis has been suggested as one of the major mechanisms of CD4+ T-cell depletion during the course of HIV-1 infection. Interleukin-7 (IL-7), a non-redundant cytokine that plays essential roles in T-cell homeostasis, has been shown to have a anti-apoptotic effect ex vivo on both CD4+ and CD8+ T-cells derived from HIV-1 infected patients (Vassena, L, et al; PNAS 2007).

Proof of principle in vivo of a UniBody used as a fusion partner increasing the half-live of a protein is obtained in the following experiment. The coding region of IL-7 is amplified from a plasmid containing this region using specific primers and introducing suitable restriction sites. To make a construct for expression of a IL-7-UniBody fusion protein, the IL-7 coding region is digested with the suitable restriction enzymes and cloned into the pTomG47D8HG (Example 33), replacing the VH and CH1 domain of 7D8 using standard cloning techniques (Sambrook J. and Russel, D. V. Molecular Cloning: A Laboratory Manual, 3^{rd} Ed., Cold Spring Harbor, 2000). The fusion protein is expressed and purified as described previously (Examples 12 and 40, respectively).

The experimental procedure is described in detail in Poignard et al 1999. In summary, CB-17 SCID mice are reconstituted with about 25×10⁶ normal human PBMC (peripheral blood mononuclear cells). About two weeks later the animals are infected with HIV-1 (HIV-1_{JR-CSF}). Three days later the animals are treated with 1 mg IL-7-UniBody fusion protein, recombinant IL-7 (equimolar amount) or no treatment delivered intraperitoneally.

Blood samples are taken at 1 hr, 6 hrs, day 1, 2, 3, 6, 9, 13, and 15 after injection, and two weeks later the animals are euthanized and FACS analysis is performed to determine the % of human cells (using H2 Kd-PE and human CD3-APC) and the CD4/CD8 ratio (using CD4-PE and CD8-APC double staining). Additionally, apoptosis markers (AnnexinV-FITC and TO-PRO-3 staining) are determined. Furthermore, plasma viral load is measured at each time-point by measuring HIV-1 RNA levels by the quantitative Roche RT PCR assay in blood samples. In addition, with a capture ELISA, the concentrations of IL-7 and IL-7-UniBody fusion protein is measured at each time-point in blood samples.

### Summary of the Results

The data presented in the examples shows that expression of a hingeless IgG4 antibody by destroying the splice donor site of the hinge exon results in hingeless IgG4 half-molecules (one heavy and one light chain combined). The presence of IgG4 hingeless half-molecules is confirmed by SDS-PAGE under non-reducing conditions, mass spectrometry, size exclusion chromatography and radio immuno assay the absence of cross-linking abilities. The hingeless antibodies retain the same antigen binding specificity as natural format IgG1 and IgG4 antibody molecules. This is shown for two hingeless antibodies with different specificity, 7D8-HG (specific for the B-cell antigen CD20) and Betv1-HG (specific for the Birch pollen antigen Bet v 1). C1q binding of 7D8-HG is absent and only minor complement-dependent cellular toxicity (ADCC) is observed (comparable to the natural format 7D8-IgG4 antibody). Monovalency of the hingeless half-molecule is shown in the crosslinking experiment using Betv1-HG. Whereas both IgG1 and IgG₄ show crosslinking of Sepharose bound Bet v 1 to radiolabelled Bet v 1, the hingeless molecule Betv1-HG is unable to crosslink.

Half-life of 7D8-HG is evaluated in vivo in a mouse pharmacokinetic (PK) experiment and compared with 7D8-IgG4. Although 7D8-HG has a 2 to 3 times faster clearance than normal IgG4 in this model, the 6 day half-life is counted favorable to the half-life of less than one day reported for IgG F(ab′)2 fragments. We conclude that the favorable PK-profile will make IgG4-hingeless antibodies valuable for therapeutic applications when a non-crosslinking, monovalent and non-complement-activating antibody is needed.

The use of monovalent immunoglobulins of the invention as a fusion partner to prolong the in vivo half life of other molecules has been described.

### Example 62

### Constructions and Biochemical Analysis of CH3 Variants of 2F8-HG

To prevent dimerization irrespective of the presence of irrelevant antibodies, additional mutations were introduced into the CH3 region. To make the constructs for the expression of the CH3 mutants, the mutations were introduced into pTomG42F8HG using site-directed mutagenesis. The constructs were expressed transiently.

In order to investigate whether CH3 variant HG molecules exist as monomers or dimers, a mass spectrometry method was employed as described above.

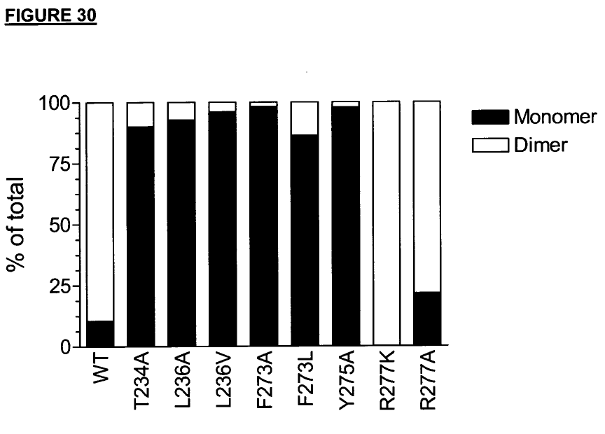

FIG. 30 shows a summary of the monomer/dimer ratios obtained for each HG mutant using non-covalent nano-electrospray mass spectrometry. CH3 mutants showed a substantial increase in monomer/dimer ratio compared to 2F8-HG (WT). The percentage molecules present as monomers increased from 15% in 2F8-HG (WT) to >80% in most CH3 mutants, except for mutation R277A. HG mutation R277K, which introduces an IgG1 sequence into the IgG4 backbone, was used as negative control. As expected, this mutant behaved as dimer.

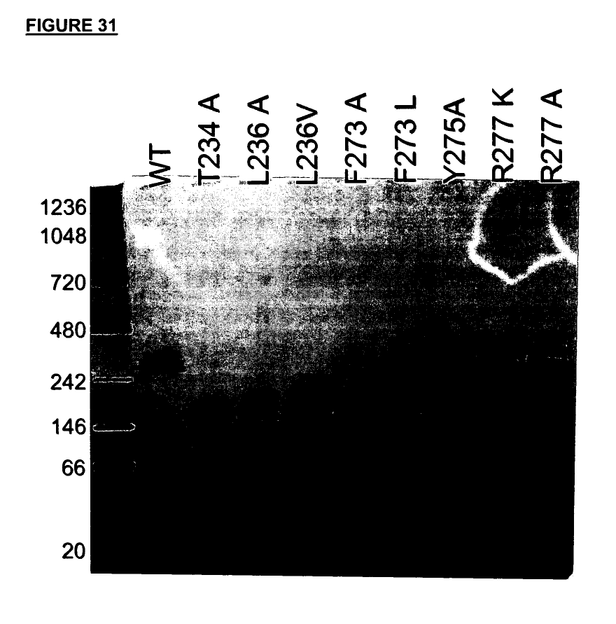

The monomer or dimer configuration of CH3 mutants was verified using NativePAGE™ Novex® Bis-Tris gel electrophoresis (Invitrogen, Carlsbad, Calif.) according to the instructions of the manufacturer as shown in FIG. 31. This native gel electrophoresis technique uses Coomassie G-250 as a charge-shift molecule instead of SDS and is able to maintain native protein conformation and protein complex quaternary structures (Schagger H and von Jagow G 1991 Blue native gel electrophoresis for isolation of membrane complexes in enzymatically active form. Anal. Biochem. 199:223-244).

Under these experimental conditions, 2F8-HG (WT) and R277K and R277A showed a protein band corresponding to the size of a full tetrameric (two heavy and two light chains) molecule. The CH3 mutants T234A, L236A, L236V, F273A, F273L, and Y275A were shown to be half molecules (only one heavy and one light chain).

### Example 63

### Functional Analysis of CH3 Mutants of 2F8-HG

Binding of 2F8-HG (WT) and variants was determined in the absence and presence of 200 μg/ml polyclonal human IgG (Intravenous Immunoglobulin, IVIG, Sanquin Netherlands) (as described in Example 57).

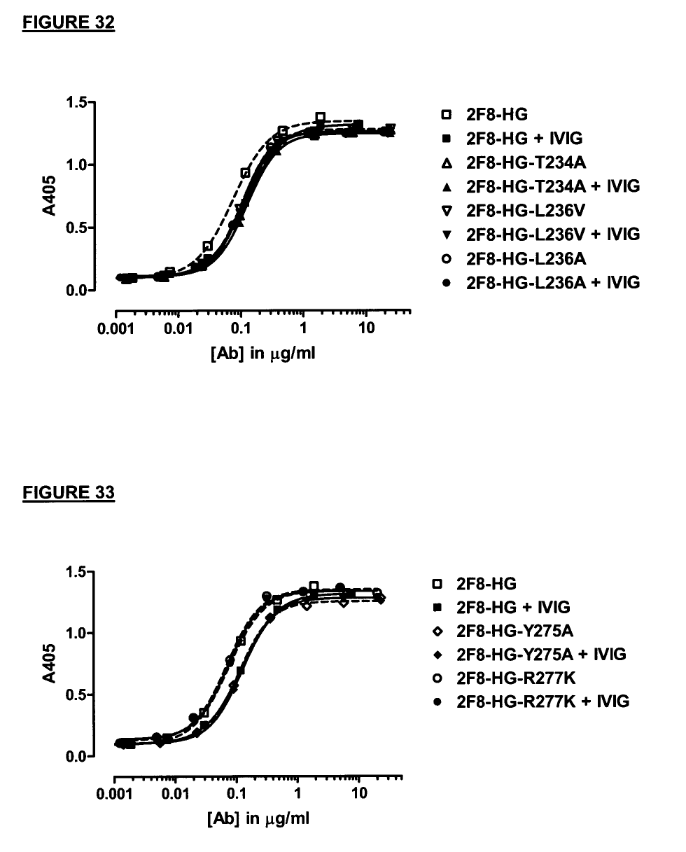

FIGS. 32 and 33 show that the binding curve of 2F8-HG in the presence of IVIG clearly right-shifts with respect to the binding curve of 2F8-HG without IVIG. This difference in avidity for the EGFr coat is consistent with the idea that, in the presence of IVIG, 2F8-HG binds monovalently (see Example 57). The binding curves of several of the tested mutations, 2F8-HG-T234A, 2F8-HG-L236V, 2F8-HG-L236A and 2F8-HG-Y275A, become insensitive to the addition of IVIG and were super-imposable on the monovalent binding curve of 2F8-HG in the presence of IVIG. These differences in avidity for the EGFr coat are consistent with the idea that the 2F8-HG-T234A, 2F8-HG-L236V, 2F8-HG-L236A and 2F8-HG-Y275A mutations prevent dimerization of the HG molecules.

### Example 64

### Functional Analysis of CH3 Mutants of 2F8-HG

CH₃ mutants of 2F8-HG were shown to bind EGFr with lower apparent affinities than 2F8-HG in a binding ELISA coated with EGFr protein (see above). The potency of 2F8-HG CH3 mutants to inhibit ligand-induced EGFr phosphorylation in cells in vitro was compared to that of 2F8-HG (WT) and 2F8-Fab fragments in the Phosphorylation Inhibition Assay (PIA) as described in example 54.

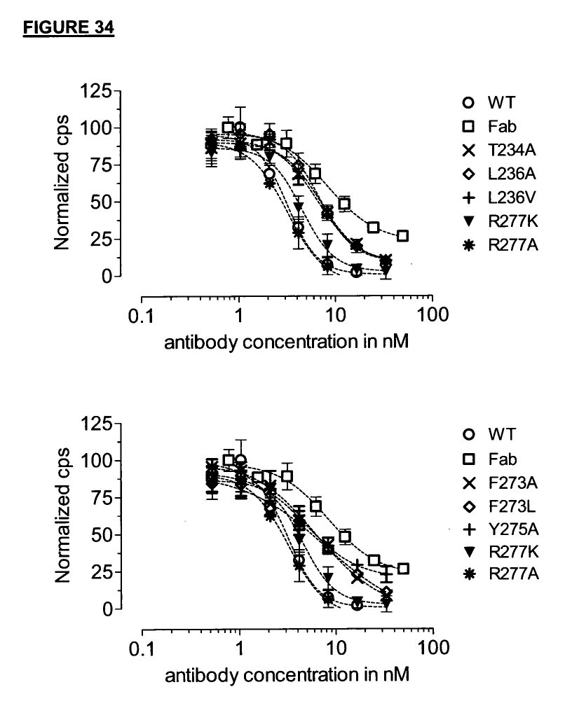

CH3 HG mutants were less potent to inhibit EGFr phosphorylation than 2F8-HG (WT) and the control mutants R277K and R277A, in line with the increase in monomer/dimer ratio of these mutants (FIG. 34).

### Example 65

### Concentration Dependent Configuration of CH3 Mutants of HG

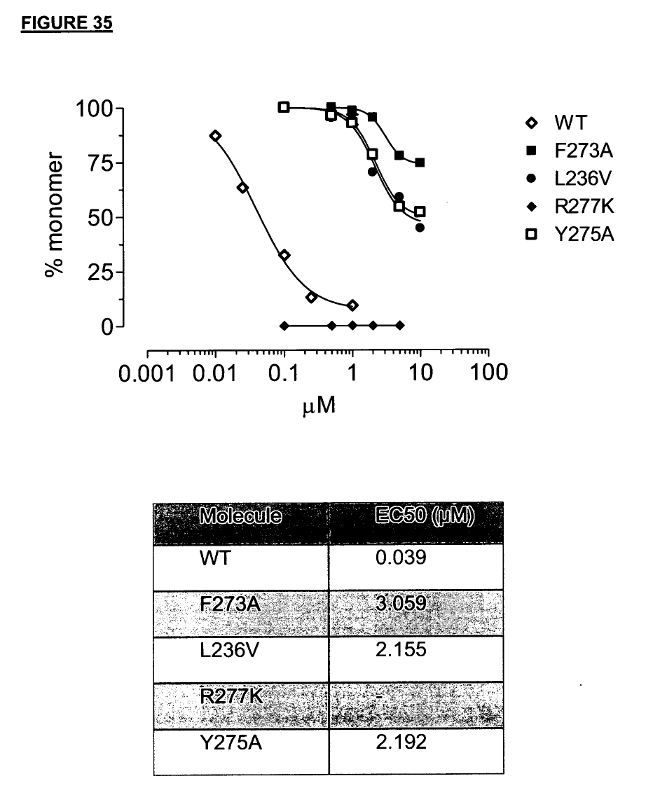

The monomer/dimer configuration of CH3 mutants F273A, L236V, and Y275A was further investigated at different concentrations, ranging from 0.01-10 μM using non-covalent nano-electrospray mass spectrometry as described above. The monomer/dimer configuration of these CH3 mutants was compared to the configuration of 2F8-HG (WT) and R277K. The percentage molecules present as monomers at each concentration were plotted and EC50 values were calculated for each mutant (FIG. 35).

All HG mutants were 100% monomeric at low concentrations (except for R277K which behaved as dimer). With increased concentration of HG mutants, a decrease in monomericity was observed. However, the figure shows that the CH3 mutants exhibited such decrease in monomericity at much higher concentration than 2F8-HG (WT). Hence, the CH3 mutants contained a higher percentage of monomer molecules at higher molar concentrations.

1. A fusion protein comprising at least a first molecule and a monovalent immunoglobulin or a fragment of a monovalent immunoglobulin, wherein i) said first molecule and said monovalent immunoglobulin or fragment thereof are fused or linked either by peptide bonds or by other types of covalent bonding, ii) the monovalent immunoglobulin or the fragment thereof comprises at least the CH2 and CH3 regions of the CH, iii) the monovalent immunoglobulin or fragment thereof, as required by the Ig subtype, has been modified such that the CH3 region or other regions do not comprise any amino acid residues which are capable of participating in the formation of disulphide bonds or covalent or stable non-covalent inter-heavy chain bonds with other peptides comprising an identical amino acid sequence of the CH region of the immunoglobulin in the presence of polyclonal human Ig; iv) if a CL region or a fragment of a CL region is not present, said monovalent immunoglobulin or fragment thereof does not comprise the CH1 region or has a CH1 region wherein one or more amino acids in the hydrophobic patch have been changed to amino acids that are less hydrophobic, unless the CH1 region is of the camelidae type, and v) the hinge region of the CH region is either deleted or mutated to remove any cysteine residues.

2. A fusion protein according to claim 1, wherein the first molecule is one of the following: a polypeptide such as a cytokine, a peptide mimetic, a small organic molecule.

3. A fusion protein according to claim 1, wherein the monovalent immunoglobulin or fragment thereof does not comprise a VH or VL region or a fragment thereof.

4. A fusion protein according to claim 1, wherein the hinge region of the monovalent immunoglobulin or fragment thereof has been deleted.

5. A fusion protein according to claim 1, wherein the first molecule is a polypeptide which is fused to the N-terminus of the monovalent immunoglobulin or fragment thereof.

6. A fusion protein according to claim 1, wherein a CL region or a fragment of a CL region is present.

7. (canceled)

8. A fusion protein according to claim 1, wherein the first molecule comprises two polypeptides fused to the monovalent immunoglobulin or fragment thereof, wherein the one polypeptide is fused or linked to the heavy chain of the monovalent immunoglobulin and the other polypeptide is fused or linked to the light chain.

9. A fusion protein according to claim 8, wherein the two polypeptides fused to the monovalent immunoglobulin are different.

10. A fusion protein according to claim 1, wherein i) the monovalent immunoglobulin or fragment thereof comprises an variable region, and wherein the fusion protein comprises ii) a linker molecule comprising one part that is capable of being bound by said variable region, and a second part that is capable of binding to the first molecule, and iii) a first molecule which is capable of being bound by the linker of ii).

11. A fusion protein according to claim 1, wherein the first molecule is fused to the monovalent immunoglobulin or fragment thereof by peptide bonding, and wherein one or more amino acids have been inserted as spacers between the polypeptide and the monovalent immunoglobulin or fragment thereof.

12. A fusion protein according to claim 1, wherein the first molecule is covalently linked to the monovalent immunoglobulin or fragment thereof by other covalent bonds than peptide bonding, and wherein a linker molecule is inserted between the two molecules.

13. A fusion protein according to claim 1, wherein the Ig is IgG1, IgG2, IgG3, IgG4, IgA1, IgA2 or IgD.

14. A fusion protein according to claim 1, wherein the Ig is human.

15. A fusion protein according to claim 14, wherein the human Ig comprises the amino acid CH 3 region as set forth in SEQ ID NO: 19, wherein the CH 3 region has been modified so that one or more of the following amino acid substitutions have been made: Arg (R) in position 238 has been replaced by Gln (Q); Asp (D) in position 239 has been replaced by Glu (E); Thr (T) in position 249 has been replaced by Ala (A); Leu (L) in position 251 has been replaced by Ala (A); Leu (L) in position 251 has been replaced by Val (V); Phe (F) in position 288 has been replaced by Ala (A); Phe (F) in position 288 has been replaced by Leu (L); Tyr (Y) in position 290 has been replaced by Ala (A); Lys (K) in position 292 has been replaced by Arg (R); Lys (K) in position 292 has been replaced by Ala (A); Gln (Q) in position 302 has been replaced by Glu (E); and Pro (P) in position 328 has been replaced by Leu (L).

16. A fusion protein according to claim 15, wherein Lys (K) in position 292 has been replaced by Arg (R).

17. A fusion protein according to claim 14, wherein the human Ig comprises the amino acid CH3 region as set forth in SEQ ID NO: 20, wherein the CH 3 region has been modified so that one or more of the of the following amino acid substitutions have been made: Arg (R) in position 234 has been replaced by Gln (Q); Thr (T) in position 245 has been replaced by Ala (A); Leu (L) in position 247 has been replaced by Ala (A); Leu (L) in position 247 has been replaced by Val (V); Met (M) in position 276 has been replaced by Val (V); Phe (F) in position 284 has been replaced by Ala (A); Phe (F) in position 284 has been replaced by Leu (L); Tyr (Y) in position 286 has been replaced by Ala (A); Lys (K) in position 288 has been replaced by Arg (R); Lys (K) in position 288 has been replaced by Ala (A); Gln (Q) in position 298 has been replaced by Glu (E); and Pro (P) in position 324 has been replaced by Leu (L).

18. A fusion protein according to claim 17, wherein Lys (K) in position 288 has been replaced by Arg (R).

19. A fusion protein according to claim 14, wherein the human Ig comprises the amino acid CH region as set forth in SEQ ID NO: 21, wherein the CH 3 region has been modified so that one or more of the following amino acid substitutions have been made: Arg (R) in position 285 has been replaced by Gln (Q); Thr (T) in position 296 has been replaced by Ala (A); Leu (L) in position 298 has been replaced by Ala (A); Leu (L) in position 298 has been replaced by Val (V); Ser (S) in position 314 has been replaced by Asn (N); Asn (N) in position 322 has been replaced by Lys (K); Met (M) in position 327 has been replaced by Val (V); Phe (F) in position 335 has been replaced by Ala (A); Phe (F) in position 335 has been replaced by Leu (L); Tyr (Y) in position 337 has been replaced by Ala (A); Lys (K) in position 339 has been replaced by Arg (R); Lys (K) in position 339 has been replaced by Ala (A); Gln (Q) in position 349 has been replaced by Glu (E); Ile (I) in position 352 has been replaced by Val (V); Arg (R) in position 365 has been replaced by His (H); Phe (F) in position 366 has been replaced by Tyr (Y); and Pro (P) in position 375 has been replaced by Leu (L).

20. A fusion protein according to claim 19, wherein Lys (K) in position 339 has been replaced by Arg (R)

21. A fusion protein according to claim 1, wherein the monovalent immunoglobulin or fragment thereof comprises the human IgG4 CH2 and CH3 sequences as set forth in SEQ ID NO: 16.

22. A fusion protein according to claim 1, wherein the monovalent immunoglobulin comprises the CH2 and CH3 sequence as set forth in SEQ ID NO: 16. but wherein the CH3 region has been modified so that one or more of the following amino acid substitutions have been made: Thr (T) in position 234 has been replaced by Ala (A); Leu (L) in position 236 has been replaced by Ala (A); Leu (L) in position 236 has been replaced by Val (V); Phe (F) in position 273 has been replaced by Ala (A); Phe (F) in position 273 has been replaced by Leu (L); Tyr (Y) in position 275 has been replaced by Ala (A).

23. The monovalent antibody according to claim 1, wherein the monovalent antibody comprises the CH3 region as set forth in SEQ ID NO: 16.

24. The monovalent antibody according to claim 23, but wherein Glu (E) in position 225 has been replaced by Ala (A).

25. The monovalent antibody according to claim 23, but wherein Thr (T) in position 234 has been replaced by Ala (A).

26. The monovalent antibody according to claim 23, but wherein Leu (L) in position 236 has been replaced by Ala (A).

27. The monovalent antibody according to claim 23.

28. The monovalent antibody according to claim 23, but wherein Leu (L) in position 236 has been replaced by Glu (E).

29. The monovalent antibody according to claim 23, but wherein Leu (L) in position 236 has been replaced by Gly (G).

30. The monovalent antibody according to claim 23, but wherein Lys (K) in position 238 has been replaced by Ala (A).

31. The monovalent antibody according to claim 23, but wherein Asp (D) in position 267 has been replaced by Ala (A).

32. The monovalent antibody according to claim 23, but wherein Phe (F) in position 273 has been replaced by Ala (A).

33. The monovalent antibody according to claim 23, but wherein Phe (F) in position 273 has been replaced by Leu (L).

34. The monovalent antibody according to claim 23, but wherein Phe (F) in position 273 has been replaced by Asp (D) and/or Tyr (Y) in position 275 has been replaced by Glu (E).

35. The monovalent antibody according to claim 23, but wherein Phe (F) in position 273 has been replaced by Thr (T) and/or Tyr (Y) in position 275 has been replaced by Glu (E).

36. The monovalent antibody according to claim 23, but wherein Tyr (Y) in position 275 has been replaced by Ala (A).

37. The monovalent antibody according to claim 23, wherein the monovalent antibody further comprises the CH2 region as set forth in SEQ ID NO: 16, but wherein Thr (T) in position 118 has been replaced by Gln (Q) and/or Met (M) in position 296 has been replaced by Leu (L).

38. The monovalent antibody according to claim 23, wherein the monovalent antibody further comprises the CH2 region as set forth in SEQ ID NO: 16, but wherein one, two or all three of the following substitutions have been made: Met (M) in position 120 has been replaced by Tyr (Y); Ser (S) in position 122 has been replaced by Thr (T); and Thr (T) in position 124 has been replaced by Glu (E).

39. The monovalent antibody according to claim 23, wherein the monovalent antibody further comprises the CH2 region as set forth in SEQ ID NO: 16, but wherein Asn (N) in position 302 has been replaced by Ala (A).

40. The monovalent antibody according to claim 23, wherein the monovalent antibody further comprises the CH2 region as set forth in SEQ ID NO: 16, but wherein Asn (N) in position 302 has been replaced by Ala (A) and Thr (T) in position 175 has been replaced by Ala (A) and Glu (E) in position 248 has been replaced by Ala (A).

41. A fusion protein according to claim 1, wherein the amino acid sequence of a heavy chain of a human IgG4 has been modified such that said heavy chain comprises a CH region, wherein the amino acid residues corresponding to amino acid residues 106 and 109 of the sequence of SEQ ID No: 14 have been deleted or substituted with amino acid residues different from cysteine.

42. A fusion protein according to claim 41, wherein the C H region has been modified such that the cysteine residues of the hinge region have been substituted with amino acid residues that have an uncharged polar side chain, or a nonpolar side chain.

43. A fusion protein according to claim 1, wherein the monovalent immunoglobulin or fragment thereof is a human IgG4, wherein one of the amino acid residues corresponding to amino acid residues 106 and 109 of the sequence of SEQ ID No: 14 has been substituted with an amino acid residue different from cysteine, and the other of the amino acid residues corresponding to amino acid residues 106 and 109 of the sequence of SEQ ID No: 14 has been deleted.

44. A fusion protein according to claim 1, wherein the monovalent immunoglobulin or fragment thereof is a human IgG4, wherein at least the amino acid residues corresponding to amino acid residues 106 to 109 of the C H sequence of SEQ ID No: 14 have been deleted.

45. A fusion protein according to claim 1, wherein the monovalent immunoglobulin or fragment thereof is a human IgG4, wherein at least the amino acid residues corresponding to amino acid residues 99 to 110 of the sequence of SEQ ID No: 14 have been deleted.

46. A fusion protein according to claim 1, wherein the monovalent immunoglobulin or fragment thereof is a human IgG4, wherein the CH region comprises the amino acid sequence of SEQ ID No: 16.

47. A fusion protein according to claim 1, wherein the monovalent immunoglobulin or fragment thereof is a human IgG4, wherein the CH region has been modified such that the entire hinge region has been deleted.

48. A fusion protein according to claim 1, wherein said fusion protein has a plasma concentration above 10 mg/ml for more than 7 days when administered in vivo to a human being or to a SCID mouse at a dose of 4 mg/kg.

49. A fusion protein according to claim 1, wherein said fusion protein has a half-life of at least 5 days when administered in vivo.

50. A fusion protein according to claim 1, wherein said fusion protein has a half-life of at least 5 and up to 21 days when administered in vivo to a human being or a SCID mouse.

51. A fusion protein according to claim 1, wherein said fusion protein is capable of binding to FcRn.

52. A fusion protein according to claim 1, wherein the first molecule linked to the monovalent immunoglobulin or fragment thereof is a small organic molecule.

53. A fusion protein according to claim 1, wherein the first molecule is selected from the list of erythropoietin, thrombopoietin, interferon-alpha (2a and 2b), -beta (1b), -gamma, TNFR I (CD120a), TNFR II (CD120b), IL-I R type 1 (CD121a), IL-1 R type 2 (CD121b), IL-2, IL2R (CD25), IL-2R-beta (CD123), IL-3, IL-4, IL-3R (CD123), IL-4R (CD124), IL-5R (CD125), IL-6R-alpha (CD126), -beta (CD130), IL-10, IL-11, IL-15BP, IL-15R, IL-20, IL-21, TCR variable chain, RANK, RANK-L, CTLA4, CXCR4R, CCR5R, TGF-beta1, -beta2, -beta3, G-CSF, GM-CSF, MIF-R (CD74), M-CSF-R (CD1 15), GM-CSFR (CD1 16), soluble FcgammaRI, sFcgammaRIIa, sFcgammaRIIb, sFcgammaRIIIa, sFcgammaRIIIb, sFcRn, sFcepsilonRI, sFcepsilonRIIa, sFcepsilonRIIb, sFcalphaI, Factor VII, Factor VIII, Factor IX, VEGF, VEGFxxxb, soluble Siglec-1, sSiglec-2, sSiglec-3, sSiglec-4, sSiglec-5, sSiglec-6, sSiglec-7, sSiglec-8, sSiglec-9, sSiglec-10, sSiglec-1 1, sSiglec-12. sSiglec-14 and sSiglec-15.

54. A fusion protein according to claim 1, wherein the first molecule is selected from the list of anti-psychotic drugs, anti-depressant drugs, anti-Parkinson drugs, anti-seizure agents, neuromuscular blocking drugs, anti-epileptic drugs, adrenocorticosteroids, insulin, proteins or enzymes involved in regulation of insulin, incretins (GIP and GLP-1) or drugs mimicking incretin action such as Exenatide and sitagliptin, thyroid hormones, growth hormone, ACTH, oestrogen, testosterone, anti diuretic hormone, diuretics, all kinds of blood products such as heparin and EPO, beta-blocking agents, cytotoxic agents, anti-viral drugs, anti-bacterial agents, anti fungal agents, anti-parasitic drugs, anti-coagulation drugs, anti-inflammatory drugs, anti-asthma drugs, and anti-COPD drugs.

55. A fusion protein according to claim 51, wherein the first molecule is selected from the list of sildenafil citrate, opiates, morphine, vitamins, hormones involved in pregnancy such as LH and FSH, hormones involved in sex changes, anti-contraceptives, and antibodies.

56. A fusion protein according to claim 1, wherein the first molecule is selected from the list of single-chain Fv, dAb or domain antibody, nanobody, VHH, diabody, V-NAR, ScFab, CTL-4, tendamistat, 10th fibronectin type 3 domain, neocarzinostatin, CBM4-2, Lipocalins, T-cell receptor, Protein A domain (protein Z), Im9, Designed A R proteins, Zinc finger, pVIII, Avian pancreatic polypeptide, GCN4, WW domain, Src homology domain 3 (SH3), Src homology domain 2, PDZ domains, TEM-1, D-lactamase, GFP, Thioredoxin, Staphylococcal nuclease, PHD-finger, CL-2, BPTI, APPI, HPSTI, Ecotin, LACI-D1, LDTI, MTI-II, Scorpion toxins, Insect defensin A peptide, EETI-II, Min-23, CBD, PBP, cytochrome b562, LdI receptor domain A, D-chrystallin, ubiquitin, transferrin and C-type lectin-like domain.

57. A pharmaceutical composition comprising the fusion protein according to claim 1 and one or more pharmaceutically acceptable excipients, diluents or carriers.

58. The fusion protein according to claim 1 for use as a medicament.

59. The fusion protein according to claim 1 for use in the treatment of cancer, psychosis, depression, Parkinsons disease, seizure, neuromuscular diseases, epilepsia, diabetes, bacterial or viral infections, fungus infections, coagulation disorders, asthma or COPD.

60. The fusion protein according to claim 1 for use in the treatment of an inflammatory condition, autoimmune disorder or a disorder involving undesired angiogenesis.

61. Use of the a fusion protein according to claim 1 in the preparation of a medicament for the treatment of a disease or disorder.

62. A method of treating a disease or disorder, wherein said method comprises administering to a subject in need of such treatment a therapeutically effective amount of the fusion protein according to claim 1.

63. A nucleic acid construct, encoding the fusion protein according to claim 1.

64. A method for preparing the fusion protein according to claim 1, wherein the first molecule is a polypeptide such as a cytokine, said method comprising the following steps: i) providing an expression system comprising a nucleotide encoding the polypeptide, ii) providing an expression system comprising a nucleotide encoding the monovalent immunoglobulin or a fragment thereof, iii) expressing said polypeptide, and said monovalent immunoglobulin or fragment thereof iv) recovering and purifying said expressed peptides, and v) combining the purified proteins by covalent binding.

65. A method of preparing the fusion protein according to claim 1, wherein the first molecule is a polypeptide, such as a cytokine, said method comprising the following steps: i) providing a nucleic acid construct with a nucleotide sequence encoding the polypeptide and a nucleotide sequence encoding the monovalent immunoglobulin or fragment thereof, wherein said nucleotide sequence encoding the polypeptide and the nucleotide sequence encoding the monovalent immunoglobulin or fragment thereof are operably linked together, ii) providing a cell expression system for producing said fusion protein, iii) producing said fusion protein by expressing said nucleic acid construct in cells of the cell expression system of ii), and iv) recovering said expressed fusion protein from the culture medium or cells.

66. A host cell comprising a nucleic acid construct according to claim 63, wherein said host cell is a prokaryotic cell, such as an E. coli cell or a eukaryotic cell.


SEQUENCE LISTING (56 sequences)

SEQ ID NO: 1 - DNA, 321 bp, Homo sapiens
cgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 2 - protein, 107 aa, Homo sapiens [sequence omitted]

SEQ ID NO: 3 - DNA, 327 bp, Homo sapiens
accgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacagaagctacagctgccaggtcacgcatgaagggagcaccgtggagaagacagtggcccctacagaatgttca

SEQ ID NO: 4 - protein, 109 aa, Homo sapiens
TVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 5 - DNA, 366 bp, Homo sapiens
gaagtgcagctggtggagtctgggggaggcttggtacagcctgacaggtccctgagactctcctgtgcagcctctggattcacctttcatgattatgccatgcactgggtccggcaagctccagggaagggcctggagtgggtctcaactattagttggaatagtggtaccataggctatgcggactctgtgaagggccgattcaccatctccagagacaacgccaagaactccctgtatctgcaaatgaacagtctgagagctgaggacacggccttgtattactgtgcaaaagatatacagtacggcaactactactacggtatggacgtctggggccaagggaccacggtcaccgtctcctca

SEQ ID NO: 6 - protein, 122 aa, Homo sapiens
EVQLVESGGGLVQPDRSLRLSCAASGFTFHDYAMHWVRQAPGKGLEWVSTISWNSGTIGYADSVKGRFTISRDNAKNSLYLQMNSLRAEDTALYYCAKDIQYGNYYYGMDVWGQGTTVTVSS

SEQ ID NO: 7 - DNA, 354 bp, Mus musculus
gaggttcagctgcagcagtctggggcagagcttgtgaaaccaggggcctcagtcaagttgtcctgcacagcttctggcttcaacattaaagacacctatatccactgggtgaagcagaggcctgaacagggcctggagtgggttggaaggattgatcctgcgactggcaatactagatatgacccgaagttccagggcaaggccactataacagctgacacatcctccaacacagcctacctgcaactcagcagcctgacatctgaggacactgccgtctattactgtgctagttttaggccggggtatgctctggactactggggtcaaggaacctcagtcaccgtctcctca

SEQ ID NO: 8 - protein, 118 aa, Mus musculus
EVQLQQSGAELVKPGASVKLSCTASGFNIKDTYIHWVKQRPEQGLEWVGRIDPATGNTRYDPKFQGKATITADTSSNTAYLQLSSLTSEDTAVYYCASFRPGYALDYWGQGTSVTVSS

SEQ ID NO: 9 - DNA, 321 bp, Homo sapiens
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggccgatcaccttcggccaagggacacgactggagattaaa

SEQ ID NO: 10 - protein, 107 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIK

SEQ ID NO: 11 - DNA, 321 bp, Mus musculus
gacattgtgatgacccagtctcacaaattcatgtccacatcagttggagacagggtcagcttcacctgcaaggccagtcaggatgtgtttactgctgtagcctggtatcaacaaaaaccagggcaatctcctaaactactgatttactgggcatccacccggcgcactggagtccctgatcgcttcacaggcagtggatctgggacagattatactctcaccatcagcagtgtgcaggctgaagacctggcactttattactgtcagcaacattttagcactcctccgacgttcggtggaggcaccaagctggaaatcaaa

SEQ ID NO: 12 - protein, 107 aa, Mus musculus
DIVMTQSHKFMSTSVGDRVSFTCKASQDVFTAVAWYQQKPGQSPKLLIYWASTRRTGVPDRFTGSGSGTDYTLTISSVQAEDLALYYCQQHFSTPPTFGGGTKLEIK

SEQ ID NO: 13 - DNA, 1588 bp, Homo sapiens
gctagcaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttggtgagaggccagcacagggagggagggtgtctgctggaagccaggctcagccctcctgcctggacgcaccccggctgtgcagccccagcccagggcagcaaggcatgccccatctgtctcctcacccggaggcctctgaccaccccactcatgctcagggagagggtcttctggatttttccaccaggctccgggcagccacaggctggatgcccctaccccaggccctgcgcatacaggggcaggtgctgcgctcagacctgccaagagccatatccgggaggaccctgcccctgacctaagcccaccccaaaggccaaactctccactccctcagctcagacaccttctctcctcccagatctgagtaactcccaatcttctctctgcagagtccaaatatggtcccccatgcccatcatgcccaggtaagccaacccaggcctcgccctccagctcaaggcgggacaggtgccctagagtagcctgcatccagggacaggccccagccgggtgctgacgcatccacctccatctcttcctcagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaaggtgggacccacggggtgcgagggccacatggacagaggtcagctcggcccaccctctgccctgggagtgaccgctgtgccaacctctgtccctacagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 14 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 15 - DNA, 1588 bp, Homo sapiens
gctagcaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttggtgagaggccagcacagggagggagggtgtctgctggaagccaggctcagccctcctgcctggacgcaccccggctgtgcagccccagcccagggcagcaaggcatgccccatctgtctcctcacccggaggcctctgaccaccccactcatgctcagggagagggtcttctggatttttccaccaggctccgggcagccacaggctggatgcccctaccccaggccctgcgcatacaggggcaggtgctgcgctcagacctgccaagagccatatccgggaggaccctgcccctgacctaagcccaccccaaaggccaaactctccactccctcagctcagacaccttctctcctcccagatctgagtaactcccaatcttctctctgcagagtccaaatatggtcccccatgcccaccatgcccgggtaagccaacccaggcctcgccctccagctcaaggcgggacaggtgccctagagtagcctgcatccagggacaggccccagccgggtgctgacgcatccacctccatctcttcctcagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaaggtgggacccacggggtgcgagggccacatggacagaggtcagctcggcccaccctctgccctgggagtgaccgctgtgccaacctctgtccctacagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 16 - protein, 315 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 17 - protein, 105 aa, Homo sapiens
QPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 18 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 21 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 22 - DNA, 36 bp, artificial
  primer
agccaccgtacgtttgatttccagcttggtgcctcc

SEQ ID NO: 23 - DNA, 44 bp, artificial
  primer
gatgcaagcttgccgccaccatggagtcacagattcaggcattt

SEQ ID NO: 24 - DNA, 42 bp, artificial
  primer
cgatgggcccttggtgctggctgaggagacggtgactgaggt

SEQ ID NO: 25 - DNA, 44 bp, artificial
  primer
gatgcaagcttgccgccaccatgaaatgcagctgggttatcttc

SEQ ID NO: 26 - DNA, 23 bp, artificial
  primer
tgtactttggcctctctgggata

SEQ ID NO: 27 - DNA, 29 bp, artificial
  primer
ctggagattaaacgtacggtggctgcacc

SEQ ID NO: 28 - DNA, 45 bp, artificial
  primer
gcgactaagcttgccgccaccatggaagccccagctcagcttctc

SEQ ID NO: 29 - DNA, 44 bp, artificial
  primer
gctgaaagcttgccgccaccatggagttgggactgagctggatt

SEQ ID NO: 30 - DNA, 23 bp, artificial
  primer
gtagtctgagcagtactcgttgc

SEQ ID NO: 31 - DNA, 23 bp, artificial
  primer
gaagacttaaggcagcggcagaa

SEQ ID NO: 32 - DNA, 23 bp, artificial
  primer
ggtcagggcgcctgagttccacg

SEQ ID NO: 33 - DNA, 23 bp, artificial
  primer
atgcaggctactctagggcacct

SEQ ID NO: 34 - DNA, 36 bp, artificial
  primer
gaagaccgatgggcccttggtgctagctgaggagac

SEQ ID NO: 35 - DNA, 32 bp, artificial
  primer
tgagaattcggtgggtgctttatttccatgct

SEQ ID NO: 36 - DNA, 33 bp, artificial
  primer
gtagaagcttaccatcgcggatagacaagaacc

SEQ ID NO: 37 - DNA, 33 bp, artificial
  primer
ggtcccccatgcccaccatgcccgggtaagcca

SEQ ID NO: 38 - DNA, 33 bp, artificial
  primer
tggcttacccgggcatggtgggcatgggggacc

SEQ ID NO: 39 - DNA, 26 bp, artificial
  primer
tgttaactgctcactggatggtggga

SEQ ID NO: 40 - DNA, 27 bp, artificial
  primer
tccctgggcacaattttcttgtccacc

SEQ ID NO: 41 - DNA, 31 bp, artificial
  primer
tgaaagcttctaatacgactcactatagggc

SEQ ID NO: 42 - DNA, 54 bp, artificial
  primer
tgaaagcttctaatacgactcactatagggcaagcagtggtatcaacgcagagt

SEQ ID NO: 43 - DNA, 62 bp, artificial
  primer
tcttcttcctgatggcagtggttacaggggtcaattcagaggtccagctgcagcagactgga

SEQ ID NO: 44 - DNA, 61 bp, artificial
  primer
gataagcttgccgccaccatgaaatgcagctgggttatcttcttcctgatggcagtggtta

SEQ ID NO: 45 - DNA, 45 bp, artificial
  primer
ggatgggcccttggtgctggccgcagagacagtgaccagagtccc

SEQ ID NO: 46 - DNA, 64 bp, artificial
  primer
cctcatgtccctgctgttctgggtatctggtacctgtggggacgttgtgatgacccagactcca

SEQ ID NO: 47 - DNA, 62 bp, artificial
  primer
acgaagcttgccgccaccatggaatcacagactcaggtcctcatgtccctgctgttctgggt

SEQ ID NO: 48 - DNA, 44 bp, artificial
  primer
aactcccaatcttctctctgcagctcaaggcgggacaggtgccc

SEQ ID NO: 49 - DNA, 44 bp, artificial
  primer
gggcacctgtcccgccttgagctgcagagagaagattgggagtt

SEQ ID NO: 50 - DNA, 22 bp, artificial
  primer
gggagtagagtcctgaggactg

SEQ ID NO: 51 - DNA, 22 bp, artificial
  primer
tatccaccttccactgtacttt

SEQ ID NO: 52 - DNA, 45 bp, artificial
  primer
cgatggaagcttgccgccaccatggaattggggctgagctgggtt

SEQ ID NO: 53 - DNA, 36 bp, artificial; identical to SEQ ID NO: 34
  primer

SEQ ID NO: 54 - protein, 19 aa, artificial
  synthetic peptide
VAPEFLGGPSVFLFPPKPK

SEQ ID NO: 55 - protein, 32 aa, homo sapiens
KPSNTKVDKRVESKYGPPCPSCPAPEFLGGPS

SEQ ID NO: 56 - protein, 20 aa, artificial
  synthetic peptide
KPSNTKVDKRVAPEFLGGPS